(19) United States
(12) Patent Application Publication
(10) Pub. No.: US 2003/0235860 A1
(43) Pub. Date: Dec. 25, 2003

(54) Interactions between AR, ER, TR2, and TR4

(76) Inventor: Chawnshang Chang, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 10/366,811

(22) Filed: Feb. 13, 2003

Related U.S. Application Data
(63) Continuation-in-part of application No. 09/711,585, filed on Nov. 13, 2000.
(60) Provisional application No. 60/165,300, filed on Nov. 12, 1999.

(51) Int. Cl. G01N 33/53; C07K 14/715; A61K 38/17

(52) U.S. Cl. 435/7.1; 514/12

(57) ABSTRACT

Disclosed are compositions and methods related to TR2, TR4, androgen receptor, and estrogen receptor and the interactions between these proteins.

This application claims priority to provisional application Serial No. 60/165,300 filed on Nov. 12, 1999 and U.S. patent application Ser. No. 09/711,585 filed on Nov. 13, 2000, which applications are herein incorporated by reference in their entirety. This work is supported by NIH grants, DK47258, DK56784, and DK51346.

### I. BACKGROUND

Testicular orphan nuclear receptor 2 (TR2) and testicular orphan nuclear receptor 4 (TR4) are members of the steroid receptor superfamily of nuclear receptors. Androgen receptor (AR) and estrogen receptor (ER) are also steroid receptors. Steroid receptors are transcription regulators, and they regulate transcription through binding DNA and interacting with other molecules involved in transcription. Steroid receptors typically function by binding a ligand which causes a conformational change. This conformational change promotes DNA binding. Along with this conformational change, steroid receptors interact to form dimers, both homo dimers and heterodimers. The formation of the dimers also typically promotes DNA binding, and thus transcription regulation. Different dimer pairs can bind different DNA binding sites and interact with different subsets of transcription factors. Thus, different dimer pairs cause different effects on transcription.

Disclosed herein, are the following heterodimer pairs, TR2-ER, TR4-ER, TR2-AR, and TR4-AR and these heterodimer pairs cause down regulation at of transcription activity at each cognate transcription site.

### II. SUMMARY

In accordance with the purposes of this invention, as embodied and broadly described herein, the disclosed compositions and methods, in one aspect, relates to TR2, TR4, AR, and ER and interactions between these molecules.

It is to be understood that both the foregoing general description and the following detailed description are exemplary and explanatory only and are not restrictive.

### III. BRIEF DESCRIPTION OF THE DRAWINGS

The accompanying drawings, which are incorporated in and constitute a part of this specification, illustrate several embodiments.

FIG. 1 shows a graphical illustration of the construction of AR deletion mutants.

FIG. 2 shows a graphical presentation of data from an experiment described below. In FIGS. 2, 2(A) illustrates data from an experiment analyzing TR4 interaction with AR in the mammalian two-hybrid system while FIG. 2(B) illustrates and experiment analyzing TR4 interaction with AR in modified mammalian one-hybrid system.

FIG. 3 shows a graphical representation of data from an experiment described below, an experiment studying AR repression of TR4-target gene expression. In FIG. 3(A) data is illustrated related to AR repression of TR4-mediated DR4-CAT and CNTFR-15-LUC transcriptional activity, while in FIG. 3(B) the data is related to AR repression of TR4-mediated HBV gene expression.

FIG. 4 shows a graphical representation of data from an experiment related to TR4 repression of AR-mediated transcriptional activity.

FIG. 5 illustrates data related to the specificity of negative regulation on AR-mediated MMTV-Luciferase activity by TR4.

FIG. 6 presents data related to the interaction between TR2 and ER in mammalian two-hybrid system. Values are presented as the mean±SD of three independent experiments.

FIG. 7 graphically illustrates data from an experiment related to the fact that ER inhibits TR2 transcriptional activity in H1299. Values presented are the mean±SD of three independent experiments.

FIG. 8 presents data from an experiment testing the effects of TR2 on estrogen-induced ER activity in different cell lines. All values represent the mean of duplicate samples, and similar results were obtained in three independent experiments.

FIG. 9 shows data from an experiment testing the inhibitory effect of TR2 on the induction of PR mRNA (A) and protein (B) by E2 in T47D cells.

FIG. 10 shows a graphical presentation of data from an experiment studying the inhibitory effect of a chimera receptor, TR2-ARp-TR2, on ER transcriptional activity. Values are the mean±SD of three independent determinations.

FIG. 11 shows the effects of TR2 on ERa-, ERb-, AR-, PR-, and GR-mediated transactivation. (A) TR2 repression of ER-mediated ERE-tk-CAT reporter genes in PC-3, H1299, MCF7, and T47D cells. Cells in 60-mm dishes were co-transfected with 2 mg of ERE-CAT, pSG5-ER, and/or pCMV-TR2 by calcium phosphate precipitation methods. 1 mg of b-galactosidase expression plasmid, pCMV-b-gal, was used as an internal control for transfection efficiency. CAT activity is presented relative to the response to ethanol, which is set as one. (B) MCF7-pBIG and MCF7-TR2 cells were stably transfected with doxycycline-inducible pBIG2i and pBIG2I-FLAG-TR2 plasmids, respectively. Cells were transfected with 2 mg of ERE-CAT and 1 mg of pCMV-b-gal and, after 16 hr, were treated with or without 2 mg/ml doxycycline. CAT activity is presented relative to the response to ethanol. FLAG-TR2 induction was monitored by Western blotting using anti-FLAG antibody (M2), shown in the upper panel. (C) The effect of TR2 on endogenous ER target gene PR expression at protein and mRNA levels. T-47D cells seeded in 60-mm dishes were transfected with 10 mg of pCMV or pCMV-TR2 for 16 h, followed by treatment with 10 nM E2 for another 16 h. Cell extracts (50 mg) and mRNA (15 mg) were used for Western blotting with anti-PR antibody (H-190) and Northern Blotting, respectively. Relative mRNA expression amounts were normalized by 28S expression and quantitated by ImageQuant V.1.2 (Molecular Dynamics). (D) TR2 effect on the transactivation of other steroid receptors. Methods used are the same as described in (A). ERE-Luc and MMTV-Luc reporter genes were used for examination of ERb and AR transactivation, respectively. MMTV-CAT reporter was used for PR and GR transactivation. Luciferase activity was analyzed following manufacturer's instructions (Promega). 10−8 M of ligands, E2, DHT, progesterone (P), and dexamethasone (Dex), were used as indicated. Values are the means±SD of three independent experiments

FIG. 12 shows the physical association analysis between TR2 and ER. (A) 500 mg of total proteins from MCF7 cells treated with ethanol, 10 nM E2, or 1 mM tamoxifen for 24 h were immunoprecipitated with normal rabbit IgG or rabbit anti-ER antibody (H-184) as indicated. The immunoprecipitates were subjected to Western blotting with anti-ER (1:1000, H-184) or anti-TR2 (1:1000, G204) antibodies. (B) In vitro interaction analysis. The GST and GST-TR2 fusion proteins were purified as instructed by the manufacturer (Amersham Pharmacia). 5 ml of in vitro-translated [35S]-labeled AR, ER, and RXR were incubated with the GST or GST-TR2 bound to glutathione-Sepharose beads in a pull-down assay. After extensive washing, bead-bound protein complexes were loaded onto 8% SDS-PAGE and analyzed by Phosphorlmager The input represents 20% amount of [35S]-labeled proteins used in each pull-down assay. (C) Ligand effects on the interaction between ER and TR2. Three kinds of treatments (ethanol, 10 nM E2, 1 mM Tamoxifen) were added, individually, in each GST pull-down reaction as indicated The input represents 10% amount of [35S]-labeled ER used in each pull-down assay.

FIG. 13 shows the mapping the interaction domains on ER and TR2. (A) The construction of GST-ER fragments is illustrated schematically on the upper panel. GST alone and GST fusion proteins were purified as described by the manufacturer instructions. 5 ml of [35S]-TR2 was incubated with GST or GST-ER fusion proteins bound to glutathione-Sepharose beads in the absence or presence of 1 mM E2. After extensive washing, bead-bound protein complexes were loaded onto 8% SDS-PAGE and analyzed by PhosphorImager. (B) Schematic representation of GST-TR2 constructs is illustrated on the upper panel. GST or three GST-TR2 fusion proteins were purified and incubated with 5 ml of [35S]-ER in the pull-down assay. The input represents 10% amount of [35S]-labeled proteins used in each pull-down assay. DBD, DNA binding domain; LBD, ligand binding domain.

FIG. 14 shows ER-#6 serves as ER-TR2 interaction blocker capable of reversing the suppression of ER by TR2. (A) ER-#6 blocks ER-TR2 interaction in GST pull-down assay. GST and GST-TR2 fusion proteins were purified as described by the manufacturer (Amersham Pharmacia). Glutathione-Sepharose beads bound GST-TR2 were then incubated with 5 ml of [35S]-ER with increasing amounts of HA-ER-#6, which was in vitro translated from pcDNA3-HA-ER-#6 plasmids, for 2 h at 4° C. in the absence of E2. After extensive washing, bead-bound protein complexes were loaded onto 8% SDS-PAGE and analyzed by PhosphorImager. The input represents 10% amount of [35S]-ER used in each pull-down assay. (B) ER-#6 inhibits ER-TR2 interaction in mammalian two-hybrid system. PC-3 cells plated on 60-mm dishes were co-transfected with 2 mg of pG5-CAT reporter with plasmids as indicated. 1 mg of pCMV-b-gal was also used as an internal control for transfection efficiency. CAT activity was analyzed in the presence of 10-8 M E2. (C) ER-#6 reverses TR2-mediated suppression of ER transactivation. MCF7-TR2 cells were co-transfected with 2 mg of ERE-CAT, 1 mg of pCMV-b-gal, and 7 mg of pcDNA3 or pcDNA3-HA-ER-#6. After 16 h transfection, cells were treated with ethanol, 10 nM E2, and/or increasing amount of doxycycline, as indicated, for another 16 h. CAT activity is presented relative to the response to ethanol, set as one. Values are the means±SD of three independent experiments.

FIG. 15 shows the enhancement of ER transcriptional activity by administration of antisense TR2 in MCF7 cells. (A) MCF7 cells cultured in 60-mm dishes were transfected with 2.5 mg of pcDNA3-TR2-1f AS, pIRES-TR2-N AS, and/or pCMV-TR2, as indicated. SuperFect transfection kit (Qiagen) was used for transfections. The total amount of plasmids in each dish was made up to 5 mg by adding pcDNA3 parent vector. After 16 h transfection, cell extracts were obtained and subjected to Western blotting with anti-TR2 (G204) and anti-b-actin antibodies. β-actin expression levels serve as loading control. (B) 0.125 mg of ERE-Luc with increasing amounts of pcDNA3-TR2-lf AS, pIRES-TR2-N AS, or pcDNA3-HA-ER-#6 were transfected into MCF7 cells, using SuperFect transfection kit (Qiagen). Luciferase activity was analyzed according to manufacturer's instructions (Promega). Luciferase activity is presented relative to response to ethanol, set as' 1. Values are the means±SD of three independent experiments.

FIG. 16 shows the interference with ER binding to ERE by ER-TR2 heterodimer formation. (A) Interruption of ER binding to ERE by TR2 in EMSA. [32P]-end-labeled ERE probe (4×108 dpm/mg) was incubated with in vitro translated TR2 and ER proteins (ratios from 1:1 to 1:4) in EMSA binding buffer and analyzed on a 5% acrylamide native gel containing 2.5% glycerol. 1 ml of anti-ERa monoclonal antibody (C-314) was added for antibody supershifts (lane 5 and 6). A 100-fold excess molar of unlabeled ERE probe was added as a cold competitor (lane 7 and 8). Ethanol or 10 mM E2 was added as indicated. The migration positions of the supershifted band formed by Ab-ER-ERE are indicated as an arrowhead, ns, non-specific binding. (B) ER homodimeric formation is disrupted by TR2, but rescued by ER-#6. GST-ER-#3 (LBD) and GST proteins were purified as described by the manufacturer (Amersham Pharmacia). In vitro translated [35S]-ER with increasing amounts of [35S]-TR2 were co-incubated with GST-ER-#3 or GST alone which were bound to glutathione-Sepharose beads. ER-#6 peptide was obtained using the thrombin protease cleavage method (ROCHE) to release ER-#6 peptide from bead-bound GST-ER-#6. Equal amounts of GST-ER-#3 and GST-ER-#6 were used as determined by a coomassie-staining gel. After extensive washing, bead-bound protein complexes were loaded onto 8% SDS-PAGE and analyzed by PhosphorImager (Molecular Dynamics). The input represents 0.5 ml of [35S]-labeled ER and TR2 as used in each reaction.

FIG. 17 shows the TR2 suppresses E2-induced breast cancer cell growth and G1/S transition. (A) Growth assays were performed by MTT method as instructed by manufacturer (Sigma). 5×103 MCF7-pBIG and MCF7-TR2 cells were seeded in 24-well plates and incubated in RPMI with 10% CD-FCS for 48 h. Cells were treated with ethanol, 10 nM E2, and/or 2 mg/ml doxycycline as indicated. After 3- and 5-day treatment, cells were harvested for MTT assay. Data represents the relative folds of MTT O.D.570 values at day 5 divided by that at day 3. (B) The inhibition of E2-induced G1/S transition by TR2 in MCF7-TR2 cells. Cells were incubated in RPMI with 10% CD-FCS for 48 h and then treated with ethanol, 10 nM E2 and 2 mg/ml doxycycline, as indicated, for 72 h. Cells were then trypsinized and fixed overnight in 70% Ethanol. After cells were incubated with 1 mg/ml RNase A (Sigma) and propidium iodide (Roche Molecular Biochemicals), the DNA contents of cells were measured by a flow cytometry. (C) Percentage of growth suppression by TR2. MCF7-TR2 cells were seeded at 5×103 cells per well in 24-well plates. Following cultured in RPMI medium with 10% CD-FCS for 48 h, cells were treated with 10 nM E2, 5 mM Tamoxifen, or 1 mM all-trans RA in the presence or absence of 2 mg/ml doxycycline for up to 8 days. The treatments were changed every two days. The indices of cell growth were determined by MTT assay. The MTT values from cells without doxycycline were set as 100% in each kind of treatment. Values are represented by the percentage of growth suppression from cells with doxycycline to that without doxycycline.

FIG. 18 shows the selective suppression of ER transactivation by TR4. (A), HI 299 cells were either co-transfected with ERE-CAT reporter plasmid and the expression plasmid for wild-type ERα or ERβ, or co-transfected with MMTV-LUC and PR plasmid, with increasing amounts of the TR4 expression vector in the absence or presence of 10 nM E2 (for ERE-CAT) or 10 nM progesterone (for MMTV-LUC). (B), MCF-7 cells were co-transfected with ERE-CAT with increasing amounts of the TR4 expression vector. Cells were then treated with or without 10 nM E2 for ERE-CAT. The CMV-β gal (CAT assay) or SV-40 Renilla luciferase (Luciferase assay) internal control plasmid was co-transfected to correct for transfection efficiency. The CAT activity fold was determined relative to activity in the absence of TR4 and E2. And the Luciferase activity fold was calculated relative to activity in the absence of TR4 and progesterone. Bars represent the means±S.D. of three individual experiments.

FIG. 19 shows the interaction between TR4 and ER. (A), In vitro interaction of TR4 with ER, [³⁵S] labeled AR, ER, and RXRct were incubated with GST-TR4 or GST bound glutathione-sepharose beads in a pull-down assay. (B), [³⁵S] labeled TR4 was incubated with GST-ER-LBD or GST bound glutathione-sepharose beads in the absence or presence of 1 μM E2 The input represents 20% of the amount of labeled protein used in the pull-down assay.

FIG. 20 shows that TR4-LBD is essential for the suppression effect of TR4 on ER. (A), Constructs used in GST-pull down assay and transient transfection. (B), GST pull-down assay used [S³⁵] labeled ER and purified GST-fusion protein or GST bound glutathione-Sepharose beads. The input represents 20% of the amount of labeled protein used in the pull-down assay. (C), H1299 cells were co-transfected with ERE-CAT reporter plasmid and the expression plasmid for ERα and increasing amounts of full-length TR4 or C-terminal deletion TR4 expression plasmids. Transfected cells were treated with 10 nM E2. The E2 induced CAT activity of lysates from cells transfected with ERα expression plasmid only was used as 100% control. Bars represent the means±S.D. of three individual experiments.

FIG. 21(A), shows [P³²]labeled ERE was either incubated with in vitro expressed ER with increasing amount of in vitro expressed TR4 or TR4 alone as indicated at the top of each lane and resolved in 5% native gel. ER-ERE was further supershifted by ER antibody (C-314). The arrow indicates ER-ERE complex and the arrowhead indicates supershift complex. FIG. 21(B), shows [S³⁵] labeled ER was incubated with purified GST-ER-LBD or GST bound glutathione-Sepharose beads with increasing amounts of TR4 in the absence or presence of 1 μM E2. The input represents 20% of the amount of labeled protein used in the pull-down assay.

FIG. 22 shows inhibition of ER endogenous target gene pS2 by Dox-induced TR4 expression. (A), TR4 expression was induced by Dox in stably transfected MCF-7 cells (MCF-7-TR4). RNA from MCF-7-TR4 cells was isolated in the absence or presence of 2 μg/ml Dox. hTR4 N-terminal was used as probe to perform Northern blotting. (B), MCF-7-TR4 cells and MCF-7-pBIG were transfected with ERE-CAT reporter plasmid. The cells were then treated with 10 nM E2 for ERE-CAT after 2 μg/ml Dox treatment for 24 h. (C), RNA was isolated from MCF-7-TR4 and MCF-7-pBIG cells with or without 2 μg/ml Dox treatment for 24 hr and then treated with 10 nM E2 or ethanol for 2 days. Northern blotting was performed to determine the expression level of pS2 gene. The β-actin labeling was used to demonstrate the equal loading of RNA amount.

FIG. 23 shows suppression of E2-induced MCF-7 cells growth and cyclin D1 expression by Dox-induced TR4 expression. (A), MCF-7-TR4 and MCF-7-pBIG cells were estrogen deprived for 4 days, then treated with 2 Ξg/ml Dox treatment for 24 h, and then treated with 10 nM E2 to induce cell proliferation, and then the cells were counted at different times. The cell number after 24 h was used as 100% control. Bars represent the means±S.D. of three individual experiments. (B), The cell lysates from MCF-7-TR4 and MCF-7-pBIG cells treated in the presence or absence of 2 μg/ml Dox for 24 hr were collected after 12 h with or without the treatment with 10 nM E2. Cyclin D1 protein were probed by cyclin D1 rabbit polyclonal Ab (H295) and detected by alkaline phosphatase conjugated secondary Ab.

FIG. 24 shows that the TR2 suppresses AR-mediated transactivation. 2 μg AR transactivation reporter gene MMTV-CAT (A) and PSA-CAT (B), 0.5 μg AR and GR expression plasmid pCMV-AR (Lane 2-5), pSG5-GR (A, Lane 7-10) were transfected into PC-3 cells. Increasing amounts (2, 4, and 7 %μg) of TR2 expression plasmid pCMV-TR2 (Lane 3-5,8-10) were also transfected into PC-3 cells. After 18 h transfection, the cells were treated with either 10 nM DHT (A and B, Lane 1-5), or 10 nM dexamethasone (A, Lane 6-10). CAT assay was performed as described in materials and methods.

FIG. 25 shows that the suppression of AR-mediated transactivation by TR2 is not due to competing for the limited coregulator availability by these two receptors. 2 μg AR transactivation reporter gene MMTV-CAT, 0.5 μg AR expression plasmid pCMV-AR, the increasing amounts of (2, 4, and 7 μg) TR2 expression plasmid pCMV-TR2 were transfected into PC-3 cells. 1 μg each of pSG5-SRC-1 (Lane 6-10), pSG5-CBP (Lane 11-15), pSG5-ARA70 (Lane 16-20), pSG5-TIF II (Lane 21-25), pSG5-ARA54 (Lane 26-30), and pSG5-ARA55 (Lane 31-35) were also transfected. CAT assay was performed as described in materials and methods.

FIG. 26 shows that TR2 suppresses AR target gene PSA expression. Vector control (Lane 1 and 2), and TR2 expression plasmid pCMV-TR2 (Lane 3 and 4) were transfected into prostate cancer LNCaP cells without (Lane 1 and 3) or with (Lane 2 and 4) treatment of 10 nM DHT, and Northern blot was performed as described in materials and method.

FIG. 27 shows the interaction between AR and TR2 in a GST Pull-down assay. The GST-TR2 fusion protein and GST control were purified according to manufacturer's protocol. 5 μl in vitro-translated 35S methionine-labeled AR (Lane 4, 6, and 8) and RXRα (Lane 3, 5, and 7) were incubated with the GST-TR2 (Lane 4-7) and GST (Lane 3 and 8) bound glutathione-sepharose beads. The pull-down complex was loaded on 10% SDS-PAGE and visualized by autoradiography.

### IV. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Abbreviations: CAT, chloramphenicol acetyltransferase; DBD, DNA-binding domain; E2, 17β-estradiol; ER, estrogen receptor; ERE, estrogen response element; GST, glutathione S-transferase; LBD, ligand-binding domain; PR, progesterone receptor; TR2, TR2 orphan receptor TR2, TR2 orphan receptor; TR4, TR4 orphan receptor; RA, retinoic acid; PPARα, peroxisome proliferator-activated receptor α; CAT, chloramphenicol acetyltransferase; RAR, retinoic acid receptor; PPRE, peroxisome proliferator response element; 1,25-(OH)₂D₃, 1,25-dihydroxyvitamin D₃; Kd, equilibrium dissociation constant, TR4 associated protein; AR, androgen receptor; GR, glucocorticoid receptor; TR, thyroid hormone receptor; TR4RE, TR4 response element; TR4-N, TR4-N terminus; TR4-DL, TR4 DNA binding domain (DBD) and ligand binding domain (LBD); DR, direct repeat; HDACs, histone deacetylases; TSA, Trichostatin; CAT, EMSA, electrophoretic mobility shift assay; LUC, luciferase; -UL, minus uronolactone.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Transcription activity as used herein refers to the activity a particular protein has as an activator of transcription. There are many ways that this activity can be determined, for example, CAT assays or luceriferase assays are two examples used herein.

A system refers to a collection of components which have a certain function or activity. For example, a cell that is transfected with a particular nucleic acid that is expressed can be a system that can be used for the expression of the cognate nucleic acid.

Interacts means that two (or more) molecules touch one another in a way beyond the touching that takes place because of random contacts between molecules. “Interacts” can be thought of as “binding” between two or more molecules, and therefore can have dissociation and association constants as well as equilibrium constants.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TR4 or TR2 or AR or ER is disclosed and discussed and a number of modifications that can be made to a number of molecules including the TR4 or TR2 or AR or ER are discussed, specifically contemplated is each and every combination and permutation of TR4 or TR2 or AR or ER and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

B. Compositions and Methods

Disclosed are relationships between TR2 and AR, TR2 and ER, TR4, and AR, and TR4 and ER. These relationships include the ability to repress the other's transcriptional activation activity. TR2 functions as a repressor of AR and ER transactivation activity, TR4 functions as a transcriptional repressor of AR and ER transactivation activity, ER functions as a transcriptional repressor of TR2 and TR4 transactivation activity, and AR functions as a transcriptional repressor of TR2 and TR4 transactivation activity. It is shown that fragments of these proteins retain these activities. As it is well understood that AR, ER, TR2, and TR4 are associated with a wide variety of pathological conditions including a number of cancers such as prostate cancer, breast cancer, and cervical carcinomas, the inhibition of their activity can be used to inhibit aberrant cell growth. It is also understood that the interactions that exist between these four proteins and their fragments and variants define new regions of the molecules for drug targets. The disclosed compositions can be used in combination with any other treatment, method, or composition for affecting the transactivation activity.

For example, disclosed are compositions comprising a fragment of TR2, wherein the composition interacts with ER, such that ER transcriptional activity is decreased relative to transcriptional activity in the absence of the composition.

Disclosed are compositions comprising a fragment of TR2, wherein the composition interacts with ER, such that ER transcriptional activity is decreased relative to transcriptional activity in the absence of the composition, wherein the fragment of TR2 has at least 80%, 85%, 90%, or 95% identity to amino acids 88-196 of SEQ ID NO:10, as well as compositions, wherein any variation between the TR2 and the sequence set forth in SEQ ID NO: 10 is a considered a conserved variation.

Disclosed are compositions, wherein the composition reduces the transcription activity of ER, for example, wherein the composition reduces the transcription activity of ER by 10%, 25%, 50%, or 90%.

Also disclosed are compositions, wherein the composition reduces the G1/S transition of the cell cycle, for example, wherein the composition reduces the G1/S transition of the cell cycle by 10%, 25%, 50%, or 90%.

Disclosed are methods of inhibiting transcription activity of ER comprising administering any of the disclosed compositions related to ER, AR, TR2, or TR4, as well as molecules that interact with these.

Disclosed are methods of inhibiting TR2 transcription activity comprising administering a composition that binds TR2, wherein the composition is thereof, or a molecule that competitively competes with TR2 for ER binding.

Also disclosed are methods of identifying an inhibitor of an interaction between ER and TR2, comprising incubating a library of molecules with an ER, ER fragment, ER variant or combination, forming a mixture, and identifying the molecules that disrupt the interaction between the ER, ER fragment, ER variant, or combination and TR2, wherein the interaction disrupted comprises an interaction between the ER, ER fragment, ER variant, or combination and TR2 binding site.

Disclosed are methods, wherein the step of isolating comprises incubating the mixture with a molecule comprising TR2, TR2 fragment, TR2 variant, or combination.

Also disclosed are methods of identifying an inhibitor of an interaction between ER and TR2 comprising incubating a library of molecules with TR2, TR2 fragment, TR2 variant or combination forming a mixture, and identifying the molecules that disrupt the interaction between ER and the TR2, TR2 fragment, TR2 variant or combination, wherein the interaction disrupted comprises an interaction between the ER and the TR, TR2 fragment, TR2 variant or combination binding site.

Also disclosed are methods, wherein the step of isolating comprises incubating the mixture with molecule comprising ER, ER fragment, ER variant, or combination.

Disclosed are compositions comprising a fragment of ER, wherein the composition interacts with TR2, such that TR2 transcriptional activity is decreased relative to transcriptional activity in the absence of the composition, wherein the fragment comprises a polypeptide having at least 80%, 85%, 90%, or 95% identity to the sequence set forth in SEQ ID NO:32, the fragment comprises a polypeptide having at least 80%, 85%, 90%, or 95% identity to amino acids 312-340, set forth in SEQ ID NO:32, the fragment comprises a polypeptide having at least 80%, 85%, 90%, or 95% identity to amino acids 123-340, set forth in SEQ ID NO:32, or the fragment comprises a polypeptide having at least 80%, 85%, 90%, or 95% identity to amino acids 312-595, set forth in SEQ ID NO:32.

Also disclosed are compositions, wherein any variation between the ER and the sequence set forth in SEQ ID NO: 32 is a conserved variation or wherein the fragment comprises the sequence set forth in SEQ ID NO:32, or is a conserved variant thereof.

Disclosed are composition, wherein the compositions reduce the transcription activity of TR4, for example, wherein the composition reduces the transcription activity of TR4 by 10%, 25%, 50%, or 90%.

Disclosed are methods of inhibiting transcription activity of TR2 comprising administering any of the disclosed compositions, such as TR2, TR4 fragments, or TR4 variants.

Disclosed are methods of identifying inhibitors of ER transcription activity comprising mixing a compound with an ER, ER fragment, ER variant, or combination, and identifying compounds which compete with TR2 binding with the ER, ER fragment, or ER variant.

Disclosed are methods of identifying inhibitors of ER transcription activity comprising mixing a set of compounds with ER, ER fragment, ER variant, or combination and identifying compounds which compete with TR2 binding with ER, ER fragment, or ER variant.

A method for identifying compounds that mimic TR2 and ER interaction, comprising incubating a compound and ER, ER fragment, ER variant, or combination forming a compound-ER mixture, incubating TR2 with the compound-ER mixture, and determining if the TR2 inhibits compound-ER interaction.

Disclosed are methods for identifying compounds that mimic TR2 and ER interaction, comprising incubating a set of compounds and ER, ER fragment, ER variant, or combination forming a compound-ER mixture, incubating TR2 with the compound-ER mixture, and identifying the compounds which TR2 inhibits from forming a compound-ER interaction.

Disclosed are methods for identifying compounds that mimic TR2 and ER interaction, comprising incubating a compound and TR2, TR2 fragment, TR2 variant, or combination forming a compound-TR2 mixture, incubating ER with the compound-TR2 mixture, and determining if the ER inhibits compound-TR2 interaction.

Also disclosed are methods for identifying compounds that mimic TR2 and ER interaction, comprising incubating a set of compounds and TR2, TR2 fragment, TR2 variant, or combination forming a compound-TR2 mixture, incubating ER with the compound-TR2 mixture, and identifying the compounds which ER inhibits from forming a compound-TR2 interaction.

Disclosed are methods, wherein the TR comprises amino acids 88-196 of SEQ ID NO:10, for example, wherein the TR2 has a sequence of at least 80%, 85%, 90%, or 95% identity to amino acids 88-196 of SEQ ID NO:10, also for example, wherein any variation between the TR2 and the sequence set forth in SEQ ID NO: 10 is considered a conserved variation.

Disclosed are methods, wherein the identified compound binds ER with a kd less than or equal to 10⁻⁵ M, 10⁻⁶ M, 10⁻⁷ M, 10⁻⁸ M, 10⁻⁹ M, or 10⁻¹⁰ M, 10⁻¹¹ M, or 10⁻¹² M.

Disclosed are methods of claims, wherein the ER comprises a polypeptide having at least 80%, 85%, 90%, or 95% identity to the sequence set forth in SEQ ID NO:32, and/or wherein the fragment comprises a polypeptide having at least 80%, 85%, 90%, or 95% identity to amino acids 312-340, set forth in SEQ ID NO:32, the fragment comprises a polypeptide having at least 80%, 85%, 90%, or 95% identity to amino acids 123-340, set forth in SEQ ID NO:32, or the fragment comprises a polypeptide having at least 80%, 85%, 90%, or 95% identity to amino acids 312-595, set forth in SEQ ID NO:32, and for example, wherein any variation between the ER and the sequence set forth in SEQ ID NO: 32 is a conserved variation.

Disclosed are methods, wherein the ER, ER fragment, ER variant or combination comprises the sequence set forth in SEQ ID NO:32, the sequence set forth by amino acids 312-340 of the sequence set forth in SEQ ID NO:32, the sequence set forth by amino acids 123-340 of the sequence set forth in SEQ ID NO:32, the sequence set forth by amino acids 312-595 of the sequence set forth in SEQ ID NO:32, or is a conserved variant thereof, or is a fragment thereof.

Disclosed are methods, wherein the identified compound binds TR2 with a kd less than or equal to 10⁻⁵ M, 10⁻⁶ M, 10⁻⁷ M, 10⁻⁸ M, 10⁻⁹ M, or 10⁻¹⁰ M, 10⁻¹¹ M, or 10⁻¹² M.

Also disclosed are methods of inhibiting TR4 transcription activity comprising administering a composition that binds TR4, wherein the composition is AR or fragment thereof, or a molecule that competitively competes with TR4 for AR binding.

Disclosed are methods of inhibiting AR transcription activity comprising administering a composition that binds AR, wherein the composition is TR4 or fragment thereof, or a molecule that competitively competes with AR for TR4 binding.

Also disclosed are methods of identifying an inhibitor of AR transcription activity comprising mixing a compound with AR and identifying compounds which compete with TR4 interaction with AR.

Disclosed are methods of identifying inhibitors of AR transcription activity comprising mixing a set of compounds with AR, AR fragment, AR variant, or combination and identifying compounds which compete with the TR4 interaction with AR.

A method of identifying an inhibitor of an interaction between AR and TR4, comprising incubating a library of molecules with AR, AR fragment, AR variant, or combination forming a mixture, and identifying the molecules that disrupt the interaction between the AR, AR fragment, AR variant, or combination and TR4, wherein the interaction disrupted comprises an interaction between the AR, AR fragment, AR variant, or combination and TR4 binding site.

Disclosed are methods, wherein the step of isolating comprises incubating the mixture with molecule comprising TR4, TR4 fragment, TR4 variant or combination.

Disclosed are methods of identifying an inhibitor of an interaction between AR and TR4 comprising incubating a library of molecules with TR4, TR4 fragment, TR4 variant, or combination forming a mixture, and identifying the molecules that disrupt the interaction between AR and the TR4, TR4 fragment, TR4 variant, or combination, wherein the interaction disrupted comprises an interaction between the AR and the TR4, TR4 fragment, TR4 variant, or combination binding site.

Disclosed are methods, wherein the step of isolating comprises incubating the mixture with molecule comprising AR, AR fragment, AR variant or combination.

For example, disclosed are methods, wherein the AR, AR fragment, AR variant or combination comprises a polypeptide having at least 80%, 85%, 90%, or 95% identity to SEQ ID NO:31, as well as methods wherein any variation between the AR and the sequence set forth in SEQ ID NO: 31 is a conserved variation, or wherein the AR comprises the sequence set forth in SEQ ID NO:31, or is a conserved variant thereof, or is a fragment thereof.

Disclosed are methods, wherein the transcription activity of TR4 is reduced by 10%, 25%, 50%, or 90%.

Disclosed are methods, wherein the TR4, TR4 fragment, TR4 variant or combination comprises a polypeptide having at least 80%, 85%, 90%, or 95% identity to SEQ ID NO:16, as well as methods wherein any variation between the TR4 and the sequence set forth in SEQ ID NO:16 is a conserved variation, or wherein the TR4 comprises the sequence set forth in SEQ ID NO:16, or is a conserved variant thereof, or is a fragment thereof.

Disclosed are methods, wherein the transcription activity of AR is reduced by 10%, 25%, 50%, or 90%.

Disclosed are methods of inhibiting TR4 transcription activity comprising administering a composition that binds TR4, wherein the composition is ER or fragment thereof, or a molecule that competitively competes with TR4 for ER binding.

Also disclosed are methods of inhibiting ER transcription activity comprising administering a composition that binds ER, wherein the composition is TR4 or fragment thereof, or a molecule that competitively competes with ER for TR4 binding.

Disclosed are methods of identifying an inhibitor of ER transcription activity comprising mixing a compound with ER and identifying compounds which compete with TR4 interaction with ER.

Also disclosed are methods of identifying inhibitors of ER transcription activity comprising mixing a set of compounds with ER and identifying compounds which compete with TR4 interaction with ER.

Disclosed are methods of identifying an inhibitor of an interaction between ER and TR4, comprising incubating a library of molecules with ER, ER fragment, ER variant, or combination, forming a mixture, and identifying the molecules that disrupt the interaction between the ER, ER fragment, ER variant, or combination, and TR4, wherein the interaction disrupted comprises an interaction between the ER, ER fragment, ER variant, or combination, and TR4 binding site.

Disclosed are methods, wherein the step of isolating comprises incubating the mixture with molecule comprising TR4, TR4 fragment, TR4 variant or combination.

Also disclosed are methods of identifying an inhibitor of an interaction between ER and TR4 comprising incubating a library of molecules with TR4, TR4 fragment, TR4 variant, or combination, forming a mixture, and identifying the molecules that disrupt the interaction between ER and the TR4, TR4 fragment, TR4 variant, or combination, wherein the interaction disrupted comprises an interaction between the ER and the TR4, TR4 fragment, TR4 variant, or combination binding site.

Disclosed are methods, wherein the step of isolating comprises incubating the mixture with a molecule comprising an ER, ER fragment, ER variant or combination.

Disclosed are methods, wherein the ER, ER fragment, ER variant or combination comprises a polypeptide having at least 80%, 85%, 90%, or 95% identity to SEQ ID NO:32.

Also disclosed are methods, wherein any variation between the ER and the sequence set forth in SEQ ID NO: 32 is a conserved variation as well as methods wherein the ER comprises the sequence set forth in SEQ ID NO:32, or is a conserved variant thereof, or is a fragment thereof.

Disclosed are methods, wherein the transcription activity of TR4 is reduced by 10%, 25%, 50%, or 90%.

Disclosed are methods, wherein the TR4, TR4 fragment, TR4 variant or combination comprises a polypeptide having at least 80%, 85%, 90%, or 95% identity to SEQ ID NO: 16, as well as methods wherein any variation between the TR4 and the sequence set forth in SEQ ID NO: 16 is a conserved variation or for example, methods, wherein the TR4 comprises the sequence set forth in SEQ ID NO:16, or is a conserved variant thereof, or is a fragment thereof.

Disclosed are methods, wherein the transcription activity of ER is reduced by 10%, 25%, 50%, or 90%.

Disclosed are methods of inhibiting AR transcription activity comprising administering a composition that binds AR, wherein the composition is TR2 or fragment thereof, or a molecule that competitively competes with AR for TR2 binding.

Also disclosed are methods of identifying an inhibitor of AR transcription activity comprising mixing a compound with AR, AR fragment, AR variant, or combination and identifying compounds which compete with TR2 interaction with the AR, AR fragment, AR variant, or combination.

Disclosed are methods of identifying inhibitors of AR transcription activity comprising mixing a set of compounds with AR, AR fragment, AR variant, or combination and identifying compounds which compete with TR2 interaction with the AR, AR fragment, AR variant, or combination.

Disclosed are method of identifying an inhibitor of an interaction between AR and TR2, comprising incubating a library of molecules with AR, AR fragment, AR variant, or combination forming a mixture, and identifying the molecules that disrupt the interaction between the AR, AR fragment, AR variant, or combination and TR2, wherein the interaction disrupted comprises an interaction between the AR, AR fragment, AR variant, or combination and TR2 binding site.

Disclosed are methods, wherein the step of isolating comprises incubating the mixture with molecule comprising TR2, TR2 fragment, TR2 variant or combination.

Also disclosed are methods of identifying an inhibitor of an interaction between AR and TR2 comprising incubating a library of molecules with TR2, TR2 fragment, TR2 variant or combination forming a mixture, and identifying the molecules that disrupt the interaction between AR and the TR2, TR2 fragment, TR2 variant or combination, wherein the interaction disrupted comprises an interaction between the AR and the TR2, TR2 fragment, TR2 variant or combination binding site.

Disclosed are methods wherein the step of isolating comprises incubating the mixture with molecule comprising AR, AR fragment, AR variant or combination.

Also disclosed are methods, wherein the AR, AR fragment, AR variant or combination comprises a polypeptide having at least 80%, 85%, 90%, or 95% identity to SEQ ID NO:31, as well as methods wherein any variation between the AR and the sequence set forth in SEQ ID NO: 31 is a conserved variation, and methods wherein the AR comprises the sequence set forth in SEQ ID NO:31, or is a conserved variant thereof, or is a fragment thereof.

Disclosed are methods, wherein the transcription activity of TR2 is reduced by 10%, 25%, 50%, or 90%.

Disclosed are methods, wherein the TR2, TR2 fragment, TR2 variant or combination comprises a polypeptide having at least 80%, 85%, 90%, or 95% identity to SEQ ID NO:16, as well as methods wherein any variation between the TR2 and the sequence set forth in SEQ ID NO: 16 is a conserved variation, and for example, methods wherein the TR2 comprises the sequence set forth in SEQ ID NO: 16, or is a conserved variant thereof, or is a fragment thereof.

Disclosed are methods, wherein the transcription activity of AR is reduced by 10%, 25%, 50%, or 90%.

Disclosed are compositions that comprise combinations of TR2 and ER or TR2 and AR or TR4 and ER or TR4 and AR, as well as any other combination of TR2, TR4, ER, and AR.

Disclosed herein TR4 orphan receptor and the androgen receptor are capable of mutual co-suppression of each other by the formation of a heterodimer. Disclosed here is the fact that the TR2 orphan receptor represses androgen receptor by the formation of a heterodimer. In a similar manner, mutual suppression is demonstrated between the TR2 receptor and the estrogen receptor, also by heterodimer formation. Disclosed here is the fact that the TR4 orphan receptor and the estrogen receptor are capable of mutual co-suppression by the formation of a heterodimer. These observations make possible new approaches to the modulation of sex hormone receptors and new approaches for blocking sex hormone activity.

The transcriptional activity of estrogen receptor (ER), known to be dramatically altered during different stages of mammary gland development as well as during transformation to malignancy, is highly modulated by the character and amount of coregulator proteins present in the cells. Disclosed herein, TR2 orphan receptor (TR2), a member of the nuclear receptor superfamily without identified ligands, is co-expressed with ER in the mammary epithelial cells and the breast cancer cell lines and suppresses ER-mediated transcription through direct protein-protein interaction. Also disclosed herein an interaction blocker, ER-#6 (aa 312-340), can mediate the TR2 interaction. Also disclosed administration of antisense TR2 or ER-#6 resulted in an enhancement of ER transcriptional activity in MCF7 cells indicated that endogenous TR2 normally suppresses ER-mediated signaling. To gain insights into the molecular mechanism by which TR2 suppresses ER, it was found that TR2 interrupted ER DNA binding through disrupting the homodimerization of ER by the formation of ER-TR2 heterodimers. The suppression of ER transcription by TR2, consequently, caused the inhibition of estrogen-induced cell growth and G1-S transition in MCF7 cells.

The human TR4 orphan receptor (TR4) is a member of the steroid/thyroid hormone receptor superfamily. It has been documented that the TR4 may bind as a homodimer to a DNA response element containing two direct repeats of the AGGTCA consensus motif.

1. Receptor DNA recognition

Differential recognition of target genes by the steroid/thyroid hormone members is determined by at least three properties: protein-DNA interactions, protein-protein interactions, and protein environment. For the protein-DNA interaction, DNA-binding domains of family members selectively interact with HREs, which are structurally related but functionally distinct. Based on the zinc finger model, the proximal box in the DNA-binding domain of receptor proteins may determine target HRE specificity (Umesono, K. et al. (1989) Cell 57, 1139-1146). Consequently, the TR4 can be grouped into members of the estrogen receptor subfamily, recognizing direct repeats of the hexameric consensus motif AGGTCA (Umesono, K. et al. (1989) Cell 57, 1139-1146). Thus, it has been demonstrated that the TR4 may bind to tandem repeats, including the +55 region of the simian virus (SV) 40 major late promoter (Young, W. et al. (1997) J. Biol. Chem. 272, 3109-3116), the fifth intron of the ciliary neurotrophic factor receptor (Lee, H. et al. (1995) J. Biol. Chem. 270, 30129-30133), CRBPII and RARβ genes, respectively (Lee, Y. et al. (1998) J. Biol. Chem. 273, 13437-13443). In addition, the TR4 has been shown to bind as a homodimer to direct repeats of the AGGTCA core sequence spaced by one (DR1) or more nucleotides (Hirose, T. et al. (1994) Mol. Endocrinol. 8, 1667-1680).

2. TR2

The human TR2 orphan receptor (TR2), a member of the nuclear hormone receptor superfamily, was cloned from human testis and prostate cDNA libraries and has no previously identified ligand(s) (Chang, C. et al. (1989) Biochem. Biophys. Res. Commun. 165, 735-41; Chang, C. et al. (1988) Biochem. Biophys. Res. Commun. 155, 971-7). Its cDNA encodes a protein of 603 amino acids with a calculated molecular mass of 67 kilo-daltons [Chang C. et al. (1988) Biochem Biophys Res Commun 155:971-977; Chang C. et al. (1988) Biochem Biophys Res Commun 165:735-741]. TR2 is mapped to locate on chromosome 12q22 (Lin, D. et al. (1998) Endocrine 8, 123-34), known to be frequently deleted in various tumors, including testicular and ovarian germ cell tumors (Faulkner, S. et al. (2000) Gynecol. Oncol. 77, 283-8; Murty, V. et al. (1996) Genomics 35, 562-70). Four RNA isoforms, TR2-5, -7, -9, and -11, have been identified. While TR2-11 encodes the full-length receptor, TR2-5, -7, and -9 encode truncated receptors with distinct deletions of ligand-binding domains (LBD) (Chang, C. et al. (1989) Biochem. Biophys. Res. Comun. 165, 735-41). TR2 has high homology with TR4, which places them in a unique subfamily within the nuclear hormone receptor superfamily (Chang, C. et al. (1994) Proc. Natl. Acad. Sci. USA 91, 6040-4). TR2 is evolutionarily conserved among species from primitive creatures to mammalians, including sea urchin, rainbow trout, axolotl, xenopus, drosophila, mouse, and human (Chang, C. et al. (1989) Biochem. Biophys. Res. Commun. 165, 735-41, Chang, C. et al. (1988) Biochem. Biophys.Res. Commun. 155, 971-7; Kontrogianni-Konstantopoulos, A. et al. (1996) Dev. Biol. 177, 371-82; Le Jossic, C. et al. (1998) Biochem Biophys Res Commun 245, 64-9). The facts that TR2 is broadly expressed in many tissues throughout development starting at as early as mid-gestation stage (Lee, C. et al. (1996) Mol. Reprod. Dev. 44, 305-14; Young, W. et al. (1998) J. Biol. Chem. 273, 20877-85; Lee, H. et al. (1996) J. Biol. Chem. 271, 10405-12; Lin, T. et al. (1995) J. Biol. Chem. 270, 30121-8) and that drosophila with null mutations of DHR78 nuclear receptor, a homolog of human TR2, is lethal at the third-instar larval stage with severe defects in ecdysteroid-triggered metamorphosis (Fisk, G. et al. (1998) Cell 93, 543-55) are consistent with the biological importance of TR2 being involved in the development process. It has been emphasized that with prominent expression throughout the active proliferating zones of the neural areas and the sensory nerve-targeted organs and the testes during development, TR2 may exert an important role in the early development of the nervous system and the male reproductive system (Lee, C. et al. (1996) Mol. Reprod. Dev. 44, 305-14; Young, W. et al. (1998) J. Biol. Chem. 273, 20877-85; Lee, H. et al. (1996) J. Biol. Chem. 271, 10405-12; Lin, T. et al. (1995) J. Biol. Chem. 270, 30121-8). Also, it has been shown that TR2 is primarily expressed in the mouse testis, particularly in the developing germ cells, indicating a role of TR2 in spermatogenesis (Lee, C. et al. (1996) Mol. Reprod. Dev. 44, 305-14; Lee, C. et al. (1995) Genomics 30, 46-52). The expression of TR2 has been detected widely in the male reproductive system including testis, prostate, and seminal vesicle [Chang C. et al. (1988) Biochem Biophys Res Commun 155:971-977; Chang C. et al. (1988) Biochem Biophys Res Commun 165:735-741; Lee C. et al. (1996) Mol Reprod Dev 44:305-314]. TR2 is also relatively highly expressed in prostate cancer tissue and cell lines [Chang C. et al. (1988) Biochem Biophys Res Commun 155:971-977; Chang C. et al. (1988) Biochem Biophys Res Commun 165:735-741; Lee C. et al. (1996) Mol Reprod Dev 44:305-314; Hu Y -C. et al. (2002) J Biol Chem 277:33571-33579].

In cell line models, information regarding TR2 function, in terms of transcription activity, has been demonstrated by many studies. TR2 functions as a transcription factor that binds to its consensus response element (AGGTCA) in a direct repeat (DR) orientation (AGGTCA(n)ₓAGGTCA, x=1-6) (Lin, T. et al. (1995) J. Biol. Chem. 270, 30121-8). New TR2 target genes are continually being discovered, such as cellular retinol-binding protein II (CRBPII), retinoic acid receptor β (RARβ, SV40, erythropoietin, histamine H1 receptor, muscle-specific aldolase A, and ciliary neurotrophic factor receptor (CNTFR) (Young, W. et al. (1998) J. Biol. Chem. 273, 20877-85; Lee, H. et al. (1995) J. Biol. Chem. 270, 5434-40; Lin, T. et al. (1995) J. Biol. Chem. 270, 30121-8; Lee, H. et al. (1995) J. Biol. Chem. 270, 5434-40; Lee, H. et al. (1995) J. Biol. Chem. 270, 30129-33; Chang, C. et al. Mol. Cell Biochem. 189, 195-200; Lee, H. et al. (1999) Mol Cell Biochem 194, 199-207), suggesting that TR2 has a broad range of biological functions. In terms of the regulation of TR2 expression, TR2 can be induced during neuronal differentiation in P19 embryonic carcinoma cells stimulated by ciliary neurotrophic factor (CNTF). In return, TR2 activates its target gene, CNTFR, expression which mediates CNTF signaling and is required for the motor neuron development (Young, W. et al. (1998) J. Biol. Chem. 273, 20877-85; DeChiara, T. et al. (1995) Cell 83, 313-22). These can provide a linkage between TR2 and neurogenesis. The tumor suppressor genes, p53 and Rb, that induce cell cycle arrest can down-regulate TR2 expression in cells after ionizing radiation and in cells overexpressing p53 or Rb (Mu, X. et al. (2000) J. Biol. Chem. 275, 23877-83; Lin, D. et al. (1996) J. Biol. Chem. 271, 14649-52). TR2 can then go through a feed-back control mechanism to induce HPV-16 E6 and E7 target gene expression that are known to enhance the P53 protein degradation and inactivate the Rb function, respectively (Mu, X. et al. (2000) J. Biol. Chem. 275:23877-83; Collins, L. et al. (2001) J Biol Chem 276:27316-21). TR2 is, therefore, thought to be involved in cell cycle regulation.

In addition to functioning as a transcription regulator, TR2 can modulate other signaling via different mechanisms. For example, TR2 suppresses RXR- and RXR/RAR-mediated transcription by binding to the same DNA response element (DRE) with a higher binding affinity (Lin, T. et al. (1995) J. Biol. Chem. 270, 30121-8) and represses thyroid receptor α/RXR signaling by competing for limited amounts of DREs (Chang, C. et al. Mol. Cell Biochem. 189, 195-200). TR2 can also exert its suppressive effects via the recruitment of class I and class II histone deacetylases (HDAC) (Franco, P. et al. (2001) Mol Endocrinol 15, 1318-28).

TR2 can be down regulated by the tumor suppressor genes p53 and Rb in cells after ionizing radiation and in cells over-expressing p53 and Rb. TR2 is also thought to control the expression of p53 and Rb through the regulation of human papillomavirus 16 E6/E7 genes [Lin D -L, (1996) J Biol Chem 271:14649-14652; Mu X. M. et al. (2002) J Biol Chem 275:23877-23883]. TR2 is, therefore, thought to be involved in cell cycle regulation and tumorgenesis.

Androgen and AR play an essential role in prostate proliferation and prostate cancer progression. Disclosed herein TR2 can modulate AR-mediated transactivation and target gene expression possibly through TR2 and AR interaction in human prostate cancer PC-3 cells.

3. TR4

The human testicular receptor 4 (TR4) was originally isolated from testes, prostate, and brain cDNA libraries by degenerative polymerase chain reaction cloning (Chang, C. et al. (1994) Proc. Natl. Acad. Sci. USA. 91(13), 6040-4). While TR4 shares the structural features of nuclear receptors, no ligand has yet been previously identified and it is therefore considered an orphan receptor.

TR4 directly regulates transcription through binding to a direct repeat (DR) of a AGGTCA core element separated by a variable number of nucleotides. (TR2 and TR4 can bind to AGGTCA direct repeats (DRx; AGGTCA (n)ₓ AGGTCA, x=0-6) (Lee, Y. et al. (1997) J. Biol. Chem. 272, 12215-12220; Young, W. et al. (1997) J. Biol. Chem. 272, 3109-3116; Young, W. et al. (1998) J. Biol. Chem. 273, 20877-20885; Lin, T. et al. (1995) J. Biol. Chem. 270, 30121-30128; Lee, Y. et al. (1998) J. Biol. Chem.273, 13437-13443; Lee, H. et al. (1995) J. Biol. Chem. 270, 5434-5440; Lee, H. et al. (1995) J. Biol. Chem. 270, 30129-30133; Lee, H. et al. (1996) J. Biol. Chem. 271, 10405-10412; and Hanley, K. et al.(1998) J Invest Dermatol 110, 368-375). TR4 functions as a transcriptional activator when bound to the DR separated by four nucleotides (a DR-4 element) (Lee, Y. et al. (1997) J. Biol. Chem. 272(18) 12215-20). However, TR4 functions as a transcriptional repressor when bound to DR-1, DR-2, DR-3, or DR-5 type (Lee, Y. et al. (1998) J. Biol. Chem. 273(22) 13437-43; Lee, H. et al. (1995) J. Biol. Chem. 270(50) 30129-33; Lee, Y. et al. (1999) J. Biol. Chem. 274(23) 16198-205). The differential spacings between the core elements cause TR4 to adopt different conformations and alter the ability of TR4 to interact with coregulators (Lee, Y. et al. (1999) J Biol. Chem. 274(23) 16198-205). Consistent with its neuronal localization, TR4 also induces the transcription of the cytokine receptor, which is a ciliary neurotrophic factor receptor (Young, W. et al. (1997) J. Biol. Chem. 272(5) 3109-16).

In addition to direct transcriptional regulation, TR4 can also modulate other nuclear receptors' transactivation. Previous studies have indicated that TR4 can compete for binding to the hormone response elements of retinoic acid receptor (RAR), retinoid X receptor (RXR) (Lee, Y. et al. (1998) J. Biol. Chem. 273(22) 13437-43) and vitamin D receptor (VDR) (Lee, Y. et al. (1999) J. Biol. Chem. 274(23) 16198-205) to suppress RAR/RXR- or VDR-mediated transcription. TR4 can also inhibit peroxisome proliferator activated receptor alpha (PPARα) induced transactivation by competitive binding to PPAR response elements and through competition for coactivators such as RIP140 (Yan, Z. et al. (1998) J. Biol. Chem. 273(18) 10948-57). The AR-TR4 interaction could then result in the mutual suppression of AR- or TR4-mediated transcription (Lee, Y. et al. (1999) Proc. Natl. Acad Sci. USA. 96(26) 14724-9). Previous reports have linked TR4 function to neurogenesis (Young, W. et al. (1997) J. Biol. Chem. 272(5) 3109-16) and spermatogenesis (Lee, C. et al. (1998) J. Biol. Chem. 273(39) 25209-15). TR4 has been demonstrated to suppress many other receptors' transactivation, such as VDR, RAR, RXR, and PPAR (Lee, Y. et al. (1998) J. Biol. Chem. 273(22) 13437-43; Lee, Y. et al. (1999) J. Biol. Chem. 274(23) 16198-205; Yan, Z. et al. (1998) J. Biol. Chem. 273(18) 10948-57.

The suppression mechanism for these receptors' transactivation has been demonstrated through the competition of TR4 with those receptors' ability to bind their hormone response elements.

The human TR4 cDNA shares structural homology with members of the steroid hormone receptor superfamily (Chang, C. et al. (1994) Proc. Natl. Acad. Sci. USA. 91, 6040-6044). The TR4, also named as TAK1 (Hirose, T. et al. (1994) Mol. Endocrinol. 8, 1667-1680), is most closely related to the previously identified TR2 orphan receptor (Chang, C. et al. (1988) Biochem. Biophys. Res. Commun. 155, 971-977; Chang, C. et al. (1989) Biochem. Biophys. Res. Commun. 165, 735-741; Gronemeyer, H. et al. (1995) Protein Profile 2, 1173-1308), forming subfamily within the superfamily of steroid receptors. Recently, the TR4 was designated as the TR2β, while the TR2 orphan receptor was referred to as TR2α (Mangelsdorf, D. J. et al. (1995) Cell 83, 841-850). The mouse TR4 cDNA has been cloned from mouse testis by reverse transcription-PCR (Young, W. et al. (1997) J. Biol. Chem. 272 3109-3116). Subsequently, the human TR4 gene has been mapped to chromosome 3p24.3 (Lin, D. et al. (1998) Endocrine 8 123-134).

TR4encodes a 67 kDa protein (Chang, C. et al. (1994) Proc. Natl. Acad. Sci. USA 91, 6040-6044) of 615 amino acids with a very high homology with TR2 orphan receptor (Chang, C. et al. (1988) Biochem. Biophys. Res. Commun. 155:971-977; Chang, C. et al. (1989) Biochem. Biophys. Res. Commun. 165:735-741). The P-box sequence of the DNA binding domain (DBD), TR4 is classified as a member of the estrogen receptor and thyroid hormone receptor subfamily, which can recognize the hormone response elements (HREs) composed of the AGGTCA motif. Examples of HREs with this motif include those of the retinoic acid receptor (RARE), retinoid X receptor (RXRE) (Lee, Y. -F., et al., (1998) J. Biol. Chem. 273, 13437-13443), thyroid hormone receptor (T₃RE) (Lee, Y. et al. (1997) J. Biol. Chem. 272, 12215-12220) and vitamin D receptor (VDRE)¹. In this case, TR4 may interfere with other steroid hormone pathways by binding to the same HREs. Several in vitro studies have demonstrated that TR4 acts as a regulator of various steroid/thyroid hormone pathways (Lee, Y. et al. (1997) J. Biol. Chem. 272, 12215-12220; Young, W. et al. (1997) J. Biol. Chem. 272, 3109-3116; Lee, H. et al. (1995) J. Biol. Chem. 270, 30129-30133; Young, W. et al. (1998) J. Biol. Chem. 273, 20877-20885).

In situ hybridization analysis shows that TR4 is highly expressed in adult mouse brain especially in the regions in which cells undergo active proliferation and in the granule cells of the hippocampus and cerebellum (Chang, C. et al. (1994) Proc. Natl. Acad. Sci. USA. 91(13), 6040-4). It has been demonstrated that TR4 inhibits the retinoic acid (RA) pathway that is highly involved in the development of the nervous system (Young, W. et al. (1998) J. Biol. Chem. 273, 20877-20885). In contrast, TR4 enhanced the transactivation activity of the ciliary neurotrophic factor receptor (CNTFR) gene, whose expression pattern is restricted to nervous tissues and is highly similar to that of TR4, via binding to CNTFR-DR1 (Young, W. et al. (1997) J. Biol. Chem. 272, 3109-3116). It was found that treatment of cells with RA would increase TR4 amounts at both RNA and protein levels (Lee, Y. et al. (1998) J. Biol. Chem. 273, 13437-13443). The TR4 increase was also observed in CNTF-treated mouse P19 teratocarcinoma cells (Young, W. et al. (1998) J. Biol. Chem. 273, 20877-20885). The data from both in situ and in vitro studies suggest that TR4 may be involved in the regulation of differentiation of neuron cells.

In situ hybridization analysis has demonstrated that TR4 is expressed in a complex spatiotemporal pattern, including, the central neural system (habenula, hippocampal pyramidal cell, and granule cells of both hippocampus and cerebellum) and peripheral organs (most abundantly expressed in spennatocytes of testis, with lower amounts in adrenal cortex, spleen, thyroid, prostate, and pituitary gland) (Chang, C. et al. (1994) Proc. Natl. A cad. Sci. USA 91:6040-6044; Hirose, T. et al. (1994) Mol. Endocrinol. 8:1667-1680; Young, W. J. et al. (1997) J: Biol. Chem. 272:3109-3116; Van Schaick, H. S. A. et al. (2000) Mol. Brain Res. 77:104-110). In the development of neurons, TR4 transcripts were detected throughout the neural tube at early stages of embryo development, and were subsequently restricted to the regions where cells were rapidly proliferating in the later stages of the embryo (Young, W. et al. (1997) J. Biol. Chem. 272, 3109-3116). Consistent with in situ analysis of mouse embryos, the TR4 transcripts were expressed higher in the S-phase than in G1 and G2/M phases, determined by testing of elutriated P19 cell fractions.

In addition, the expression of the TR4 transcripts occurs widely in many mouse tissues, including the central nervous system and peripheral organs such as the adrenal gland, spleen, thyroid gland, and prostate (Yoshikawa, T. et al. (1996) Endocrinol. 137, 1562-1571; Young, W. et al. (1997) J. Biol. Chem. 272, 3109-3116). These data are consistent with TR4 playing a role in neurogenesis and neuronal maturation.

TR4 can function as a transcriptional factor to regulate many signal transduction pathways. For example, it has been shown that TR4 can function as repressor to suppress the retinoic acid receptor (RAR), retinoid X receptor (RXR), Vitamin D receptor, androgen receptor (AR), and estrogen receptor mediated transactivation (Lee, Y. F et al. (1998) J: Biol. Chem. 273:13437-13443; Lee, Y. F. et al. (1999) J. Biol. Chem. 274:16198-16205; Lee, Y. F. et al. (1999) Proc. Natl. Acad. Sci. USA 96:14724-14729; Shyr, C. R. et al. (2002) J. Biol. Chem. 277:14622-14628). A recent report (Lee, H. J. et al. (2001) Biochem. Biophys. Res. Commun. 285:1361-1368) demonstrated that TR4 could suppress the expression of the steroid 21-hydroxylase gene, which belongs to the cytochrome P450 superfamily, and is one of the key enzymes in biosynthesis of adrenal steroid hormones, leading to the production of cortisol and aldosterone. On the other hand, TR4 also can function as enhancer to induce the ciliary neurotrophic factor receptor-a and thyroid hormone receptor (TR) (Young, W. J. et al. (1997) J: Biol. Chem. 272:3109-3116; Lee, Y. F. et al. (1997) J. Biol. Chem. 272:12215-12220). A recent report (Koritschoner, N. P. et al. (2001) Cell Growth Differ. 12, 563-572) has shown that the TR4 is an important regulator of myeloid progenitor cell proliferation and development. The hormone response element for the TR4 is AGGTCA with a variety of direct repeats (DRs) (Lee, Y. F. et al. (1999) Proc. Natl. Acad. Sci. USA 96:14724-14729; Young, W. J. et al. (1998) J. Biol. Chem. 273:20877-20885).

4. Androgen Receptor

The androgen receptor (AR) is a ligand inducible transcription regulator that can activate or repress its target genes by binding to its hormone response elements (HRE) as a homodimer. The AR consists of four major functional domains including a ligand binding domain (LBD), and two activation functions (AF) residing in the N-terminal (AF-1) and the C-terminal end of the LBD (AF-2) respectively.

Androgen receptor belongs to a superfamily of steroid hormone receptors was first subcloned in 1988 (Chang, 1988). It contains a N-terminal transactivation domain (variable), a central DNA binding domain (DBD) (conserved) and a C-terminal ligand binding domain (LBD) (conserved) (Umesono, 1995). By forming a homodimer and taking into account of the ligand and coregulators, the androgen receptors interact and regulate the transcription of numerous target genes (Ing, 1992; Schulman, 1995; Beatp, 1996; Yeh, 1996; Glass, 1997, Shibata, 1997). Androgen is the strongest ligand of the androgen receptor. However, it is not the only ligand. Estradiol has been found to activate androgen receptor transactivation through the interaction with androgen receptor (Yeh, 1998). Besides, androgen and androgen receptor do not only act in male. The increasing evidence has displayed that the androgen and androgen receptor (AR) may also play important role in female physiological processes, including the process of folliculogenesis, the bone metabolism and the maintenance of brain functions (Miller, 2001).

Androgen is the most conspicuous amount of steroid hormone in ovary (Risch H A, 1998). The concentrations of testosterone and estradiol in the late-follicular phase when estrogens are at their peak are 0.06-0.10 mg/day and 0.04-0.08 mg.day respectively (Risch HA, 1998). The ratio of androgens versus estrogens in the ovarian veins of postmenopausal women is 15 to 1 (Risch, 1998; Doldi N, 1998). Androgen receptor is expressed dominantly in granulosa cells of ovary (Hiller S G, 1992; Hild-Petito S, 1991). With the overproduction of ovarian androgen, women with polycystic ovarian syndrome suffered from impairment of ovulatory function which is characterized with the increasing number of small antral follicles, but arrest in grafian follicles development (Kase, 1963; Futterweit W, 1986; Pache T D, 1991; Spinder T, 1989; Spinder T, 1989; Hughesdon P E, 1982). This symptom has suggested that AR may play a proliferative role in early folliculogenesis but turn to inhibitory effect in late folliculogenesis. The recent studies conducted in animals have supported this hypothesis (Harlow C R, 1988; Hillier S, 1988; Weil S, 1998; Vendola K, 1998; Weil S, 1999; Vendola K, 1999). Administration of hihydroxytestosterone (DHT) in rhesus monkeys has increased the number of primary, preantral and small antral follicles. Since DHT is the metabolite of testosterone and cannot be aromatized, the result suggested the proliferative effect was through AR system (Vendola K, 1999).

Ligand-dependent transcriptional activation of AR is mediated by the COOH-terminal domain that includes the ligand binding domain and a activation function domain (AF-2) [O'Malley B. (1990) Mol Endocrinol 4:363-369]. Crystallographic studies show that ligand-bound steroid receptors undergo a conformational change in the AF-2 core motif. The ligand-induced conformational change presumably recruits coregulators. Many AR coregulators, such as SRC-1, TIF-2, CBP/P300, ARA70, ARA54, ARA55, ARA24, ARA160, p/CIP/ACTR/AIB1, Rb, and NCoA-1 have been identified [Heinlein C. A. et al. (2002) Endorcr Rev 23:175-200].

5. Estrogen Receptor

Estrogen receptors (ERs), including ERα and ERβ, belong to nuclear hormone receptor superfamily and mediate estrogen actions in regulation of cell growth and differentiation, particularly in mammary glands and uterus in females (see reviews in (Nilsson, S. et al. (2001) Physiol Rev 81, 1535-65; Couse, J. et al. (1999) Endocr Rev 20, 358-417)). The proliferation of mammary glands is mainly dependent on estrogen stimulation; however, the proliferating epithelial cells detected in terminal end buds (TEBs) at the tip of elongating ducts in mammary glands are usually ER-negative (Zeps, N. et al. (1998) Differentiation 62, 221-6; Jensen, E. et al. (2001) Proc Natl Acad Sci USA 98, 15197-202; Sapino, A. et al. (1990) J Histochem Cytochem 38, 1541-7). Despite the unclear role of ER in this process, in mice with a homozygous disruption of ER genes, the mammary glands remain undeveloped as demonstrated by the lack of TEBs and alveolar structures, even though the serum estrogen levels are 10 times higher than those in wild-type mice (Couse, J. et al. (1995) Biochem Soc Trans 23, 929-35; Bocchinfuso, W. et al. (1999) Cancer Res 59, 1869-76). This indicates a role of ER in the growth of mammary glands. Also, the fact that more than two thirds of breast cancers from patients are ER-positive and benefit from antiestrogen or ovariectomy therapies, strengthens the ER involvement in stimulation of cell growth in mammary glands in response to estrogen (Group, E. B. C. T. C. (1998) Lancet 351, 1451-67).

Estrogen receptors (ER) that play many essential roles for the growth in female reproductive tissues are encoded by two distinct genes, ERα and ERβ (Kuiper, G. et al. (1996) Proc. Natl. Acad. Sci. USA. 93(12):5925-30). It has been demonstrated that ERα and ERβ can form heterodimers (Cowley, S. et al. (1997) J. Biol. Chem. 272(32):19858-623), and ERα was able to directly bind to TR, RAR, RXR (Lee, S. et al. (1998) Mol. Endocrinol. 12(8): 1184-92), short heterodimer partner (SHP) (Seol, W. et al. (1998) Mol. Endocrinol. 12(10):1551-7; Johansson, L. et al. (1999) J. Biol. Chem. 274(1):345-53), and ERβcx (Ogawa, S. et al. (1998) Nucleic Acids Res. 26(15):3505-12). ERα-TR and ERα-RXR heterocomplexes moderately enhance ER-mediated transcription in transient transfection experiments with CV-1 cells. In contrast, RAR repressed ER-mediated transactivation (Lee, S. et al. (1998) Mol. Endocrinol. 12(8):1184-92). The SHP inhibits ER transcriptional activity by preventing coactivator binding to ER (Johansson, L. et al. (1999) J. Biol. Chem. 274(1):345-53) and ERβcx inhibits ER transactivation by preventing ER binding to DNA (Ogawa, S. et al. (1998) Nucleic Acids Res. 26(15):3505-12). It is demonstrate herein that TR4 also inhibits ER transcriptional activity in lung cancer H1299 cells and in breast cancer MCF-7 cells. Further studies indicate that TR4 can suppress ER function via protein-protein interaction that results in the interruption of ER-ER homodimerization and in preventing ER binding to its estrogen response element (ERE). The analysis of ERα KO mice indicated that ERα can play important in vivo functions, such as the growth of the adult female reproductive tract and mammary gland, the regulation of gonadotropin gene transcription, mammary neoplasia induction, and sexual behaviors. Surprisingly, ERα also play important roles in spermatogenesis and sperm function (see review (Couse, J. et al. (1999) Endocr. Rev. 20(3), 358-417)).

6. Protein Domains

A variety of proteins are discussed herein, including TR2, TR4, AR, and ER. It is understood that these proteins have a variety of functional domains which are herein disclosed (Table 3)

TABLE 3

| | TR2 | TR4 | AR | ER |
|---|---|---|---|---|
| DBD | 88-196 | 134-201 | 486-651 | 123-340 |
| LBD | 179-603 | 224-615 | 666-918 | 499-595 |
| | | | | 282-595 |
| | | | | 312-595 |
| Dimerization | | | | |
| Domain | | | | |
| F domain | | | | 552-595 |
| N-terminal | 1-112 | 1-133 | 34-560 | 1-165 |
| domain | | | | |

Table 4 shows the various interactions which have been tested. Furthermore, the domains are interchangeable. For example, it is understood what the F domain interacts with, and it can be predicted, for example that if it binds TR2 in a particular spot, it would be expected that it could bind TR4 in a homologous domain.

TABLE 4

| | | | | | | ER | ER | | | |
|---|---|---|---|---|---|---|---|---|---|---|
| | | AR | AR | AR | | LBD | LBD | ER | F | ER |
| | AR | LBD | DBD | N-T | ER | 312-595 | 282-595 | DBD | Domain | N-T |
| TR4 | + | + | + | + | + | | + | | | |
| TR4 | + | | | | + | | + | | | |
| LBD | | | | | | | | | | |
| TR4 | | | | | | | | | | |
| DBD | | | | | | | | | | |
| TR4 | | | | | | | | | | |
| N-T | | | | | | | | | | |
| TR2 | + | + | | | + | + | + | − | + | − |
| TR2 | | | | | − | | | | | |
| LBD | | | | | | | | | | |
| TR2 | | | | | + | | | | | |
| DBD | | | | | | | | | | |
| TR2 | | | | | − | | | | | |
| N-T | | | | | | | | | | |

Table 5 shows the effect of the various interactions on the transcription activity of a particular transactivator. These relationships shown have been tested and it is understood that these relationships and effects indicate other effects. For example, as TR4 down regulates AR and AR down regulates TR4 it can be indicated since TR2 down regulates AR that AR also down regulates TR2.

TABLE 5

| | | Activity | | |
|---|---|---|---|---|
| | AR | ER | TR4 | TR2 |
| Regulator | | | | |
| TR4 | Down | Down | | |
| TR2 | Down | Down | | |
| AR | | | Down | |
| ER | | | Down | Down |

7. Interactions with TR2

Disclosed herein TR2 can interact with a number of proteins. These interactions can alter TR2 transcriptional activation activity as well as altering the transcriptional activation activity of the proteins TR2 interacts with. Disclosed herein TR2 interacts with ER.

a) Interaction Between ER and TR2

Using an in vitro GST pull-down assay and an in vivo mammalian two-hybrid system, ER and TR2 were found to heterodimerize. In the in vitro GST pull-down assay, it was further found that the LBD fragments encoding aa 312-595 or aa 312-340 of ER could also bind to TR2.” In the in vivo mammalian two-hybrid system, E2 could promote the dimerization between ER LBD (aa 282-595) and TR2.

The significance of TR2/ER dimerization lies first in the fact that it prevented ER from binding to its target DNA and thus repressed ER transactivation. TR2 LBD was sufficient to prevent ER from binding to its target DNA. When cells were treated with E2, ER was induced to transactivate several downstream genes. Overexpression of TR2 in the same cells could block the ER-induced expression of these downstream genes in a dose-dependent manner. Therefore, the present invention is also directed at new treatment strategies for ER-related diseases such as breast cancer by modulating TR2 levels to enhance or repress ER's transactivation activity. If a disease or clinical condition is associated with an increase of ER-mediated transactivation, the disease or clinical condition can be cured or controlled by increasing the TR2 level. If a disease or clinical condition is associated with a decrease of ER-mediated transactivation, the disease or clinical condition can be cured or controlled by decreasing the TR2 level. The full length TR2 could repress AR's transactivation activity. Any method or agent that can increase or decrease TR2 levels in human bodies are candidates for treating ER-related diseases. Since the LBD of TR2 could prevent ER from binding to its target DNA as well, any truncated form of TR2 that retains the LBD or any chimeric protein that contains the TR2 LBD can also be used when treatment requires repressing ER's transactivation activity.

ER transactivation induced in any manner, including that by constitutively active ER, can be repressed by TR2.

The compositions and methods make it possible to screen for drugs for ER-related diseases by testing a compound's effect on TR2 level. If a compound can increase or decrease the level of TR2 in a cell, then it can be selected for further testing for treatment of ER-related diseases. The screening method can measure TR2 level directly. It can also measure TR2 level indirectly, for example, through any reporter system that measures the increase or decrease of TR2 transactivation. Examples of such reporter systems are described below.

The significance of TR2/ER heterodimerization lies second in the fact that it repressed TR2 transactivation activity in an ER dose dependent manner. Therefore, human clinical conditions such as hair loss that can relate to TR2 transactivation activity can be controlled through modulating ER levels in human. If a disease or clinical condition is associated with an increase of TR2-mediated transactivation, the disease or clinical condition can be cured or controlled by increasing the ER level. If a disease or clinical condition is associated with a decrease of TR2-mediated transactivation, the disease or clinical condition can be cured or controlled by decreasing the ER level. Any method or agent that can change ER levels in human bodies are treatment candidates.

Since ER LBD could also bind to TR2, any truncated form of ER that retains the LBD or any chimeric protein that contains the ER LBD are also potential treatment candidates when treatment requires repressing TR2 transactivation.

The disclosed compositions and methods also makes it possible to screen for drugs for TR2-related diseases by testing a compound's effect on ER level. If a compound can increase or decrease the level of ER in a cell, then it can be selected for further testing for treatment of TR2-related diseases. The screening method can measure ER level directly. It can also measure ER level indirectly, for example, through any reporter system that measures the increase or decrease of ER transactivation.

In addition, TR2 can form heterodimers with either TR4 or AR as well. Co-repression exists in these two dimers as well.

b) TR2-ER Interaction

TR2 interacts with ER. This interaction causes a decrease in ER activated transcription and a decrease in ER activated transcription.

The TR2-ER interaction occurs in a region of the ER protein. ER-#2 (aa 123-340), ER-#3 (aa 312-595), but not ER-#1 (aa 1-165) and ER-#4 (aa 552-595) can interact with TR2 in the presence or absence of E2. Furthermore, ER-#6 (aa 312-340), the overlapping region between GST-ER-#2 and -#3, but not ER-#5 (aa 123-312), showed positive interaction with TR2, indicating that the ER-#6 domain is responsible for this interaction.

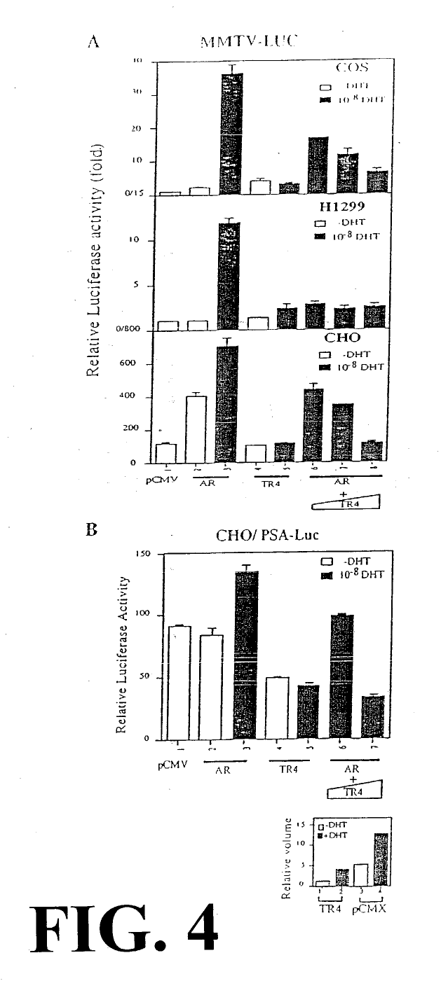

On the other hand, three GST-fused TR2 fragments, TR2-#1, -#2, and -#3, corresponding to N-terminus (aa 1-112), DBD (aa 88-196), and LBD (aa 179-603), respectively, were also examined to locate the ER-binding site. As shown in FIG. 4B, GST-TR2-#2, but not GST-TR2-#1 or -#3, was responsible for binding to ER.

The interaction between TR2 and ER can repress ER mediated transcription activation at ER induced promoters as well as TR2 mediated transcription activation from TR2 induced promoters.

c) Interactions Between AR and TR2

Using an in vitro GST pull-down assay and an in vivo mammalian two-hybrid system, AR and TR2 were found to heterodimerize. TR2 was found to suppress AR mediated transactivation in PC-3 prostate cancer cells.

The significance of TR2/AR dimerization lies first in the fact that it prevented AR from binding to its target DNA and thus repressed AR transactivation. TR2 in prostate cancer cells, such as LNCaP or PC-3 cells could block the AR-induced expression of these downstream genes in a dose-dependent manner. Therefore, the disclosed compositions and methods are also directed at new treatment strategies for AR-related diseases such as prostate cancer by modulating TR2 levels to enhance or repress AR's transactivation activity. If a disease or clinical condition is associated with an increase of AR-mediated transactivation, the disease or clinical condition can be cured or controlled by increasing the TR2 level. If a disease or clinical condition is associated with a decrease of AR-mediated transactivation, the disease or clinical condition can be cured or controlled by decreasing the TR2 level. The full length TR2 could repress AR's transactivation activity. Any method or agent that can increase or decrease TR2 levels in human bodies are candidates for treating ER-related diseases. Since TR2 could prevent AR from binding to its target DNA as well, any TR2 as well as truncated forms of TR2 that retain the ability to interact with AR or any chimeric protein that contains the TR2, TR2 variant, or TR2 fragment can also be used when treatment requires repressing AR's transactivation activity.

AR transactivation induced in any manner, including that by constitutively active AR, can be repressed by TR2.

The compositions and methods make it possible to screen for drugs for AR-related diseases by testing a compound's effect on TR2 level. If a compound can increase or decrease the level of TR2 in a cell, then it can be selected for further testing for treatment of AR-related diseases. The screening method can measure TR2 level directly. It can also measure TR2 level indirectly, for example, through any reporter system that measures the increase or decrease of TR2 transactivation. Examples of such reporter systems are described below.

The significance of TR2/AR heterodimerization lies second in the fact that it repressed TR2 transactivation activity in an AR dose dependent manner. Therefore, human clinical conditions such as hair loss that can relate to TR2 transactivation activity can be controlled through modulating AR levels in human. If a disease or clinical condition is associated with an increase of TR2-mediated transactivation, the disease or clinical condition can be cured or controlled by increasing the AR level. If a disease or clinical condition is associated with a decrease of TR2-mediated transactivation, the disease or clinical condition can be cured or controlled by decreasing the AR level. Any method or agent that can change AR levels in human bodies are treatment candidates.

The disclosed compositions and methods also makes it possible to screen for drugs for TR2-related diseases by testing a compound's effect on AR level. If a compound can increase or decrease the level of AR in a cell, then it can be selected for further testing for treatment of TR2-related diseases. The screening method can measure AR level directly. It can also measure AR level indirectly, for example, through any reporter system that measures the increase or decrease of AR transactivation.

d) TR2-ER Interaction

TR2 interacts with ER. This interaction causes a decrease in TR2 activated transcription and a decrease in ER activated transcription.

8. Interactions with TR4

Disclosed herein TR4 can interact with a-number of proteins. These interactions can alter TR4 transcriptional activation activity as well as altering the transcriptional activation activity of the proteins TR4 interacts with. Disclosed herein TR4 interacts with AR and ER.

a) Interaction Between AR and TR4

TR4 interacts with AR. All domains of AR can interact with TR4, including the LBD and the DBD. The interaction between TR4 and AR can repress TR4 mediated transcription activation at TR4 induced promoters as well as AR mediated transcription activation at AR induced promoters.

By using techniques, like a yeast two-hybrid system described below, androgen receptor has been found to heterodimerize with TR4 orphan receptor. Such dimerization has been confirmed in vitro by the GST-TR4 fusion protein pull-down assay. The heterodimerization is not DHT-dependent. This heterodimerization suppresses activity of these two transcriptional factors. Thus each of these transcriptional factors is capable of suppressing the activity of the other.

The heterodimerization between the androgen receptor and TR4 was also demonstrated by an in vivo system using immunocytofluorescence assay, as described below. Transfected TR4 was mainly located in the nucleus while transfected AR was mainly located in the cytoplasm, in the absence of its cognate ligand DHT. However, when TR4 and AR were co-transfected, the majority of the AR was transported to the nucleus by TR4 even in the absence of DHT.

In a mammalian two-hybrid system and a mammalian one-hybrid system, as described below, the ligand binding domain of TR4 was sufficient for binding to a near full-length AR (amino acids 33-918). The binding was DHT-dependent. The difference between DHT-dependent interaction detected in the mammalian one- or two-hybrid systems and DHT-independent interaction detected in the GST pull-down and the immunocytofluorescence assays, could be due to the involvement of AF-1 ligand-independent interaction in the GST pull-down and the immunocytofluorescence assays, which used the full length of TR4 containing AF-1, vs. AF-2 ligand-dependent interaction in the mammalian one- or two-hybrid system, which used only TR4 ligand binding domain containing AF-2.

The significance of TR4/AR heterodimerization lies first in the fact that such dimerization prevents TR4 from binding to its target DNA and thus repressed TR4-mediated transactivation. This repression was observed in mammalian cells in a reporter assay, as described below. The repression is AR dose-dependent. Therefore, human diseases or clinical conditions such as hepatitis, hepatoma and hair loss that can relate to TR4 transactivation activity can be controlled or cured through modulating AR levels in the subject, such as a human. If a disease or clinical condition is associated with an increase of TR4 transactivation, the disease or clinical condition can be affected by modulating the AR level. If a disease or clinical condition is associated with a decrease of TR4 transactivation, the disease or clinical condition can be controlled by decreasing the AR level. Any method or agent that can change the AR level in human bodies are candidates to treat TR4-related diseases.

Since both the full length and a near full-length AR (amino acid 31 to 918) have been tested and shown to heterodimerize with TR4, it is expected that the AR LBD itself is sufficient to bind to TR4 to repress TR4's transactivation. Therefore, it is expected that any truncated form of AR that retains the LBD or any chimeric protein that contains the AR LBD can also be used to treat modulate levels of TR4 activity.

The present invention makes it possible to screen for drugs for TR4-related diseases by testing a compound's effect on AR level. If a compound can increase or decrease the level of AR in a cell, it can be selected for further testing for treatment of TR4-related diseases. The screening method can measure the AR level directly. It can also measure the AR level indirectly, for example, through any reporter system that measures AR transactivation. Examples of such reporter systems are described below.

The significance of TR4/AR heterodimerization lies secondly in the fact that it also represses AR-mediated transactivation. When cells were treated with DHT, AR was induced to transactivate several downstream genes. Overexpression of TR4 in the same cells could block the AR-induced expression of these downstream genes. Therefore, the present invention is also directed at new treatment strategies for AR-related diseases such as prostate-cancer by modulating TR4 levels to enhance or repress AR's transactivation activity. If a disease or clinical condition is associated with an increase of AR-mediated transactivation, the disease or clinical condition can be cured or controlled by increasing the TR4 level. If a disease or clinical condition is associated with a decrease of AR-mediated transactivation, the disease or clinical condition can be cured or controlled by decreasing the TR4 level. The full length TR4 can repress AR's transactivation activity. Any method or agent that can change TR4 levels in human bodies are candidates for treating AR-related diseases. Since the LBD of TR4 could bind to AR as well, any truncated form of TR4 that retains the LBD or any chimeric protein that contains the TR4 LBD can also be used when treatment requires a repression of AR transactivation.

It is expected that AR transactivation induced in any manner, including that by constitutively active AR, can be repressed by TR4.

The present invention makes it possible to screen for drugs for AR-related diseases by testing a compound's effect on TR4 level. If a compound can increase or decrease the level of TR4 in a cell, then it can be selected for further testing for treatment of AR-related diseases. The screening method can measure TR4 level directly. It can also measure TR4 level indirectly, for example, through any reporter system that measures TR4 transactivation.

b) TR4-ER Interactions

TR4 interacts with ER to repress ER transactivation. The LBD of ER interacts well with TR4 in the presence or absence of E2. The TR4-ER interaction occurs in the LBD of TR4. A further deletion of the C-terminal of LBD (pCMV ΔC-TR4) could not repress ER activity and this indicates that the LBD of TR4 is required for TR mediated ER effects.

A compound that is identified or designed as a result of any of the disclosed methods can be obtained (or synthesized) and tested for its biological activity, e.g., inhibition of TR2, TR4, AR, or ER transcription activity.

Disclosed are methods for inhibiting transcription activity of AR, ER, TR2, or TR4, comprising incubating an inhibitor of heterodimerization between AR and TR4, ER and TR4, and/or ER and TR2, for example.

Disclosed are methods of treating a subject comprising administering to the subject an inhibitor of transcription activity of AR, ER, TR2, or TR4, wherein the inhibitor reduces the heterodimerization between AR and TR4, ER and TR4, and/or ER and TR2, and wherein the subject is in need of such treatment.

9. Methods of Inhibiting AR Transcription Activity

Disclosed are methods of inhibiting AR transcription activity comprising administering a composition, wherein the composition prevents AR transcription activity, wherein the composition is defined as a composition capable of being identified by administering the composition to a system, wherein the system supports AR transcription activity, assaying the effect of the composition on the amount of transcription activity in the system, and selecting a composition which causes a decrease in the amount of AR transcription activity present in the system relative to the system without the addition of the composition.

Also disclosed are methods of inhibiting AR transcription activity comprising administering a composition that binds AR, wherein the composition is TR4 or fragment thereof, or a molecule that competitively competes with TR4 for AR binding.

Disclosed are methods of making a composition capable of inhibiting AR transcription activity comprising admixing a compound with a pharmaceutically acceptable carrier, wherein the compound is identified by administering the compound to a system, wherein the system supports AR transcription activity, assaying the effect of the compound on the amount of AR transcription activity in the system, and selecting a compound which causes a decrease in the amount of AR transcription activity in the system relative to the system without the addition of the compound.

Disclosed are methods of manufacturing an inhibitor to AR transcription activity comprising, a) administering a composition to a system, wherein the system supports AR transcription activity, b) assaying the effect of the composition on the amount of AR transcription activity in the system, c) selecting a composition which cause a decrease in the amount of AR transcription activity present in the system relative to the system with the addition of the composition, and d) synthesizing the composition.

Also disclosed are methods comprising the step of admixing the composition with a pharmaceutical carrier.

Disclosed are cells that further comprising an inhibitor of a AR transcription activity.

10. Methods of Inhibiting TR4 Transcription Activity

Also disclosed are methods of identifying inhibitors of TR4 transcription activity comprising, a) administering a composition to a system, wherein the system supports TR4 transcription activity, b) assaying the effect of the composition on the amount of TR4 transcription activity in the system, and c) selecting a composition which causes a decrease in the amount of TR4 transcription activity present in the system relative to the system without the addition of the composition.

Disclosed are methods of inhibiting TR4 transcription activity comprising administering a composition, wherein the composition prevents TR4 transcription activity, wherein the composition is defined as a composition capable of being identified by administering the composition to a system, wherein the system supports TR4 transcription activity, assaying the effect of the composition on the amount of transcription activity in the system, and selecting a composition which causes a decrease in the amount of TR4 transcription activity present in the system relative to the system without the addition of the composition.

Also disclosed are methods of inhibiting TR4 transcription activity comprising administering a composition that binds TR4, wherein the composition is AR or fragment thereof, or a molecule that competitively competes with TR4 for AR binding.

Also disclosed are methods of inhibiting TR4 transcription activity comprising administering a composition that binds TR4, wherein the composition is ER or fragment thereof, or a molecule that competitively competes with TR4 for ER binding.

Disclosed are methods of making a composition capable of inhibiting TR4 transcription activity comprising admixing a compound with a pharmaceutically acceptable carrier, wherein the compound is identified by administering the compound to a system, wherein the system supports TR4 transcription activity, assaying the effect of the compound on the amount of TR4 transcription activity in the system, and selecting a compound which causes a decrease in the amount of TR4 transcription activity in the system relative to the system without the addition of the compound.

Disclosed are methods of manufacturing an inhibitor to TR4 transcription activity comprising, a) administering a composition to a system, wherein the system supports TR4 transcription activity, b) assaying the effect of the composition on the amount of TR4 transcription activity in the system, c) selecting a composition which cause a decrease in the amount of TR4 transcription activity present in the system relative to the system with the addition of the composition, and d) synthesizing the composition.

Disclosed are methods of inhibiting TR4 transcription activity comprising administering a composition, wherein the composition prevents TR4 transcription activity, wherein the composition is defined as a composition capable of being identified by administering the composition to a system, wherein the system supports TR4 transcription activity, assaying the effect of the composition on the amount of transcription activity in the system, and selecting a composition which causes a decrease in the amount of TR4 transcription activity present in the system relative to the system without the addition of the composition.

Also disclosed are methods of inhibiting TR4 transcription activity comprising administering a composition that binds TR4, wherein the composition is ER, AR, or fragment thereof, or a molecule that competitively competes with AR or ER for TR4 binding.

Disclosed are methods of making a composition capable of inhibiting TR4 transcription activity comprising admixing a compound with a pharmaceutically acceptable carrier, wherein the compound is identified by administering the compound to a system, wherein the system supports TR4 transcription activity, assaying the effect of the compound on the amount of TR4 transcription activity in the system, and selecting a compound which causes a decrease in the amount of TR4 transcription activity in the system relative to the system without the addition of the compound.

Disclosed are methods of manufacturing an inhibitor to TR4 transcription activity comprising, a) administering a composition to a system, wherein the system supports TR4 transcription activity, b) assaying the effect of the composition on the amount of TR4 transcription activity in the system, c) selecting a composition which cause a decrease in the amount of TR4 transcription activity present in the system relative to the system with the addition of the composition, and d) synthesizing the composition.

Also disclosed are methods comprising the step of admixing the composition with a pharmaceutical carrier.

Disclosed are cells that further comprise an inhibitor of a TR4 transcription activity.

Disclosed are cells that further comprising an inhibitor of a ER or AR transcription activity.

11. Methods of Inhibiting ER Transcription Activity

Disclosed are methods of inhibiting ER transcription activity comprising administering a composition, wherein the composition prevents ER transcription activity, wherein the composition is defined as a composition capable of being identified by administering the composition to a system, wherein the system supports ER transcription activity, assaying the effect of the composition on the amount of transcription activity in the system, and selecting a composition which causes a decrease in the amount of ER transcription activity present in the system relative to the system without the addition of the composition.

Also disclosed are methods of inhibiting ER transcription activity comprising administering a composition that binds ER, wherein the composition is TR4 or fragment thereof, or a molecule that competitively competes with TR4 for ER binding.

Disclosed are methods of making a composition capable of inhibiting ER transcription activity comprising admixing a compound with a pharmaceutically acceptable carrier, wherein the compound is identified by administering the compound to a system, wherein the system supports ER transcription activity, assaying the effect of the compound on the amount of ER transcription activity in the system, and selecting a compound which causes a decrease in the amount of ER transcription activity in the system relative to the system without the addition of the compound.

Disclosed are methods of manufacturing an inhibitor to ER transcription activity comprising, a) administering a composition to a system, wherein the system supports ER transcription activity, b) assaying the effect of the composition on the amount of ER transcription activity in the system, c) selecting a composition which cause a decrease in the amount of ER transcription activity present in the system relative to the system with the addition of the composition, and d) synthesizing the composition.

Also disclosed are methods comprising the step of admixing the composition with a pharmaceutical carrier.

Disclosed are cells that further comprising an inhibitor of a ER transcription activity.

12. Methods of Inhibiting TR2 Transcription Activity

Disclosed are methods of inhibiting TR2 transcription activity comprising administering a composition, wherein the composition prevents TR2 transcription activity, wherein the composition is defined as a composition capable of being identified by administering the composition to a system, wherein the system supports TR2 transcription activity, assaying the effect of the composition on the amount of transcription activity in the system, and selecting a composition which causes a decrease in the amount of TR2 transcription activity present in the system relative to the system without the addition of the composition.

Also disclosed are methods of inhibiting ER transcription activity comprising administering a composition that binds ER, wherein the composition is TR2 or fragment thereof, or a molecule that competitively competes with TR2 for ER binding.

Disclosed are methods of making a composition capable of inhibiting TR2 transcription activity comprising admixing a compound with a pharmaceutically acceptable carrier, wherein the compound is identified by administering the compound to a system, wherein the system supports TR2 transcription activity, assaying the effect of the compound on the amount of TR2 transcription activity in the system, and selecting a compound which causes a decrease in the amount of TR2 transcription activity in the system relative to the system without the addition of the compound.

Disclosed are methods of manufacturing an inhibitor to TR2 transcription activity comprising, a) administering a composition to a system, wherein the system supports TR2 transcription activity, b) assaying the effect of the composition on the amount of TR2 transcription activity in the system, c) selecting a composition which cause a decrease in the amount of TR2 transcription activity present in the system relative to the system with the addition of the composition, and d) synthesizing the composition.

Also disclosed are methods comprising the step of admixing the composition with a pharmaceutical carrier.

Disclosed are cells that further comprising an inhibitor of a TR2 transcription activity.

Also disclosed are methods of identifying inhibitors of TR2 transcription activity comprising, a) administering a composition to a system, wherein the system supports TR2 transcription activity, b) assaying the effect of the composition on the amount of TR2 transcription activity in the system, and c) selecting a composition which causes a decrease in the amount of TR2 transcription activity present in the system relative to the system without the addition of the composition.

Disclosed are methods of inhibiting TR2 transcription activity comprising administering a composition, wherein the composition prevents TR2 transcription activity, wherein the composition is defined as a composition capable of being identified by administering the composition to a system, wherein the system supports TR2 transcription activity, assaying the effect of the composition on the amount of transcription activity in the system, and selecting a composition which causes a decrease in the amount of TR2 transcription activity present in the system relative to the system without the addition of the composition.

Also disclosed are methods of inhibiting TR2 transcription activity comprising administering a composition that binds TR2, wherein the composition is AR or fragment thereof, or a molecule that competitively competes with TR2 for AR binding.

Disclosed are methods of making a composition capable of inhibiting TR2 transcription activity comprising admixing a compound with a pharmaceutically acceptable carrier, wherein the compound is identified by administering the compound to a system, wherein the system supports TR2 transcription activity, assaying the effect of the compound on the amount of TR2 transcription activity in the system, and selecting a compound which causes a decrease in the amount of TR2 transcription activity in the system relative to the system without the addition of the compound.

Disclosed are methods of manufacturing an inhibitor to TR2 transcription activity comprising, a) administering a composition to a system, wherein the system supports TR2 transcription activity, b) assaying the effect of the composition on the amount of TR2 transcription activity in the system, c) selecting a composition which cause a decrease in the amount of TR2 transcription activity present in the system relative to the system with the addition of the composition, and d) synthesizing the composition.

Disclosed are cells that further comprising an inhibitor of a TR2 transcription activity.

Disclosed are methods for screening a compound for use in treatment of androgen related diseases comprising the steps of testing the compound to determine the effect of the compound on nuclear receptor mediated transcriptional activity, the activity being mediated by a nuclear receptor selected from the group consisting of the TR2 orphan receptor, the TR4 orphan receptor, and the RXR receptor, and observing the effect of such compound on the level of androgen receptor initiated transcription in the test.

Disclosed are methods for screening a compound for use in treatment of estrogen related diseases comprising the steps of testing the compound to determine the effect of the compound on nuclear receptor mediated transcriptional activity, the activity being mediated by a nuclear receptor selected from the group consisting of the TR2 orphan receptor, the TR4 orphan receptor, and the RXR receptor, and observing the effect of such compound on the level of estrogen receptor initiated transcription in the test.

Disclosed are methods for modulating the sensitivity of a cell to a sex hormone comprising the step of stimulating in the cell the abundance of a nuclear receptor selected from the groups consisting of the TR2 orphan receptor, the TR4 orphan receptor and the RXR receptor.

Also disclosed are methods for modulating androgen receptor-mediated transactivation activity in a cell, comprising the step of: treating the cell with a compound that can modulate TR2 orphan receptor level or TR4 orphan receptor level in the cell.

Disclosed are methods for down regulating androgen receptor-mediated transactivation activity in a cell, comprising the step of introducing TR2 orphan receptor ligand binding domain or TR4 orphan receptor ligand binding domain into the cell.

Also disclosed are methods for modulating estrogen receptor-mediated transactivation activity in a cell, comprising the step of: treating the cell with a compound that can change TR2 orphan receptor level or the TR4 orphan receptor level in the cell.

Disclosed are methods, wherein the agent is TR2 receptor, and/or wherein the compound is the TR2 orphan receptor ligand binding domain.

Disclosed are methods for down regulating TR2 orphan receptor-mediated transactivation activity in a cell, comprising the step of: introducing estrogen receptor ligand binding domain into the cell.

Disclosed are methods for screening a compound for treating androgen receptor-related diseases, comprising the step of: exposing cells to the compound; and determining the effect of the compound on TR2 or TR4 orphan receptor signaling pathway in the cells.

Disclosed are methods, wherein the effect on TR4 orphan receptor signaling pathway is measured by TR4 orphan receptor level, and/or wherein the effect on TR4 orphan receptor signaling pathway is measured by TR4 orphan receptor-mediated transactivation activity.

Disclosed are methods for screening a compound for treating estrogen receptor-related diseases, comprising the step of: exposing cells to the compound; and determining the effect of the compound on TR2 orphan receptor signaling pathway in those cells.

Disclosed methods, wherein the effect on TR2 orphan receptor signaling pathway is measured by TR2 orphan receptor levels, and/or wherein the effect on TR2 orphan receptor signaling pathway is measured by TR2 orphan receptor-mediated transactivation activity.

Disclosed are compositions that interact with ER, wherein the interaction occurs within the region defined by amino acids 312 to 340 of ER and methods of inhibiting transcription activity of ER comprising administering a composition, wherein the composition interacts within amino acids 312 to 340 of ER.

C. Compositions

It has been known for two decades that steroid/thyroid hormones function through the action of specific receptor proteins (Mangelsdorf, D. J. et al. (1995) Cell 83:835-839; Tingley, D. W. (1996) J. NIH Res. 8:81-88). Steroid/thyroid hormone receptors comprise a huge family of transcriptional factors that regulate complex gene networks in a wide variety of biological processes, such as growth, development, and differentiation (Evans, R. M. (1988) Science 240, 889-895), and include more than 150 proteins so far identified. Members of this superfamily include receptors for steroid hormones, thyroid hormones, vitamin A and D derivatives, as well as a large group of orphan receptors whose cognate ligands remain to be identified. Upon binding to their respective hormonal ligands, the steroid hormone receptors can undergo an activation or transformation step (O'Malley, B. W. et al. (1992) Biol. Reprod. 46, 163-1675; Truss, M. et al. (1993) Endocrine Rev. 14, 459-479). Regardless of whether transcriptional activity is controlled by the binding of a ligand or not, each of these proteins must be capable of binding to a specific DNA sequence that identifies particular genes as targets for regulation. The interactions between the protein and the DNA are mediated by highly conserved DNA binding domains (DBD) of each protein, the presence of the DBD defining the nuclear receptor superfamily. Interactions between proteins, necessary for the formation of homodimers and/or heterodimers, are mediated by an extensive carboxyl terminal dimerization interface that is contained within the ligand binding domain (LBD) in each of these proteins. Mangelsdorf, et al., Cell 83, 851-857 (1995). The members of this superfamily are often classified by the method of dimerization as well as the agent which activates the receptor. Homodimeric receptors in this family include the receptors for androgen (AR), glucocorticoid, estrogen (ER), and mineralocorticoid and a large diverse subfamily of non-steroid receptors including receptors for thyroid hormone, retinoids and vitamin D, as well as many orphan receptors, for which the majority will heterodimerize with retinoid X receptor (RXR). These RXR heterodimers function as dynamic transcription factors in which one subunit influences the capacity of the other subunit to interact with the ligand and with other co-factors. Forman, et al., Cell 81, 541-550 (1995); Kurokawa, et al., Nature 371, 528-531 (1994); Schulman et al., Genes Dev. 11, 299-308 (1997); Zamir, et al., Genes Dev. 11, 835-846 (1997); Wiebel, & Gustafsson, Mol. Cell. Biol. 17, 3977-3986 (1997). Another common heterodimer partner, known as the short heterodimer partner, like RXR, can interact with various nuclear receptors and acts as a negative regulator for the nuclear receptor signaling pathway. Seol, et al., Science 272, 1336-1339 (1996). The hormone-receptor complex, serving as a trans-regulator, can specifically bind to a cis-acting DNA sequence, known as a hormone response element (HRE), and thereafter regulate the transcription of target genes (Mangelsdorf, D. J. et al. (1995) Cell 83, 841-850). The orphan receptors belong to the nuclear receptor superfamily which mediates extracellular hormonal signals to transcriptional response. The roles of orphan receptors have been linked to development, homeostasis, and diseases. (Mangelsdorf, D. et al. (1995) Cell 83(6) 841-50-3; Enmark, E., et al. (1996) Mol. Endocrinol. 10(11) 1293-307; Giguere, V. (1999) Endocr. Rev. 20(5), 689-725). Orphan receptor is used to group receptor-like proteins that have no known ligand or function (Mangelsdorf, D. J. et al. (1995) Cell 83, 841-850; O'Malley, B. W. et al. (1992) Mol. Endocrinol. 6, 1359-1361).

Many human diseases and clinical conditions are related to cellular functions mediated by nuclear receptors such as, among others, AR, ER, TR4 and TR2. For example, AR and ER are involved in prostate cancer and breast cancer, respectively. TR4 orphan receptor and TR2 orphan receptor can be involved in neuro-brain function loss through their regulation of the ciliary neurotrophic factor receptor gene. Young, et al., J Biol. Chem. 272, 3109-3116 (1997); Young, et al., J Biol. Chem. 273, 20877-20885 (1998). Other evidence that TR4 and TR2 can be involved in a variety of human clinical conditions come from the fact that they suppress retinoic acid (RA)-induced trans activation, Lin, et al., J Biol. Chem. 270,30121-30128 (1995), Lee, et al., J Biol. Chem. 273, 13437-13443 (1998), recognize a DNA promoter in Simian virus 40, Lee, & Chang, J Biol. Chem. 2705,5434-5440 (1995), Lee, et al., J Biol. Chem. 270,30129-301.3 (1995), modulate thyroid hormone and vitamin D signal cascades, Lee, et al., J Biol. Chem. 272,12215-12220 (1997), Lee, et al., J Biol. Chem. 274, 13437-13443 (1998), and exert negative activities on the erythropoietin gene expression. Lee, & Chang, J Biol. Chem. 271, 10405-10412 (1996). TR4 is also observed to enhance the expression of human hepatitis B virus. Breidbart, et al., Pediatric Res. 34,300-302 (1993). TR2 is observed in most of the developing neural structures, Young, et al., J Biol. Chem. 273,20877-20885 (1998), suggesting that TR2 plays an important role in the development process of nervous system. In addition, the TR2 mRNA levels are abundantly distributed in many tissues of male rat reproductive organs and highly expressed in mouse embryos beginning at embryonic day 9 and in adult testis, indicating that the TR2 has a deep involvement in early reproductive functions. Lin, et al., J: Biol. Chem. 270,30121-30128 (1995).

Modulating nuclear receptor transactivating activity has been proved successful in treating diseases that are related to such nuclear transactivating activity. For example, certain types of breast cancer can be controlled by blocking the estrogen receptor transactivation using the antiestrogen tamoxifen.

1. Molecules That Inhibit TR2/TR4 Interactions

a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of AR, ER, TR2, or TR4 or the genomic DNA of AR, ER, TR2, or TR4 or they can interact with the polypeptide AR, ER, TR2, or TR4. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of TR2, TR4, AR, or ER, or fragments thereof, aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target molecule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162

b) Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with TR2, TR4, AR, or ER, or fragments thereof such that TR2, TR4, AR, or ER, are inhibited from performing transactivation activity. Antibody also includes, chimeric antibodies and hybrid antibodies, with dual or multiple antigen or epitope specificities, and fragments, such as F(ab′)2, Fab′, Fab and the like, including hybrid fragments, as well as conjugates of antibody fragments and antigen binding proteins (single chain antibodies) as described, for example, in U.S. Pat. No. 4,704,692, the contents of which are hereby incorporated by reference. Antibodies that bind the disclosed regions of TR2, TR4, AR, or ER, or fragments thereof, such that TR2, TR4, AR, or ER, decrease their transactivation activity are also disclosed. The antibodies can be tested for their desired activity using the in vitro assays described herein, or by analogous methods, after which their in vivo therapeutic and/or prophylactic activities are tested according to known clinical testing methods. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “monoclonal antibody” as used herein refers to an antibody obtained from a substantially homogeneous population of antibodies, i.e., the individual antibodies within the population are identical except for possible naturally occurring mutations that can be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes can be immunized in vitro, e.g., using the binding domains of the compositions described, herein, such as the ligand binding domain, described herein.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

(2) Human antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5, 939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti TSG101 antibodies and antibody fragments of the invention can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

c) Compositions Identified by Screening with Disclosed Compositions/Combinatorial Chemistry

(1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein, such as TR2, TR4, AR, or ER, or fragments thereof, can be used as targets for the combinatorial approaches. Also disclosed are the compositions that are identified through combinatorial techniques or screening techniques in which the compositions disclosed in herein, such as TR2, TR4, AR, or ER, or fragments thereof, or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule's function. The molecules identified and isolated when using the disclosed compositions, such as, TR2, TR4, AR, or ER, or fragments thereof, are also disclosed. Thus, the products produced using the combinatorial or screening approaches that involve the disclosed compositions, such as, TR2, TR4, AR, or ER, or fragments thereof, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptidyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice, for example a portion of TR2, TR4, AR, or ER, is attached to a DNA binding domain of a transcriptional activation protein, such as Gal 4. By performing the Two-hybrid technique on this type of system, molecules that bind the portion of TR2, TR4, AR, or ER, can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. No. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. No. 5,698,685 and 5,506,337), sulfamides (U.S. Pat. No. 5,618,825), and benzodiazepines (U.S. Pat. No. 5,288,514).

Screening molecules similar to TR2, TR4, AR, or ER, or fragments thereof for inhibition of TR2, TR4, AR, or ER, activity is a method of isolating desired compounds.

Molecules isolated which bind TR2, TR4, AR, or ER, or fragments thereof, are typically competitive inhibitors so that the heterodimerization properties, such as inhibition of TR2, TR4, AR, or ER, transactivation activity, possessed between TR2 and ER as well as TR4 and ER and TR4 and AR are present. Disclosed are molecules, such as fragments of TR2 and TR4 which bind to AR or ER competitively with TR2 and TR4.

In another embodiment the inhibitors are non-competitive inhibitors, which, for example, cause allosteric rearrangements which prevent TR2, TR4, AR, or ER, activity such as the heterodimers disclosed herein.

As used herein combinatorial methods and libraries included traditional screening methods and libraries as well as methods and libraries used in iterative processes.

(2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule's function. The molecules identified and isolated when using the disclosed compositions, such as, AR, ER, TR2, TR4, and/or fragments thereof, are also disclosed. Thus, the products produced using the molecular modeling approaches that involve the disclosed compositions, such as, AR, ER, TR2, TR4, and/or fragments thereof, are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

d) Methods of Identifying Inhibitors of AR-TR4 Interactions

Disclosed are methods of identifying an inhibitor of an interaction between AR and TR4, comprising incubating a library of molecules with AR forming a mixture, and identifying the molecules that disrupt the interaction between AR and TR4, wherein the interaction disrupted comprises an interaction between the AR and TR4 binding site.

Also disclosed are methods, wherein the step of isolating comprises incubating the mixture with a molecule comprising TR4.

Disclosed are methods of identifying an inhibitor of an interaction between AR and TR4 comprising incubating a library of molecules with TR4 forming a mixture, and identifying the molecules that disrupt the interaction between AR and TR4, wherein the interaction disrupted comprises an interaction between the AR and TR4 binding site.

Also disclosed are the methods, wherein the step of isolating comprises incubating the mixture with molecule comprising AR.

Also disclosed are compositions produced by any of the processes as disclosed herein, as well as compositions capable of being identified by the processes disclosed herein.

Disclosed are methods of manufacturing a composition for inhibiting the interaction between AR and TR4 comprising synthesizing the inhibitors as disclosed herein.

Also disclosed are methods that include mixing a pharmaceutical carrier with the inhibitor as disclosed herein, and produced by any of the disclosed methods.

Disclosed are methods of identifying inhibitors of AR and TR4 interaction comprising, a) administering a composition to a system, wherein the system supports AR and TR4 interaction, b) assaying the effect of the composition on the amount of AR-TR4 in the system, and c) selecting a composition which causes a decrease in the amount of AR-TR4 present in the system relative to the system without the addition of the composition.

Also disclosed are methods of identifying inhibitors of AR transcription activity comprising, a) administering a composition to a system, wherein the system supports AR transcription activity, b) assaying the effect of the composition on the amount of AR transcription activity in the system, and c) selecting a composition which causes a decrease in the amount of AR transcription activity present in the system relative to the system without the addition of the composition.

e) Methods of Identifying Inhibitors of ER-TR4 Interaction

Disclosed are methods of identifying an inhibitor of an interaction between ER and TR4, comprising incubating a library of molecules with ER forming a mixture, and identifying the molecules that disrupt the interaction between ER and TR4, wherein the interaction disrupted comprises an interaction between the ER and TR4 binding site.

Also disclosed are methods, wherein the step of isolating comprises incubating the mixture with molecule comprising TR4.

Disclosed are methods of identifying an inhibitor of an interaction between ER and TR4 comprising incubating a library of molecules with TR4 forming a mixture, and identifying the molecules that disrupt the interaction between ER and TR4, wherein the interaction disrupted, comprises an interaction between the ER and TR4 binding site.

Also disclosed are the methods, wherein the step of isolating comprises incubating the mixture with molecule comprising ER.

Also disclosed are compositions produced by any of the processes as disclosed herein, as well as compositions capable of being identified by the processes disclosed herein.

Disclosed are methods of manufacturing a composition for inhibiting the interaction between ER and TR4 comprising synthesizing the inhibitors as disclosed herein.

Also disclosed are methods that include mixing a pharmaceutical carrier with the inhibitor as disclosed herein, and produced by any of the disclosed methods.

Disclosed are methods of identifying inhibitors of ER and TR4 interaction comprising, a) administering a composition to a system, wherein the system supports ER and TR4 interaction, b) assaying the effect of the composition on the amount of ER-TR4 in the system, and c) selecting a composition which causes a decrease in the amount of ER-TR4 present in the system relative to the system without the addition of the composition.

Also disclosed are methods of identifying inhibitors of ER transcription activity comprising, a) administering a composition to a system, wherein the system supports ER transcription activity, b) assaying the effect of the composition on the amount of ER transcription activity in the system, and c) selecting a composition which causes a decrease in the amount of ER transcription activity present in the system relative to the system without the addition of the composition.

f) Methods of Identifying Inhibitors of T2-ER or AR Interactions

It is understtod that AR can be substituted for ER in method claims related to TR2 as well as TR4, as all molecules interact.

Disclosed are methods of identifying an inhibitor of an interaction between ER and TR2, comprising incubating a library of molecules with ER forming a mixture, and identifying the molecules that disrupt the interaction between ER and TR2, wherein the interaction disrupted comprises an interaction between the ER and TR2 binding site.

Also disclosed are methods, wherein the step of isolating comprises incubating the mixture with molecule comprising TR2.

Disclosed are methods of identifying an inhibitor of an interaction between ER and TR2 comprising incubating a library of molecules with TR2 forming a mixture, and identifying the molecules that disrupt the interaction between ER and TR2, wherein the interaction disrupted comprises an interaction between the ER and TR2 binding site.

Also disclosed are the methods, wherein the step of isolating comprises incubating the mixture with molecule comprising ER.

Also disclosed are compositions produced by any of the processes as disclosed herein, as well as compositions capable of being identified by the processes disclosed herein.

Disclosed are methods of manufacturing a composition for inhibiting the interaction between ER and TR2 comprising synthesizing the inhibitors as disclosed herein.

Also disclosed are methods that include mixing a pharmaceutical carrier with the inhibitor as disclosed herein, and produced by any of the disclosed methods.

Disclosed are methods of identifying inhibitors of ER and TR2 interaction comprising, a) administering a composition to a system, wherein the system supports ER and TR2 interaction, b) assaying the effect of the composition on the amount of ER-TR2 in the system, and c) selecting a composition which causes a decrease in the amount of ER-TR2 present in the system relative to the system without the addition of the composition.

Also disclosed are methods of identifying inhibitors of TR2 transcription activity comprising, a) administering a composition to a system, wherein the system supports TR2 transcription activity, b) assaying the effect of the composition on the amount of TR2 transcription activity in the system, and c) selecting a composition which causes a decrease in the amount of TR2 transcription activity present in the system relative to the system without the addition of the composition.

2. Aspects applicable to all compositions

a) Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example AR, ER, TR2, TR4, and/or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

(1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

(2) Sequences

There are a variety of sequences related to the genes of AR, ER, TR2, TR4, and/or fragments, which can be found at Genbank, at for example, http://www.pubmed.gov and these sequences and others are herein incorporated by reference in their entireties as well as for individual subsequences contained therein.

The disclosed sequences and variants can be founding Genbank. It is understood that the description related to this sequence is applicable to any sequence unless specifically indicated otherwise. Those of skill in the art understand how to resolve sequence discrepancies and differences and to adjust the compositions and methods relating to a particular sequence to other related sequences. Primers and/or probes can be designed for any sequence given the information disclosed herein and known in the art.

(3) Primers and probes

Disclosed are compositions including primers and probes, which are capable of interacting with the AR, ER, TR2, or TR4 nucleic acids as disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the AR, ER, TR2, TR4, and/or fragments thereof, nucleic acid or region of the AR, ER, TR2, TR4, and/or fragments thereof, nucleic acid or they hybridize with the complement of the AR, ER, TR2, TR4, and/or fragments thereof nucleic acid or complement of a region of the AR, ER, TR2, TR4, and/or fragments thereof nucleic acid.

d) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991) Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

(1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as nucleic acids encoding AR, ER, TR2, TR4, and/or fragments thereof into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes arc removed from the adenovirus genome.

(c) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(d) Large Payload Viral Vectors

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

(2) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

(3) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

(1) Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

(2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

f) Peptides

(1) Protein variants

As discussed herein there are numerous variants of the AR, ER, TR2, TR4, proteins and/or fragments thereof that are known and herein contemplated. In addition, to the known functional AR, ER, TR2, TR4, and/or fragments thereof species homologs there are derivatives of the AR, ER, TR2, TR4, proteins and/or fragments thereof, which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 16 sets forth a particular sequence of TR4 and SEQ ID NO:1 sets forth a particular sequence of a TR2 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence can not be written out herein, it is understood that each and every sequence is in fact disclosed and described herein through the disclosed protein sequence. For example, one of the many nucleic acid sequences that can encode the protein sequence set forth in SEQ ID NO:16 is set forth in SEQ ID NO:17. Another nucleic acid sequence that encodes the same protein sequence set forth in SEQ ID NO:16 is set forth in SEQ ID NO:18 In addition, for example, a disclosed conservative derivative of SEQ ID NO:16 is shown in SEQ ID NO:19, where the isoleucine (I) at position 8 is changed to a valine (V). It is understood that for this mutation all of the nucleic acid sequences that encode this particular derivative of the TR4 protein are also disclosed including for example SEQ ID NO:20 which sets forth a particular nucleic acid sequence that encodes the particular polypeptide set forth in SEQ ID NO:19. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

g) Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials can be in solution, suspension (for example, incorporated into microparticles, liposomes, or cells). These can be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

(1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Therapeutic Uses

Effective dosages and schedules for administering the compositions can be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody or other molecule, such as fragment of TR4, for forming or mimicking a TR4/AR or ER interaction, for example, the efficacy of the therapeutic antibody or fragment can be assessed in various ways well known to the skilled practitioner. For instance, one of ordinary skill in the art will understand that a composition, such as an antibody or fragment, disclosed herein is efficacious in forming or mimicking a TR4/AR or ER interaction in a subject by observing, for example, that the composition reduces the amount of AR or ER activity. The AR and Er activity can be measured using assays as disclosed herein. Any change in activity is disclosed, but a 5%, 10%, 15%, 20%, 25%, 30%, 35%, 40%, 45%, 50%, 55%, 60%, 65%, 70%, 75%, 80%, 90%, or a 95% reduction in AR or ER activity are also disclosed.

Other molecules that interact with TR2, TR4, AR, or ER to inhibit TR4/AR interactions, TR2/ER interactions, or TR4/ER interactions, which do not have a specific pharmacuetical function, but which can be used for tracking changes within cellular chromosomes or for the delivery of diagnositc tools for example can be delivered in ways similar to those described for the pharmaceutical products.

The disclosed compositions and methods can also be used for example as tools to isolate and test new drug candidates for a variety of TR2, TR4, AR, and ER related diseases.

h) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

i) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

3. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen el al., Bioconjug. Chem. 5:3-7 (1994).

2. Peptide synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions. For example, enzymatic ligation of cloned or synthetic peptide segments allow relatively short peptide fragments to be joined to produce larger peptide fragments, polypeptides or whole protein domains (Abrahmsen L. et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate including a human, ape, monkey, orangutan, or chimpanzee.

Also disclosed are animals produced by the process of adding to the animal any of the cells disclosed herein.

E. Methods of Using the Compositions

1. Methods of Using the Compositions as Research Tools

The compositions can be used for example as targets in combinatorial chemistry protocols or other screening protocols to isolate molecules that possess desired functional properties related to TR2 and TR4 and AR and ER interactions. For example, TR2 and TR4 as well as ER and AR and their interaction domains can be used in procedures that will allow the isolation of molecules or small molecules that mimic their binding properties. For example, disclosed herein TR2 and ER interact. Libraries of molecules can be screen for interaction with ER that mimics the TR2/ER interaction by incubating the potential ER binding molecules with ER and then isolating those that are specifically competed off with TR2. There are many variations to this general protocol.

The disclosed compositions can also be used diagnostic tools related to diseases such as ER or AR related diseases or TR2/TR4 related diseases.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Method of Treating Disease, Such as Cancer

The disclosed compositions can be used to treat any disease where uncontrolled cellular proliferation occurs such as cancers. Disclosed are methods for inhibiting cancers related to TR2 or TR4 related cancers, that are related to androgen receptor and estrogen receptor. By inhibiting the transactivation activity of TR2, TR4, AR, and ER, cancers caused by gene activation related to these transactivators can be reduced. TR2, TR4, AR, and ER and their variants and derivatives can be used to regulate the transcription activity of each in any combination. For example, both AR and ER could be used to regulate the activity of TR2, in combination.

Disclosed are methods for inhibiting cancers related to TR2 or TR4 related cancers for example, as they relate to, for example, to HPV related cancers and retinoid related cancers, for example. HPV related cancers include precancer conditions such as cervical and anal dysplasias, other dysplasias, severe dysplasias, hyperplasias, a typical hyperplasias, and neoplasias. Other related HPV cancers include genital and vaginal cancers, as well as oral cancers. For example CIN1-CIN3. Also disclosed are methods for inhibiting cancers that are related to p53 and Rb.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Mutual Suppression Between Sex Hormone Receptors and Other Nuclear Receptors

a) TR4-AR Negative Regulation

(1) Materials and Methods

(a) GST Dull-Down Assay

GST-TR4 fusion protein and GST control protein were purified as instructed by the manufacturer (Pharmacia). Five ml of in vitro translated 35S methionine-labeled proteins were used to perform a pull-down assay as described previously. Cacailles, et al., EMBO 14, 3741-3751 (1995).

(b) Immunocytofluorescence

DU145 cells were seeded on two-well Lab Tek Chamber slides (Nalge Nunc International) 18 h before transfection. One to two μg of DNA per 10⁵ cells was transfected by the FuGENETM 6 transfection reagent (Boehringer-Mannheim). Transfected cells were treated with 100 nM DHT. Immunostaining was performed by incubating with anti-AR polyclonal antibody (NH27), anti-TR4 monoclonal antibody (#15), and anti-ERa monoclonal antibody (C-314, Santa Cruz), followed by incubation with either fluorescein-conjugated goat anti-rabbit or anti-mouse antibodies (ICN Pharmaceuticals, Inc.). Yang, et al., Proc. Natl. Acad Sci., USA 94, 13075-13080 (1997). The slides were photographed under 100-fold magnification using confocal microscopy.

(c) Transient Transfection

Cells were routinely maintained in DMEM with 10% heat-inactivated fetal bovine serum. The cells were transfected using a modified calcium phosphate precipitation method, Mizokami, & Chang, J: Biol. Chem. 269,25655-25659 (1994), or SuperFect (Qiagen). To normalize the transfection efficiency, the (3-galactosidase expression ver and pRL-TK were co-transfected in CAT assay and in Dual-luciferase reporter assay system (Promega), respectively.

(d) Electrophoretic Mobility Shift Assay

EMSA was performed as described previously. Lee, et al., J: Biol. Chem. 272, 12215-12220 (1997). Briefly, the reaction was performed by incubating the 32P-end labeled DR1 probe with in vitro translated TR4 (1 ml) with or without an increasing amount of AR (1, 2, 4 ml). The EMSA incubation buffer is 10 mM HEPES, pH 7.9, 2% (v/v) glycerol, 100 mM KCl, 1 mM EDTA, 5 mM MgCl, and 1 mM DTT. For the antibody supershifted analysis, 1 ml of monoclonal anti-TR4 antibody (#15) was added to the reaction. DNA-protein complexes were resolved on a 5% native gel. The radioactive gel was analyzed by autoradiography.

(e) Northern Blotting: Analysis

Total RNA from the DHT-treated transfected LNCaP cells was prepared by the ultracentrifugation method as described. Lee, et al., J: Biol. Chem. 273, 13437-13443 (1998). The probe was obtained from exon 3 of PSA gene and labeled with a-32p dCTP.

(2) Results

(a) TR4 Interacts with AR both In Vitro and In Vivo.

Using a GAL4-TR4 fusion protein as bait, the yeast two-hybrid system was used to isolate several potential TR4 associated proteins. Sequence analysis confirmed that some of the candidates, such as AR and the TR2 orphan receptor, could physically interact with TR4. A GST-TR4 fusion protein pull-down assay was performed to further confirm the result. GST-TR4 fusion protein and GST control protein were purified as instructed by the manufacturer (Pharmacia). Five μl of in vitro translated 35S methionine-labeled AR, TR2, and RXRα were incubated with GST-TR4 or GST bound to glutathione-Sepharose beads in a pull down assay. TR4 was found to physically interact with AR and TR2 orphan receptor. In contrast, there was no interaction between TR4 and RXR, another member of the steroid receptor superfamily.

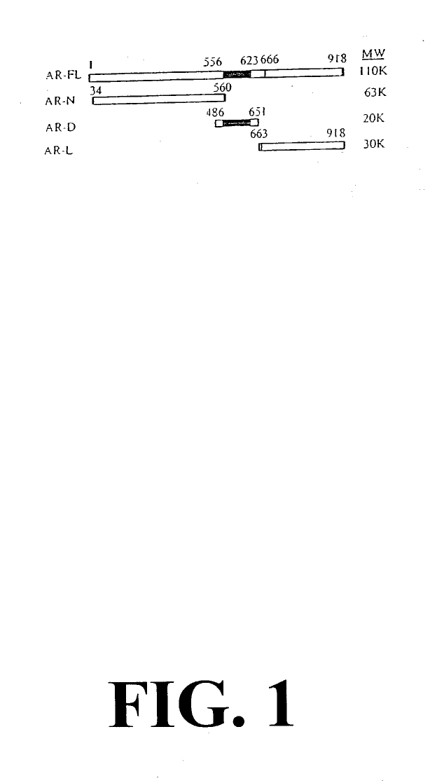

AR was further characterized due to its profound effects on many androgen-related diseases. To map more precisely the regions in AR that can interact with TR4, various AR deletion mutants, AR-N, AR-D, and AR-L were in vitro translated and incubated with GST-TR4 in a pull-down assay. FIG. 1 illustrates the deletions associated with each of those deletion mutants. The pull-down complex was loaded onto an 8% or 15% polyacrylamide gel and visualized by autoradiography. TR4 was found to interact with three in vitro-translated 35S methionine AR deletion constructs, the N-terminal of AR (AR-N), the DBD of AR (AR-D), and the LBD of AR (AR-L). These results agreed with previous reports that coregulators were able to interact with both N-terminal and C-terminal domains of steroid receptors. Gelman, et al., J. Biol. Chem. 274,7681-7688 (1999).

An immunocytofluorescence assay was then applied to determine the subcellular localization of the AR and TR4 in DU145 cells. DU145 cells were seeded on two-well Lab Tek Chamber slides (Nalge Nunc International) 18 hours before transfection. One to two μg of DNA per 105 cells was transfected either with AR (unliganded or liganded), TR4, or ER alone or in combination with the FuGENETM6 transfection reagent (Boehringer-Mannheim). 24 hours after transfection, cells were treated with 100 nM DHT or ethanol. Immunostaining was performed by incubation with the anti-AR polyclonal antibody, anti-TR4 monoclonal antibody, or anti-ERa monoclonal antibody, followed by incubation with either fluorescein-conjugated goat anti-rabbit or anti-mouse antibodies (ICN Pharmaceuticals, Inc.). It was found that unliganded AR was located mainly in the cytoplasm. The AR signal moved to the nucleus in the presence of its cognate ligand, DHT. These data agreed with a previous report shown in COS cells. Simental, et al., J: Biol. Chem. 266, 510-518 (1991). In contrast, TR4 was detected as a nuclear protein as was ER even in the absence of exogenous ligand.

When AR and TR4 were co-transfected into DU14S cells the majority of the AR signal could be detected together with TR4 signal in the nucleus, even in the absence of DHT. This data indicates that unliganded cytosolic AR moves into the nucleus once it is coexpressed with TR4 in DUI45 cells. In contrast, when AR and ER were co-transfected into DUI45 cells, the AR signal still remained mainly in the cytoplasm in a manner similar to that found when AR was transfected alone. The observation that unliganded AR can translocated into the nucleus in the presence of TR4 provides strong in vivo evidence that AR interacts specifically with TR4.

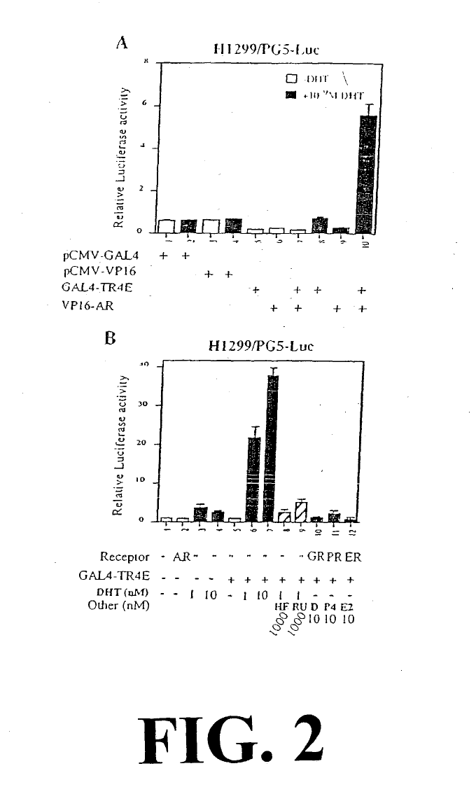

The ability of TR4 to interact with AR was further evaluated by the mammalian two-hybrid system assay. A near full-length human AiR (amino acids 33-918) was fused to the transcriptional activator VPL6 (VP16-AR) and then co-transfected with GAL4-DBD fused with TR4 LBD (GAL4-TR4E) and a GAL4-responsive luciferase reporter (PG5-Luc) in H1299 cells. More specifically, 3.5 μg of PG5-Luc, the luciferase reporter gene containing five copies of GAL-DBD binding sites, was cotransfected with two fusion proteins, GAL4-TR4E, and VPI6-AR. After 16-18 hours transfection, 1 nM DHT was added and ethanol was used in control groups. After 24 hours treatment, cells were harvested for Dual Luciferase Assay. The results, illustrated in FIG. 2A, revealed that either parental vector (pCMV-GAL4 or pCMV-VPI6), VPI6-AR, or GAL4-TR4E alone; showed a low background in the absence or presence of 1 nM DHT. Upon co-transfection of VPI6-AR and GAL4-TR4E, a significant induction was only observed when 1 nM DHT was added (see lane 10), thus indicating that DHT could promote the interaction between GAL4-TR4E and VPL6-AR.

We used a modified mammalian one-hybrid system to avoid the possibility that the DHT-dependent interaction between AR and TR4 is due to artificial conformational changes created by the VPI6-AR fusion protein in the mammalian two-hybrid system. A full-length AR(pSG5AR) was co-transfected with GAL4-TR4E and PG5-Luc reporter in H1299 cells (pSG5AR, pSG5GR, pSG5PR, disclosed in Yeh et al., Proc Natl. Acad. Sci., 93:5517-5521 (1996), which is herein incorporated by reference at least for material related to plasmids and assays, including sequence). More specifically, 3.5 μg of PG5-Luc and 3 μg of GAL4-TR4E were co-transfected in the presence of 1 μg of pSG5AR, pSG5GR, pSG5PR, or pSG5ER (pSG5ER found in Thombum et al., Nucleic Acids Res., 16:10469-76,(1988) which is herein incorporated by reference at least for material related to plasmids and assays, including sequence). Cells were treated as indicated in FIG. 2B.

Transfection was performed by a modified calcium phosphate precipitation method. The pRL-TK plasmid was co-transfected for normalization of transfection efficiency. As shown in FIG. 28, transfection of pSG5AR alone showed only marginal DHT-dependent transcriptional induction (lanes 2, 3, and 4), whereas, co-transfection of pSG5AR and GAL4-TR4E showed a significant (20-40 fold) induction in the presence of 1-10 nM DHT (lanes 6 and 7). In contrast, no induction was observed when AR was replaced with other activated steroid receptors, such as glucocorticoid receptor (GR), progesterone receptor (PR), or estrogen receptor (ER) (lanes 10, 11, and 12). Moreover, addition of 1 μM of anti androgens, such as hydroxyflutamide or RU58841, could abolish the DHT-enhanced interaction between AR and TR4 (lanes 8 and 9).

The difference between DHT-dependent interaction detected in the mammalian one- or two-hybrid systems and DHT-independent interaction detected in the GST pull-down and the immunocytofluorescence assays, could be due to the involvement of AF-1 ligand-independent interaction in the GST pull-down and the immunocytofluorescence assays, which used the full length of TR4 containing AF-1, vs. AF-2 ligand-dependent interaction in the mammalian one- or two-hybrid system, which used only TR4 ligand binding domain containing AF-2. Taken together, data from the GST pull down assay, the immunocytofluorescence assay, and the experiments done with the mammalian one- and two-hybrid system provide strong evidence that AR and TR4 can interact in the absence or presence of DHT.

(b) AR Represses TR4-Mediated Transactivation.

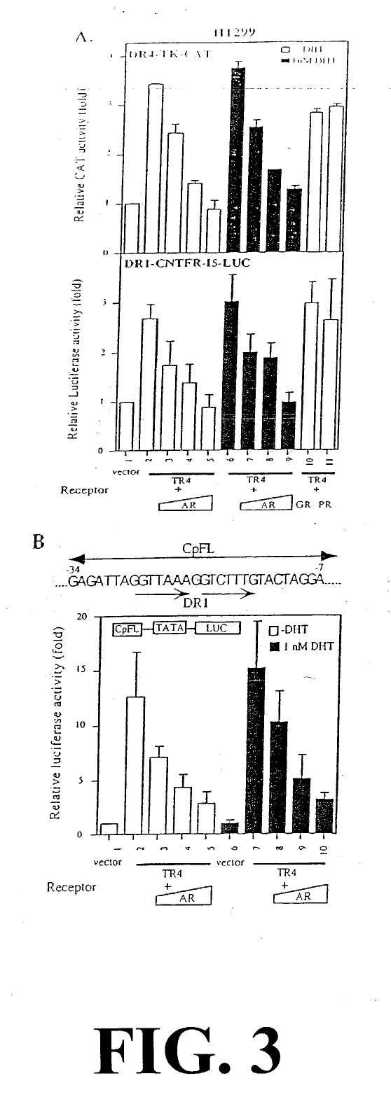

The potential effects on transactivation by the AR-TR4 heterodimer formation was then tested through use of a reporter assay: full-length AR and TR4 in eukaryotic expression vectors (pSG5AR and pCMXTR4) were co-transfected with a CAT reporter containing a TR4-response element (DR4-TK-CAT), Lee et al., J: Biol. Chem. 272, 12215-12220 (1997), in HI 299 cells. More specifically, 500 ng of reporter plasmids were co-transfected(DR4-CAT and CNTFR-15-LUC) with 200 ng of pCMX-TR4 and increasing amounts of pCMV-AR (200, 600, and 1,200 ng), pSG5GR (1,200 ng), or pSG5 PR (1,200 ng) using the SuperFect transfection kit (Qiagen) (PCMV AR Mowszowicz et al., (1993) Molecular Endocrinology 7:861:869). As shown in FIG. 3A, the CAT activity induced by pCMXTR4 could be repressed significantly, in a dose-dependent manner by co-transfection of pSG5AR in the absence or presence of DHT. This repression of TR4 transactivation is AR specific, as other activated steroid receptors, such as GR or PR, have no suppressive effects (FIG. 3A, lanes 9-10). Similar results were obtained when the DR4-TK-CAT reporter was replaced with DR1-CNTFR-15-LUC, another TR4 response element (FIG. 3A). Young, et al., J: Biol. Chem. 272,3109-3116 (1997).

Another TR4 potential target gene, which is located in the hepatitis B virus (HBV) enhancer II region (−34 to −7) containing a classic DR1 motif. Breidbart, et al., Pediatric Res. 34,300-302 (1993), was investigated. The reporter plasmid CpFL(4)-LUC, which contains the HBV core promoter (Cp) sequence located between −34 and −7 (nucleotide coordinates 1751 and 1778 derived from the Genbank database) was shown above in FIG. 3B. The arrows indicate the DRI motif in HBV core promoter. HepG2 cells were co-transfected with 1.5 μg CpFL(4)-LUC reporter and 0.5 μg pCMX-TR4, with increasing amounts of pCMV-AR (0.5, 2.5, and 5 μg) by modified calcium phosphate precipitation method. The relative reporter gene activities were compared to the CAT activities (or luciferase activities) with vector alone. To normalize the transfection efficiency, the β-galactosidase expression vector and pRL-TK were co-transfected in the CAT assay and in the Dual-luciferase reporter assay system (Promega), respectively. As shown in FIG. 3B, TR4 can induce CpFL(4)-LUC activity, which is significantly decreased by co-transfection of AR with TR4 in a dose-dependent manner. This finding suggested that AR could regulate HBV gene expression through protein-protein interaction.

(c) AR Prevents TR4from Binding to Its Target DNA.

EMSA using 32P labeled DR1-TR4RE as a probe were applied to further dissect the mechanism of how AR repressed the TR4-mediated transactivation. One μl of in vitro translated TR4 protein was incubated with increasing amounts of in vitro translated AR (1 μl, 2 μl, and 4 μl) in EMSA reaction buffer (10 mM HEPES pH 7.9, 2% (v/v) glycerol, 100 mM KCl, 1 mM EDTA, 5 mM MgCl2, and 1 mM DTT) for 15 min. 32P-end labeled DR1 was added into the protein mixture and incubated for 15 min before loading. For the antibody supershift assay, 1 μl of monoclonal anti-TR4 antibody was added to the reaction and applied to a 5% native polyacrylamide gel. The radioactive gel was analyzed by autoradiography. The specific TR4-DR1 band was decreased with the addition of increasing amounts of AR. Furthermore, the intensity of the supershifted band formed by the adding an anti-TR4 monoclonal antibody

(McAb) to the TR4-DR1 complex, was also decreased with the addition of increasing amounts of AR. Together, these results suggested that AR might be able to repress TR4-mediated transactivation by preventing TR4 from binding to its target DNA. As there is no extra supershifted band formed upon adding AR to TR4-DR1 complex, the data can also rule out the possibility of the formation of a transcriptional inactivated TR4-AR-DR1 complex.

(d) TR4 Represses AR Target Gene Activation Both In Vitro and Vivo

Like TR4, AR itself acts as a transcription factor to activate many androgen target genes. 500 ng of MMTV-Luc (FIG. 4A), or PSA-Luc (FIG. 4B), were co-transfected with 40 ng pCMV-AR (lane 2-3) with increasing amounts of pCMX-TR4 (as indicated in FIG. 4). After 24 hours transfection, cells were treated with 10 nM of DHT. After 16-18 hours incubation, cells were harvested for Dual-rnciferase reporter assay. As expected, in COS, H1299, and CHO cells, AR activated MMTV luciferase activity in a DHT-dependent manner (FIG. 4A, lanes 2 and 3), which could then be repressed by the addition of TR4 (lanes 6 to 8). TR4 by itself has no effect on MMTV ruciferase activity in the absence or presence of 10 nM DHT (lanes 4 and 5). Similar suppression effects also occurred when the MMTV-ruciferase reporter was replaced with the PSA-Iuciferase reporter, another AR target gene (FIG. 4B).

To rule out the potential artificial effects linked to transfected reporter assays, the expression of endogenous prostate specific antigen (PSA, an androgen target that is widely used as a marker for prostate cancer progression) in LNCaP cells was measured by Northern blot analysis. Total RNA (25 μg) from LNCaP cells, which were transfected with either pCMX-TR4 or pCMX vector using SuperFect (Qiagen), was applied into a formamide RNA gel, then transferred onto a Nylon membrane, and then hybridized with a 32P-PSA gene fragment from the exon 3. P-actin was used as an internal control. As shown in FIG. 4C, the expression of PSA transcript was induced about 2.5 fold after 24 hours of DHT treatment (lane 3 vs. 4). Addition of TR4 can clearly repress the expression of endogenous PSA transcript in either the absence (lane 1 vs. 3) or presence of 10 nM DHT (lane 2 vs. 4). The level of secreted PSA protein in the medium measured by ELISA, also confirmed this conclusion. This in vivo TR4-mediated suppressive effect strongly supports the above reporter assays.

(e) TR4 Represses AR-Mediated Transactivation Specifically.

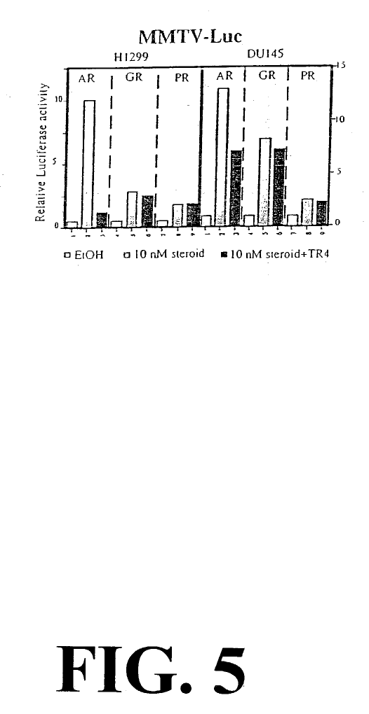

As GR and PR can also induce MMTV—luciferase reporter, Beato, M., Ce//56, 335-344 (1989), determining if TR4 could also repress OR- or PR-mediated transactivation was analyzed. Three μg of MMTV-Luc was co-transfected with 4 μg of pCMX-TR4 in the presence of 1 μg of pSG5AR, pSG5GR, or pSG5PR by modified calcium-phosphate method. After 24 hours transfection, the cells were treated with 10 nM of synthetic steroids (DHT, dexamethasone, and progesterone). Dual-luciferase reporter assays were performed. PRL-TK was used to normalize the transfection efficiency. As shown in FIG. 5, while AR, GR, and PR could induce MMTV-luciferase activity in the presence of their respective ligands in H1299 cells, co-transfection of TR4 could only repress AR-mediated transactivation. Similar results were observed when the same experiments of AR-mediated transactivation were repeated in DU145 cells.

Previous reports suggested that RXR could function as a coactivator through heterodimer formation with the receptors for Vitamin D (VDR), thyroid hormone (TR), and peroxisome proliferator (PPAR). Yu et al., Cell 67, 1251-1266 (1991); Kliewer et al., Nature 355, 446-449 (1992); Kliewer, et al., Nature 358,771-774 (1992); Zhang, et al., Nature 355, 441-446 (1992). The reverse repression effects of VDR, TR, and PPAR on RXR target genes, however, remain unknown. These bi-directional repression effects through the AR and TR4 heterodimerization, are a mechanism pathway in the steroid receptor superfamily signaling pathway. The physiological significance of the AR-TR 4 heterodimer is further supported by the similar expression pattern of both receptors in many tissues, such as the testis, hypothalamus, and prostate. Chang, et al., Proc. Natl. A cad. Sci USA. 91, 6040-6044 (1994); Lee, et al., J Biol. Chem. 274, 16198-16205 (1999); Chang, et al., Gene Expression 5, 97-126 (1995).

The role of AR in the modulation of androgen target genes can be expanded. In addition to activation of classic androgen target genes containing androgen response elements (GGA/TACAnnnTGTTCT), AR can also signal through heterodimerization with TR4, resulting in the repression of various TR4 target genes, which contain a consensus response element (AGGTCA) in a DR orientation (AGGTCA(n)ₓAGGTCA, x=0-6). Data from gel shift assays showed that the binding preference of TR4 for the natural TR4RE identified in various target genes, was in the order of DR1 (CRBI1-TR4RE)>DR2 (SV 40-TR4RE)>DR4 (TRE-TR4RE)>DR5 (RAREβ-TR4RE)>DR3 (VDRE-TR4RE), with the IC50 varying widely from 0.023 ng to 2.0 ng. Lee, et al., J: Biol. Chem. 273, 13437-13443 (1998); Lee, et al., J: Biol. Chem. 272, 12215-12220 (1997); Lee et al., J Biol. Chem. 274, 16198-16205 (1999); Lee et al. J Biol. Chem. 270,30129-30133 (1995). Demonstrated herein the classic androgen-signaling pathway (AARARE) can be influenced by TR4. This represents a mechanism to distinguish between receptors (AR, GR, and PR) that share the same hormone response elements (found in MMTV or other target genes), and provides a target through which to block the androgen action.

b) ER-TR2 Interactions

(1) Materials and Methods

(a) Chemicals and Plasmids

[Young, W. et al. (1997) J. Biol. Chem. 272, 3109-3116] Chloramphenical was obtained from Amersham Corp (Arlington Heights, Ill. 60005). Acetyl coenzyme A was purchased from Pharmacia Biotech Inc (Piscataway, N.J. 08854). 17β-estradiol (E2) was purchased from Sigma Chemical Co (St. Louis, Mo. 63178). The in vitro transcription/translation (TNT) coupled reticulocyte lysate system was purchased from Promega (Madison, Wis. 53711).

Human full-length ER was inserted into the EcoRI site of pSG5 to produce pSG5-ER. The pCMV-TR2, pCMX-VPI6-TR2, and pGEX-3x-TR2 were constructed by inserting full-length TR2 fragments to individual vectors. The GAL4-ER, was amino acids (aa) 282-595 of ER. Constructs corresponding to GST-ER fragments were made using the vector pGEX series (Pharmacia, Piscataway, N.J. 08854). Inserts of the ER fragments were released from the pSG5-ER and generated by the following strategies: GST-ER-N-terminal (GST-ER-N), the BamHI-MscI fragment of ER into the pGEX-3xSmaI-RcoRI site; GST-ER-DNA Binding Domain (GST-ER-DBD), the HindIII-PstI fragment into the pGEX-2T SmaI site; GST-ER-Ligand Binding Domain (GST-ER-LBD), the PstI-EcoRI fragment into the pGEX-2T SmaI-EcoRI site; and GST-ER-F Domain (GST-ER-F) the HhaI-EcoRI fragment into the pGEX-3xSmal site. All plasmids were verified by restriction enzyme analysis and DNA sequencing.

(b) Cell Culture

H1299 human lung cancer cells and PC-3 human prostate cancer cells were maintained in DMEM containing 5% fetal calf serum (FCS), 100 U/mL penicillin and 100 μg/ml streptomycin sulfate at 5% CO₂ at 37° C. T47D human breast cancer cells were maintained in RPMI 1640 medium containing 5% FCS, 100 U/ml penicillin and 100 μg/ml streptomycin sulfate at 5% CO₂ at 37° C.

(c) Transfections and Reporter Gene Expression Assays

Transfections and chloramphenicol acetyltransferase (CAT) assays were performed as described previously. Yeh, & Chang, Proc. Natl. Acad. Sci. 93, 5517-5521 (1996). Briefly, 4×105 cells were plated in 60-mm dishes and cultured for 24 h, and then the medium was changed to phenol red free DMEM with 5% charcoal-stripped FCS 2 h before transfection. The cells were co-transfected with TR2 and/or ER expression plasmids with 2 μg of TR2-TK-CAT reporter or 1 μg of ERE-CAT reporter gene plasmid, as indicated in the Figures, by using the calcium phosphate precipitation method. A β-gal actosidase expression plasmid, pCMV-β-gal, was transfected in all transfections as an internal control for normalizing transfection efficiency. The total amount of DNA was adjusted up to 10.5 μg with parent vectors, pSG5 or pCMV in each transcriptional activity assay. After 24 h transfection, the medium was changed again and the cells were treated with 10-8 M E2 for another 24 h. The cells were then harvested and whole cell extracts were used for CAT assay. The CAT activity was quantitated by Phosphorlmager (Molecular Dynamics). Data are presented as means±S.D. of at least three independent experiments.

(d) GST Pull-Down Assay

Fusion proteins of GST-TR2, GST-ER segments and GST were obtained by transforming expressing plasmids into BL21(DE3) pLysS competent bacteria followed with 2-hour IPTG induction. GST-proteins were then purified by mixing Glutathione-Sepharose™ 4B (Pharmacia) into bacteria lysates on a rotating disk at 4° C. for 40 mm followed by washing with 1 mL NENT buffer (20 mM Tris-HCL (pH 8.0), 100 mM NaCl, 1 mM EDTA, 6 mM MgCl2, 1 mM Dithiothretiol, 8% Glycerol, 1 mM PMSF and 0.5% (v/v) NP-40). The ER, AR, RXRA., and TR2 proteins labeled with [35S] were generated in vitro by using the TNT reticulocyte lysate system (Promega). For the in vitro interaction, the glutathione-Sepharose bound GST-proteins were resuspended with 100 μl of interaction buffer (20 mM HEPES pH 7.9, 150 mM KCl, 5 mM MgCl2. 0.5 mM EDTA, 0.5 mM Dithiothretiol, 0.1% (v/v) NP-40, 0.1% (w/v) BSA and 1 mM PMSF) and mixed with 5 μl of rotating disk at 4° C. for 3 h. After extensive washes with NENT buffer, the bound proteins were separated on an SDS/8% PAGE and visualized by using autoradiography.

(e) Mammalian Two-Hybrid Assay

Transfections were performed using the calcium-phosphate precipitation method described above. H1299 cells or PC-3 cells were transiently co-transfected with 3 μg of a GAL4-ER expression plasmid. 3 μg of a VPI6-TR2 expression plasmid and 3 μg pG5-CAT reporter plasmid. CAT assays were performed as described above. 1 μg of a (β-galactosidase expression plasmid, pCMV-βgal, was used as an internal control. The total amount of DNA was adjusted to 10.5 μg with parent vectors in all transcriptional activity assays.

(f) Electrophoretic Mobility Shift Assay EMSA)

EMSA was carried out as described previously with some modification. Lee et al., J: Biol. Chem. 274. 13437-13443 (1998). 0.1 μg of Double-stranded oligonucleotide ERE primers were end-labeled with 5 μl of [γ-32P]ATP (DuPont NEN) by using T4 polynucleotide kinase. In vitro translated proteins. 1 μl of ER protein and 2 μl of TR2, were incubated with the 0.1 ng of [32P]-labeled ERE probe (4×108 dpm/μg) in 20 μl of EMSA binding buffer (50 mM HEPES pH 7.9, 500 mM KCl, 5 mM Dithiothretiol, 2.5 mM EDTA, 12.5 mM MgCl2. 30% glycerol. 10% Ficoll) on ice for 30 min. For the competition reactions, 100 ng unlabeled ERE oligonucleotides were mixed with the labeled probe prior to addition to the reactions. For the antibody supershift assays. 1 μl of the monoclonal anti-ERa antibody (C-314, Santa Cruz) was added into the reactions for additional 30 min. The protein-DNA complex was analyzed on a 5% polyacrylamide native gel containing 2.5% glycerol in 0.5×TBE buffer (45 mM Tris borate, 1 mM EDTA).

(g) Western Blot

The method was used as previously described, Lee & Chang. (1996) J. Biol. Chem. 271. 10405-10412, with some modifications. Briefly. 5 μg of pCMV-TR2 was transiently transfected into T47D cells and lysed in RIPA buffer (10 mM sodium phosphate, pH 7.0. 150 mM NaCL, 2 mM EDT A. 1% (w/v) Nonidet P-40, 0.1% (w/v) SDS. 1% (w/v) sodium deoxycholate) with freshly adding proteinase inhibitors. The soluble protein was quantified using the Bio-Rad Protein Assay reagent (Bio-Rad Laboratories) and 50 μg soluble proteins were loaded onto SDS/10% PAGE and then transferred to Immobion-P transfer membrane (Millipore). After the blocking reaction overnight, the membrane was incubated with rat anti-PR polyclonal antibody (H-190. Santa Cruz) in PBS(−) containing 0.1% Skim milk for 2 h at room temperature. The membrane was washed and then incubated in 15 μCi 125I] of protein-A (DuPont NEN)/30 ml of PBS(−) containing 0.1% Skim milk for 1 h at room temperature. The western blots were autoradiographed and quantitated by using Phosphorlmager.

(h) Other Methods

RNA isolation and Northern blot analysis were performed as previously described. Young, et al., J. Biol. Chem. 273,20877-20885 (1998).

(2) Results

(a) Interaction between ER and TR2

The in vitro GST pull-down assay was applied to examine the interaction between the TR2 and ER. In vitro-translated, [35S]-labeled ER, AR, and RXRα proteins were incubated with GST or GST-TR2 bound on glutathione-Sepharose beads. After extensive washing, proteins were separated on an SDS/8% PAGE and visualized by using autoradiography. In vitro translated [35S]-ER and the androgen receptor (AR), but not retinoic A receptor a (RXRa), can interact well with the GST-TR2 that is bound to the glutathione-Sepharose beads, suggesting the TR2 can form a heterodimer with the ER. In vitro-translated [35S]-labeled TR2 protein was incubated with GST, GST-ER-N (aa 1-165 of ER), GST-ER-DBD (aa 123-340 of ER), GST-ER-LBD (aa 499-595 of ER), or GST-ER-F (aa 552-595 of ER) in the absence or presence of 1 μM E2. The partial LBD, (aa 499-595) or the F domain (aa 552-595) of the ER, but not the N-terminal domain (aa 1-165) or DBD (aa 123-340) of the ER, can interact with the [35S]-TR2 in the presence of 1 μM E2.

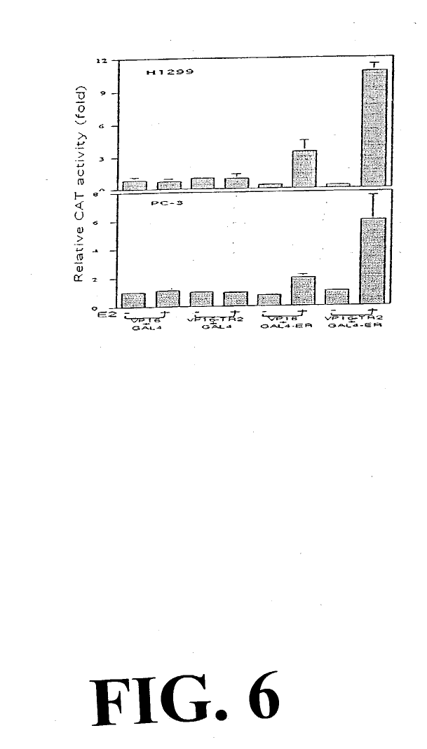

The in vivo mammalian two-hybrid system was applied to further confirm the interaction between the ER and TR2. A full-length TR2 was fused to the transcriptional activator VP16 (VP16-TR2) and then co-transfected with GAL4-DBD fused with ER-LBD (GAL4-ER, aa 282 to 595) and a GAL4-responsive CAT reporter (pG5-CAT) in H1299 cells. More specifically, H1299 cells and PC-3 cells were transiently co-transfected with 3 g of pSG5-CAT reporter plasmid, 3 μg of GAL4 or GAL4-ER expression plasmid, and 3 μg of VPI6 or VP16-TR2 expression-plasmid. Interaction was estimated by determining, CAT activity levels in the presence or absence of 108 M E2. Cells were also transfected with a β-galactosidase expression plasmid, pCMV-β-gal, as an internal control for transfection efficiency. As shown in FIG. 6, co-transfection of the parental vector pCMX-GAL4 with pCMX-VPI6 or VPI6-TR2 results in a low background in the presence or absence of 10 nM E2. While co-transfection of the pCMX-VPI6 with GAL4-ER showed some self-activation in the presence of 10 nM E2, a significant induction of CAT activity was observed only when cells were co-transfected with the VP16-TR2 and GAL4-ER in the presence of 10 nM E2, indicating that the E2 could promote the interaction between the GAL4-ER and VPI6-TR2. Similar results were obtained when the H1299 cells with prostate PC-3 cells were replaced (FIG. 6). Taken together, results from the in vitro GST pull-down and the in vivo mammalian two-hybrid assays provide strong evidence that the ER and TR2 can interact with each other in the presence of E2.

(b) ER Functions as Repressor to Repress the TR2-Mediated Transactivation.

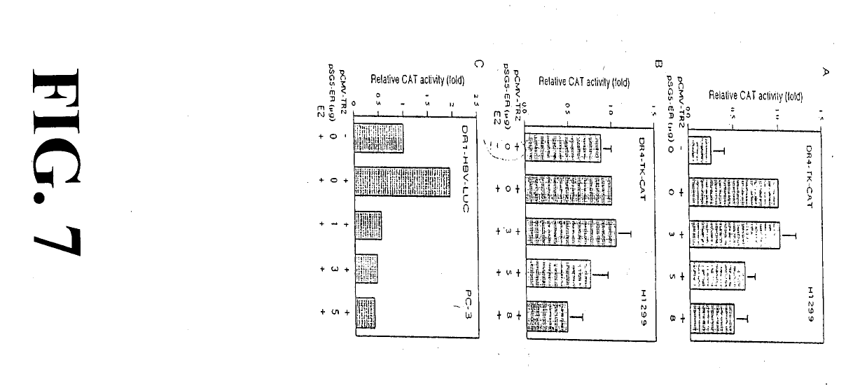

The CAT reporter assay was used to study the potential consequence of the formation of heterodimers between the TR2 and ER. Full-length ER and TR2 in eukaryotic expression vectors (pSG5-ER and pCMV-TR2) were co-transfected with a CAT reporter containing a TR2-response element (DR4-TK-CAT), Lee et al., J. Biol. Chem. 272, 12215-12220 (1997), in H 1299 cells. More specifically, H1299 cells were transiently cotransfected with 2 μg of DR4-TK-CAT reporter plasmid and 3 μg of TR2 expression plasmid together with increasing amounts of the ER expression vector. CAT activity was analyzed in the absence (FIG. 7A) or presence (FIG. 7B) of 10−8 M E2. Cells were also transfected with internal control reporter plasmid, pCMV-β-gal, as an internal control for transfection efficiency. Luciferase activity was then analyzed following manufacturer's instructions (Promega). As shown in FIGS. 7A and 7B, the CAT activity induced by the pCMV-TR2 could be repressed in a dose-dependent manner by co-transfection of the pSG5-ER in the absence (FIG. 7A) or presence (FIG. 7B) of E2. Similar results were obtained when the DR4-TK-CAT reporter with DR1-HBV-LUC were replaced, another TR2 response element (FIG. 7C). Other steroid receptors, such as PR and glucocorticoid receptor (GR), showed no suppressive effect. The data therefore suggests that the ER is able to function as a repressor to repress the TR2-mediated transactivation in H1299 cells.

(c) Suppression of ER Target Gene Expression by TR2

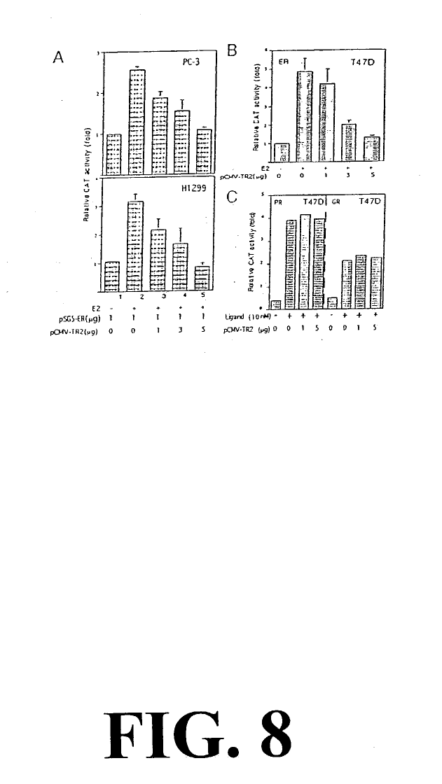

As the TR2 was able to suppress several genes that are regulated by the vitamin D receptor, thyroid hormone receptor, and retinoic acid receptor, Lin et al., J. Biol. Chem. 270,30121-30128 (1995), Lee et al., J Biol. Chem. 272, 12215-12220 (1997), Lee, et al., J: Biol. Chem. 274, 13437-13443 (1998), the potential reverse effects of the TR2 on the ER transactivation were determined. PC-3 cells and H1299 cells were co-transfected with 1 μg of ERE-CAT reporter plasmid and 1 μg of ER expression plasmid together with increasing amounts of the TR2 expression vector. Relative CAT activity was determined in the absence or presence of 10⁻⁸ M E2. As shown in FIG. 8A, the ERE-CAT activity was induced by transfection of the pSG5-ER in the presence of 10 nM E2 in PC-3 cells (lane 1 vs 2). Addition of the TR2 resulted in the suppression of ER transactivation in a dose-dependent manner (lane 2 vs 3-5). Similar suppression effects also occurred when the PC-3 cells were replaced with H1299 cells (FIG. 8A).

To eliminate the potential artificial effects caused by overexpression of exogenous ER, T47D cells were used as a model to test the TR2 suppression effect on the endogenous ER-mediated transactivation. T47D cells were transfected with 1 μg of ERE-CAT reporter plasmid together with increasing amounts of the TR2 expression plasmid. The CAT-activity was observed with the endogenous ER in the absence or presence of 10⁻8 M E2. All of the cells also were transfected with a galactosidase expression plasmid, pCMV-β-gal to normalize the transfection efficiency. As expected, the TR2 can still repress the endogenous ER-mediated ERE-CAT activity (FIG. 8B). For a control, the potential suppression effect of the TR2 on other steroid receptors-mediated transactivation was also tested, such as PR or GR. H1299 cells were co-transfected with 1 μg of MMTV-CAT reporter plasmid and 1 μg of PR or GR expression plasmid together with increasing amounts of the TR2 expression vector. Relative CAT activity was observed in the absence or presence of 10⁻⁸ M progesterone or glucocorticoid, respectively. AU of the cells also were transfected with a β-galactosidase expression plasmid, pCMV-β-gal to normalize the transfection efficiency. It was found that TR2 has little effect on the PR- or GR-mediated transactivation (FIG. 8C). Taken together, data from FIG. 8 clearly demonstrated that the TR2 could repress the E2-induced ER transactivation in various cell lines.

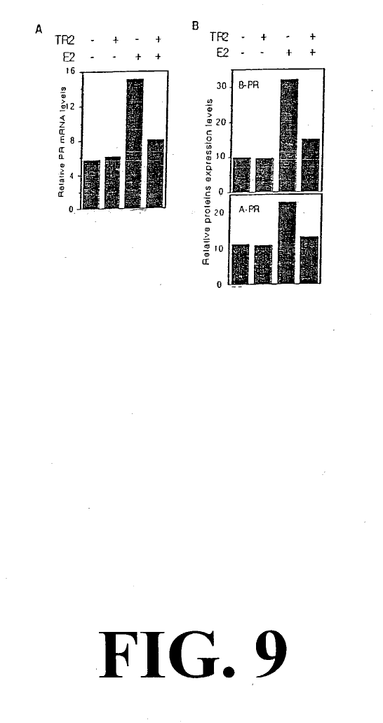

Next it was determined if TR2 could repress the ER endogenous target gene expression. PR was chosen because it is well studied as an E2-ER target gene in T47D cells. Misrahi et al., Biochem. Biophys. Res. Commun. 143, 740-748 (1987). After 24 h transient transfection of the pCMV-TR2 or parent vector pCMV, the T47D cells were treated with or without 10 nM E2 and cultured for another 24 h. Cells were then used for RNA isolation (Northern blot analysis) and lysate protein extraction (Western blot analysis). For RNA analysis, total RNAs were isolated, and Northern blots were performed using [32P] labeled PR cDNA probes. The membrane was autoradiographed. For Protein Western blot analysis, cells lysates were prepared and PR proteins were visualized by radioimmunoblot. The membrane was autoradiographed to Phosphorimager and was quantitated by ImageQuant software. As shown in FIG. 9A, the PR mRNA expression was induced about 3 fold after E2 treatment. Addition of the TR2 clearly repressed the expression of endogenous PR mRNA in the presence of 10 nM E2.

The Western blot analysis also clearly demonstrated that TR2 could repress the endogenous PR expression in T47D cells (FIG. 9B). Similar results were obtained when the T47D cells were replaced with another breast cancer cell line, MCF7. These in vivo TR2-mediated suppressive effects strongly support the above reporter assays and demonstrate that TR2 can function as a repressor to repress ER target genes in breast cancer cells.

(d) TR2 Prevents ER from Binding to its Target DNA

The EMSA using [32P]-labeled ERE as probe was applied to further dissect the mechanism of how the TR2 represses ER-mediated transactivation. 0.1 ng [32P] end-labeled ERE oligomers (4×108 dpm/μg) were incubated with in vitro translated TR2 or ER proteins in EMSA binding buffer and analyzed on a 5% acryamide gel containing 2.5% glycerol. 1 μg of Anti-ERa monoclonal antibody (C314) was used for supershifting. A 100-fold excess unlabeled ERE oligomer was used as a competitor. The specific ER-ERE band could be supershifted by adding ERα monoclonal antibody C-314. Addition of 100-fold unlabeled ERE oligonucleotides effectively eliminated this specific band. The intensity of this ER-ERE supershift decreased upon adding increasing amount of the TR2 in the absence or presence of 1 μM E2. Together, these results suggest that the TR2 might be able to repress ER target genes by preventing the ER from binding to its target DNA. As there is no TR2-ERE specific band and no extra shifted band formed upon the addition of the TR2 to the ER-ERE complex, the data can also rule out the possibility of the formation of a transcriptional inactivated TR2-ER-ERE complex.

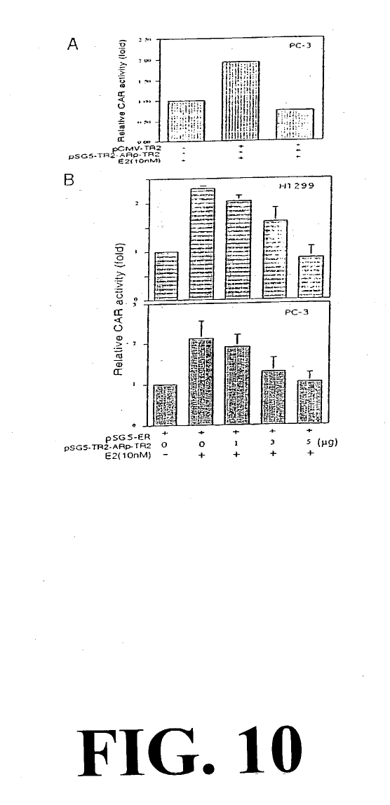

This conclusion was further confirmed by using the chimera receptor, TR2-androgen receptor (AR) ARp-TR2, which was generated by swapping TR2 proximal box (P-box) with that found in the AR. PC-3 cells were transiently co-transfected 2 μg of DR1-HBV-LUC reporter plasmid with 1 μg of pCMV, pCMV-TR2 or pSG5-TR2-ARp-TR2 in 35 mm dishes. Cells were treated with 10⁻⁸ M E2 after 24 h transfection. Luciferase activity was then analyzed following manufacturer's instructions (Promega). Cells also were transfected with a β-galactosidase internal control reporter to correct for transfection efficiency. It was found that the TR4 target DRI-HBV-LUC activity could be induced only by wild-type TR2 but not TR2-ARp-TR2 (FIG. 10A). The effect of TR2-ARp-TR2 on the ERE-CAT activity was tested. H1299 and PC-3 cells were transiently co-transfected with 1 μg of ERE-CAT reporter plasmid with 1 μg of ER expression plasmid together with increasing amounts of TR2-ARp-TR2 expression vector. CAT activity in the presence of 10⁻⁸ M E2. Cells also were transfected with β-galactosidase internal control reporter to correct for transfection efficiency. It was found that the ERE-CAT activity could be also repressed by TR2-ARp-TR2 in a dose-dependent manner in HI 299 and PC-3 cells (FIG. 10B). Together, these results support the GST pull-down assay and EMSA data by suggesting the possible mechanism of TR2 suppressive effect is through the interaction of ER with TR2-LBD, rather than through the TR2-DBD to compete out the binding between ER and ERE.

(e) Other Interactions.

Using similar procedures, the interactions between the TR2 receptor and the AR receptor were studied. It was found that the TR2 receptor can suppress transactivation by the AR receptor. Similarly, studies were undertaken to evaluate the interaction between the RXR receptor and the AR, and it was found that the RXR receptor can also suppress transactivation of the AR receptor. Hence, several mechanism exist to permit suppression of the AR receptor through co-suppression by an allied nuclear receptor.

### 2. Example 2

### Suppression of Estrogen Receptor-Mediated Transcription and Cell Growth by Interaction with TR2 Orphan Receptor

A) Materials and Methods

(1) Antibodies

ER rabbit polyclonal (H-184), ER mouse monoclonal (C314), and progesterone receptor (PR) rabbit polyclonal (HI90) were obtained from Santa Cruz Biotechnology. TR2 rabbit polyclonal (#1132) and mouse monoclonal anti-TR2 IgM antibody (G204) were described previously (14). Monoclonal anti-FLAG antibody (M2) was purchased from Sigma. Biotinylated secondary antibodies (goat-anti rabbit IgG and goat-anti mouse IgM) were from Vector Laboratories, Inc. (Burlingame, Calif.). AP-conjugated secondary antibodies (goat anti-rabbit IgG and goat anti-mouse IgM) were from Santa Cruz Biotechnology.

(2) Immunohistochemistry

Mammary glands were removed from 4-week-old virgins or 6-week-old lactating females of the 129SVEV×C57BL6 mouse strain (Lexicon Genetics, Inc., The Woodlands, Tex.). Tissues were fixed overnight, paraffin-embedded, and sectioned at 6-μm thickness. Sections were immersed in 0.01 M Sodium Citrate (pH 6.0) and microwaved for 3 cycles of 5 min at 700W. After antigen retrieval, sections were blocked with 1% hydrogen peroxide in methanol for 15 min, then with 20% normal goat serum in TBS for 25 min. Sections were incubated with the primary antibodies (1:100) followed by secondary antibodies (1:200) in TBS containing 1% BSA. Staining was visualized by incubation of Vectastain ABC solution (Vector Laboratories, Inc.) followed by development with DAB peroxidase substrate kit (Vector Laboratories, Inc.). Sections were counterstained with hematoxylin, dehydrated, cleared, and mounted with Permount (Fisher Scientific, Fair Lawn, N.J.). For negative control sections, primary antibody was replaced with normal rabbit IgG.

(3) Constructs

The pCMV-TR2, pGEX-3×-TR2, and pCMX-VPI6-TR2 were constructed by insertion of full-length TR2 cDNA (Chang, C. et al. (1989) Biochem. Biophys. Res. Commun. 165, 735-41; Chang, C. et al. (1988) Biochem. Biophys. Res. Commun. 155, 971-7) into individual vectors. The doxycycline-inducible expression vector pBIG2i bearing hygromycin B resistance gene was a gift from Dr. Jay Reeder (University of Rochester, N.Y.) (Strathdee, C. et al. (1999) Gene 229, 21-9). pBIG2i and pBIG2i-FLAG-TR2 were used for generating MCF7-pBIG and MCF7-TR2 stable clones, respectively. The GAL4-ER (aa 282-595) and pCMV-mERβ were gifts from Dr. Hinrich Gronemeyer (Strasbourg, France) and Vincent Giguére (McGill University, Quebec, Canada), respectively. To construct GST-ER fragments, ER cDNA fragments were released from pSG5-ER (Green, S. et al. (1988) Nucleic Acids Res. 16, 369) using adequate restriction enzymes and inserted into the pGEX vector series (Amersham Pharmacia) to produce pGEX-3X-ER-#1 (aa 1 to 165), pGEX-2T-ER-#2 (aa 123-340), pGEX-2T-ER-#3 (aa 312-595), pGEX-3X-ER-#4 (aa 552-595), pGEX-2T-ER-#5 (aa 123-312), and pGEX-2T-ER-#6 (aa 312-340). The pGEX-KG-TR2-#1, #2, and #3 plasmids were constructed by insertion of PCR-generated cDNA fragments corresponding to aa 1-112, aa 88-196, and aa 179-603, respectively, into pGEX-KG vector (Guan et al., (1991), Analytical Biochemistry 192:362-4, which is herein incorporated by reference at least for material related to plasmids and assays, including sequence). pcDNA3-TR2-fl AS and pIRES-TR2-N AS were constructed by insertion of opposite orientation of cDNAs encoding full length and N terminal (aa 1-112) into pcDNA3 (Invitrogen) and pIRES (Clontech), respectively.

(4) Transient transfection

Transfections and chloramphenicol acetyltransferase (CAT) assays were performed using the calcium phosphate precipitation method, as described previously (Yeh, S. et al. (2000) Proc. Natl. Acad. Sci. USA 97, 11256-61). CAT reporter plasmids containing one copy of estrogen response element (ERE-CAT), or mouse mammary tumor virus (MMTV-CAT) were used as indicated. Also, a β-galactosidase expression plasmid, pCMV-β-gal, was used for transfection efficiency.

(5) Co-immunoprecipitation

MCF7 cells plated on 100-mm dishes were solubilized in 1 ml RIPA buffer containing 0.5% NP-40 and protease inhibitors. Immunoprecipitation was performed using rabbit anti-ER antibody (1:1000) (H-184) and then analyzed by Western blotting with anti-ER (1:1000) (H-184) or anti-TR2 (1:1000) (G204) antibodies, followed by incubation with AP conjugate goat anti-rabbit or rabbit anti-mouse IgM antibodies, and visualized with AP conjugate kit (Bio-Rad).

(6) GST Pull-Down Assay

GST alone and GST fusion proteins were purified by Glutathione-Sepharose 4B beads as instructed by manufacturer (Amersham Pharmacia). The pull-down assay was performed with 5 μl of in vitro-translated [³⁵S]-labeled proteins as described previously (Yeh, S. et al. (2000) Proc. Natl. Acad. Sci. USA 97, 11256-61).

(7) Electrophoretic Mobility Shift Assay (EMSA)

EMSA was carried out as described previously (Lee, Y. et al. (1999) Proc. Natl. Acad. Sci. USA 96, 14724-9) with some modifications. Human complement C3 ERE (containing one imperfect palindromic inverted repeat: 5′-AGGTGGCCCTGACCC-3′) end-labeled with γ-[³²P]-ATP was used as probe. ER and TR2 were in vitro translated by TNT system as instructed by manufacturer (Promega). Reactions were performed in 20 μl of EMSA binding buffer (10 mM HEPES/pH 7.9, 100 mM KCl, 1 mM Dithiothreitol, 0.5 mM EDTA, 2.5 mM MgCl₂, and 6% glycerol). For the antibody supershift analysis, 1 μl of the monoclonal anti-ERα antibody (C-314) was used. The protein-DNA complexes were analyzed on a 5% polyacrylamide native gel containing 2.5% glycerol in 1×TBE.

b) Results

(1) TR2 is Expressed in Mammary Epithelial Cells and Breast Cancer Cell Lines

The studies of TR2 tissue distribution indicate that TR2 is expressed in many tissues with higher expression in brain and male reproductive organs (Chang, C. et al. (1989) Biochem. Biophys. Res. Commun. 165, 735-41; Chang, C. et al. (1988) Biochem. Biophys. Res. Commun. 155, 971-7; Young, W. et al. (1998) J. Biol. Chem. 273, 20877-85; Lee, H. et al. (1996) J. Biol. Chem. 271, 10405-12). The expression of TR2 in mammary glands was examined. Using an immunohistochemical staining with anti-TR2 antibody, it was found that 30 to 40% of luminal cells within the mammary ducts of 4-week-old female mice were TR2-positive (These data were collected as follows: (A) Immunohistochemical staining of TR2 and ER in mouse mammary glands. Mammary glands were removed from 4-week-old virgins of the 129SVEV×C57BL6 mouse strain (Lexicon Genetics, Inc.) and paraffin-embebbed as described under “Materials and Methods”. The adjacent sections were stained with polyclonal rabbit anti-TR2 (#11132) and anti-ER antibodies (H-184), followed by treatment with goat anti-rabbit biotinylated secondary antibody. Immunohistochemical staining was visualized by incubation in Vectastain ABC solution and DAB peroxidase substrate kit and counterstained with hematoxylin. For negative control section, the primary antibody was replaced with normal rabbit IgG. Positive and negative staining shows brown color and blue color, respectively. Cells that are identical between adjacent sections and show both TR2- and ER-positive were determined. (Magnification: 400×). (B) TR2 immunohistochemical staining in mammary glands dissected from 6-week-old lactating females. Methods used are the same as described in (A). (Magnification: 400×). (C) Northern blotting for TR2 mRNA expression in cancer cell lines. 20 mg of total RNA was extracted from cancer cells. A TR2 cDNA encoding LBD (aa 179-603) was random-primed labeled with a-[32P]-dCTP and used as probe. Northern hybridization was performed with Rapid-hyb buffer (Amersham Pharmacia) according to manufacturer's instructions. 28S ribosome RNA was stained with 0.04% methylene blue in sodium acetate (pH 5.0) for RNA integrity and quantity control). In these cells, TR2 was clearly detected in both cytosol and nucleus. Some of the stroma cells in fat pads were also TR2-positive, while the flattened basal cells around the ducts and cap cells surrounding TEBs were likely all TR2-negative. When the adjacent section was stained with ERα antibody, it was observed that the expression pattern of ER was quite similar to that of TR2 except that most of the ER staining was mainly located in the nucleus. It has been known that during the lactation stage, mammary glands undergo intense differentiation to form lobuloaveolar structures and the ER expression cells are known to be highly increased (Saji, S. et al. (2000) Proc Natl Acad Sci USA 97, 337-42; Saji, S. et al. (2001) Endocrinology 142, 3177-86). Therefore, whether the TR2 expression is increased in lactating glands was determined. TR2 staining was observed to be obvious and thoughout mammary luminal cells as compared to that on the control section stained with normal rabbit IgG.

ER is known to be an important regulator for breast cancer development as two thirds of breast tumors contain a functional ER that mediates estrogen responsiveness for cell growth. To determine whether TR2 is expressed in ER-positive breast cancer cells, Northern blotting was employed. TR2 transcripts around 2.5 kb were expressed at different levels in three breast cancer cell lines (MCF7, T47D, and ZR-75-1), which were well documented as containing both ERα and ERβ. TR2 could also be detected in prostate cancer PC-3 and LNCaP cells.

(2) TR2 Specifically Suppresses ER-Mediated Transcription

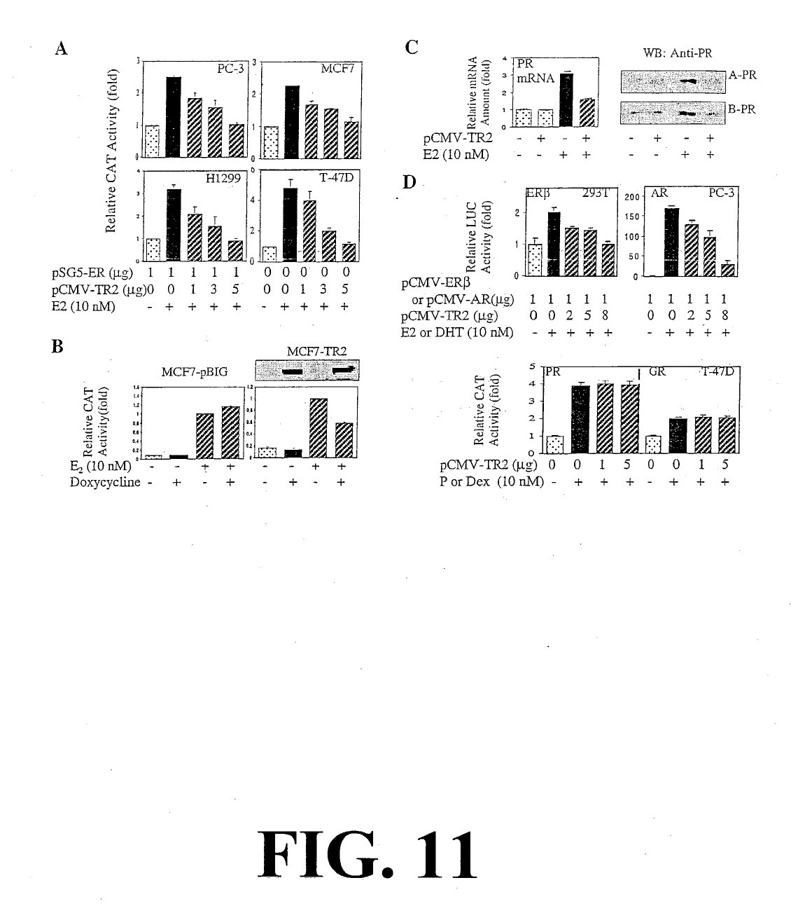

TR2's influence on ER function was observed. Using either ERE-CAT or ERE-luciferase reporter systems, TR2 consistently suppressed either exogenous (in PC-3 and h1299 cells) or endogenous (in MCF7 and T47D cells) ER in a dose-dependent manner (FIG. 1 IA). Using a clone of MCF7-TR2 cells, which were stably transfected with a doxycycline-inducible pBIG2i-FLAG-TR2 plasmid, it was found that ER was suppressed by doxycycline-induced TR2. By contrast, ER was not suppressed by doxycycline treatment in the MCF7-pBIG cells which were stably transfected with the pBIG2i parent vector (FIG. 11B). To rule out the artificial effects linked to foreign reporters as demonstrated in FIGS. 11A & B, PR expression, an endogenous target gene of ER, was examined. As shown in FIG. 11C, TR2 could repress E2-induced PR expression at mRNA and protein levels in T47D cells and MCF7 cells (data not shown). TR2 could also suppress the basal level of ER transcription in the absence of E2. For examining specificity, the effect of TR2 on other classical steroid receptors was analyzed. As shown in FIG. 11D, while TR2 could also suppress ERβ- and AR-mediated transcription in HEK293 (no detectable ERα) and PC-3 cells, respectively, TR2 has marginal effect on the PR- or glucocorticoid receptor (GR)-mediated transcription in T47D cells. Together, results from FIG. 11 demonstrate that TR2 can suppress ERα-mediated transcription and these suppression effects are receptor-specific. (3) TR2 Physically Associates with ER

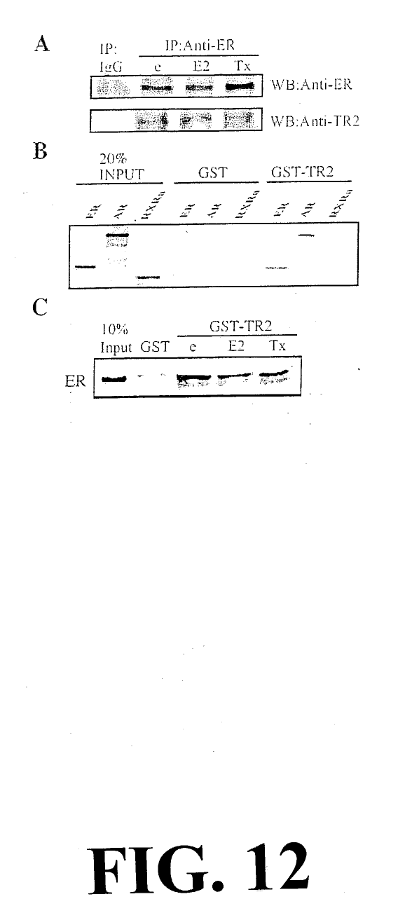

To investigate whether TR2 and ER are physically associated, co-immunoprecipitation and GST pull-down assays were carried out for examination of in vivo and in vitro interaction. Cell extracts from MCF7 cells treated with ethanol, E2, and tamoxifen were co-immunoprecipitated with anti-ER antibody (H 184). Immunocomplexes were then Western blotted with anti-TR2 antibody (G204). As shown in FIG. 12A, TR2 existed in the ER-immunocomplexes in the presence of ethanol, E2, or tamoxifen. Using a GST pull-down assay, GST-TR2 fusion protein could directly interact with in vitro translated [³⁵S]-labeled ER and AR, but not RXR(X (FIG. 12B). For testing ligand effects on the ER-TR2 binding, not much difference was found among different treatments (FIG. 12C). Collectively, these results suggest that ER and TR2 are directly associated with each other in a ligand-independent manner.

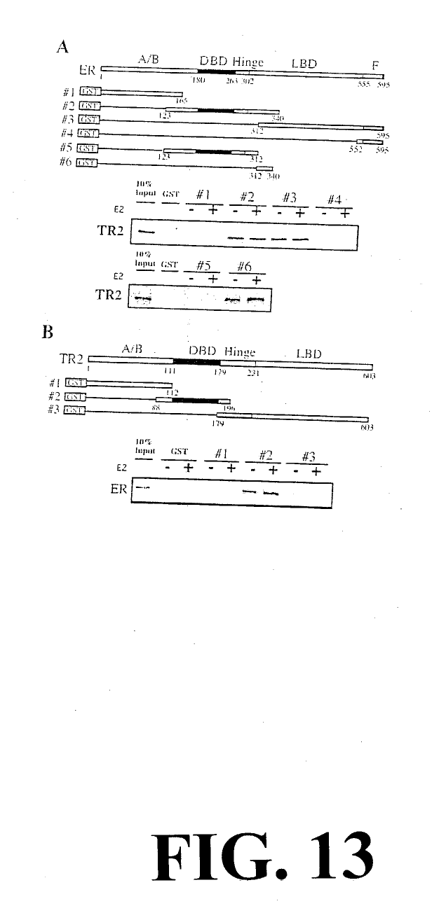

To dissect the TR2 interaction domain on ER, six ER peptides fused with GST were tested in GST pull-down assays. As shown in FIG. 13A, GST-ER-#2 (aa 123-340) and GST-ER-#3 (aa 312-595), but not GST-ER-#I (aa 1-165) and GST-ER-#4 (aa 552-595) can interact with TR2 in the presence or absence of E2. Furthermore, GST-ER-#6 (aa 312-340), the overlapping region between GST-ER-#2 and -#3, but not GST-ER-#5 (aa 123-312), showed positive interaction with TR2, indicating that the ER-#6 domain is responsible for this interaction. On the other hand, three GST-fused TR2 fragments, GST-TR2-#1, -#2, and -#3, corresponding to N-terminus (aa 1-112), DBD (aa 88-196), and LBD (aa 179-603), respectively, were also examined to locate the ER-binding site. As shown in FIG. 13B, GST-TR2-#2, but not GST-TR2-#1 or -#3, was responsible for binding to ER.

(4) Direct Association is Required for TR2-Mediated Suppression on ER

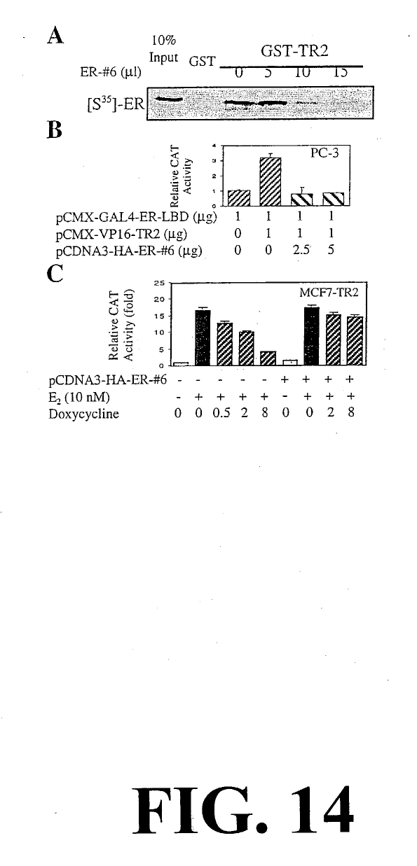

In order to determine whether ER-#6 can serve as an interaction blocker, the interference with ER-TR2 binding by ER#6 was tested by GST pull-down assay and mammalian two-hybrid system where in vitro translated HA-ER-#6 and pcDNA3-HA-ER-#6 plasmid were introduced, respectively. First, the interaction of GST-TR2 with [³⁵S]-labeled ER was inhibited by increasing amounts of HA-ER-#6 peptide (FIG. 14A). Second, GAL4-ER can interact with VP16-TR2 in the presence of E2, according to the induction of CAT activity, and this ER-TR2 interaction was suppressed when co-transfecting with pcDNA3-HA-ER-#6 (FIG. 14B). Thus, based on these results, ER-#6 is able to be an interaction blocker. Next, to determine whether direct association is required for TR2 to suppress ER, pcDNA3-HA-ER-#6 was applied in an ERE-CAT reporter gene assay. As shown in FIG. 14C, the E2-induced ER transcription was significantly repressed by the doxycycline-induced TR2 in a dose-dependent fashion in MCF7-TR2 cells. Addition of ER-#6 was then capable of reversing this suppression, suggesting that TR2 suppresses ER through direct interaction.

(5) The Biological Significance of TR2 on ER Activity

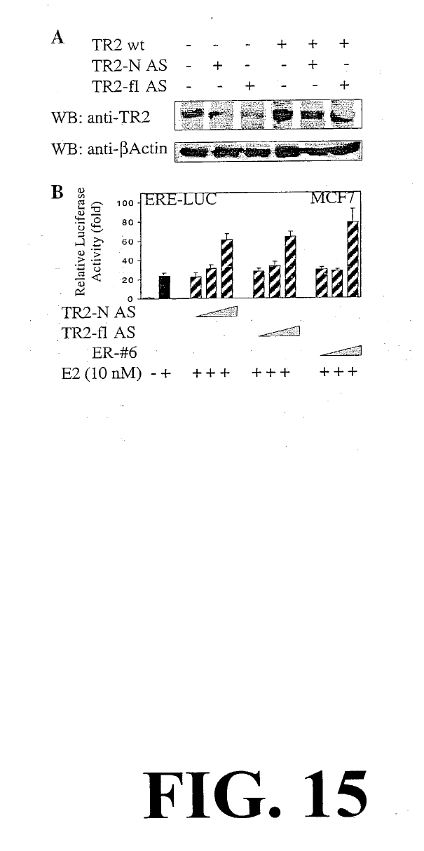

Antisense TR2 expression plasmids, pcDNA3-TR2-fl AS and pIRES-TR2-N AS, were assessed in an ERE-luciferase assay to determine whether blockage of endogenous TR2 expression might significantly enhance ER activity in MCF7 cells. First, using Western blotting with anti-TR2 antibody (G204), those two antisense constructs were proven to be capable of reducing endogenous TR2 expression as well as overexpressed TR2 (FIG. 15A). This reduction of endogenous TR2 by antisense plasmids resulted in an increase in ER transcription in a dose-dependent manner (FIG. 15B), indicating that endogenous TR2 normally suppresses ER in MCF7 cells. Meanwhile, the transfection of the interaction blocker, pcDNA3-HA-ER-#6, at a higher amount was also able to enhance ER transcription.

(6) ER DNA-Binding and Homodimeric Formation are Disrupted By Associating with TR2

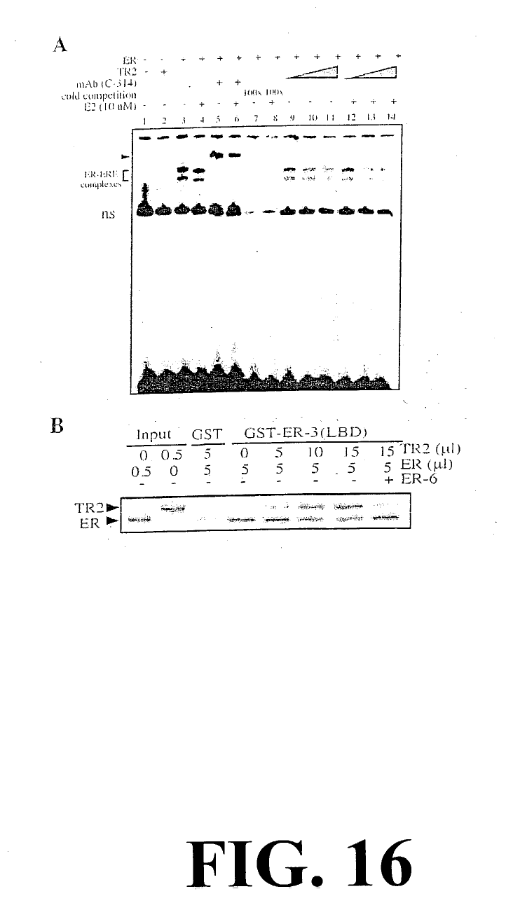

To elucidate the molecular mechanisms by which ER was suppressed by interacting with TR2, ER expression, stability, nuclear translocation, DNA binding, and interaction with coregulators were tested. It was found that overexpression of TR2 did not influence ER expression, stability, or nuclear translocation (data not shown). Using GST pull-down assay and mammalian two-hybrid assay, TR2 did not affect the binding between ER and some coregulators such as SRC-1, TIF-II, and ARA70. After ruling out these mechanisms, TR2 would be thought to mainly influence ER on DNA binding. Using the EMSA assay as shown in FIG. 16A, two specific ER-ERE bands could be detected (lane 3 and 4) and were supershifted by ER antibody (C314) (lanes 5 and 6, indicated as an arrowhead). Addition of 100-fold molar excess of unlabeled ERE could effectively eliminate these specific bands (lanes 7 and 8). Interestingly, the intensity of these ER-ERE complexes were decreased upon addition of increasing amounts of TR2 in either the absence (lanes 9-11) or the presence of 10 nM E2 (lanes 12-14). Since no ERE-TR2 specific band (lane 2) and no extra supershifted band formed as TR2-ER-ERE complexes (lanes 3-4 vs 9-14) were found, it can be concluded that TR2 interacts with ER resulting in ER dissociating from binding to DNA. Indeed, the competition assay (FIG. 16B) showed that the ER homodimer formation, as illustrated by the interaction between GST-ER-LBD and [³⁵S]-ER, was interrupted by the presence of TR2 and, conversely, heterodimeric formation of ER-TR2 was increased along with the increasing amounts of TR2. Furthermore, the reduction of ER homodimerization by TR2 could be rescued when the ER-#6 peptide which blocked TR2 from interacting with ER, was added (FIG. 16B). Taken together, FIG. 16 suggests that TR2 can suppress ER-mediated transactivation via the formation of TR2-ER heterodimers that reduce the formation of the ER homodimer and cause ER to associate from ERE.

(7) TR2 Suppresses E2ER-induced G1/S transition and Causes Growth Arrest

As previous reports revealed that E2/ER plays important roles in the stimulation of mammary gland and breast cancer growth (Imagawa, W. et al. (11990) Endocr Rev 11, 494-523; Katzenellenbogen, B. et al. (1997) Breast Cancer Res. Treat. 44, 23-38; Dickson, R. et al. (1987) Endocr. Rev. 8, 29-43), whether the suppression of ER by TR2 can modulate breast cancer cell growth was tested. The MTT assay (FIG. 117A) showed that addition of E2 apparently induced cell growth in both MCF7-pBIG (64-fold) and MCF7-TR2 (101-fold) cells, as compared to both cells treated with ethanol (2.7- and 15.2-fold, respectively). As expected, the doxycycline treatment caused growth arrest in the MCF7-TR2 cells (down to 11.1-fold), but not in the MCF7-pBIG cells without TR2 induction (75.2-fold). These results indicate that E2-induced cell growth was inhibited by TR2. Meanwhile, it was also observed that the cell size of MCF7-TR2 became larger after 3 days of doxycycline treatment, but did not occur in MCF7-pBIG cells.

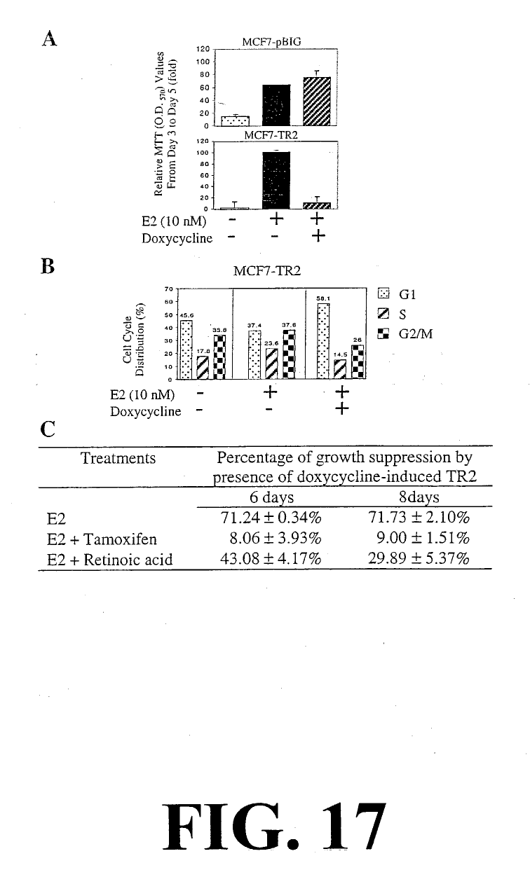

It has been reported that E2/ER plays a role in promoting G11/S transition to enhance cell proliferation and suppression of E2/ER signaling such as when treated with antiestrogens can cause cell growth arrest at the GI phase (46). To determine whether TR2 can interrupt E2/ER-induced G1/S transition, the cell cycle profile was obtained from flow cytometry using MCF7-TR2 cells which were treated with ethanol, E2 and doxycycline for 3 days. The results showed that E2 can enhance G1/S transition (G1: from 45.6% to 37.4%; S: from 17.8% to 23.6%) (FIG. 17B). In contrast, TR2 expression inhibited the E2-induced G1/S transition, leading to G1 arrest (G1: from 37.4% to 58.1%; S: from 23.6% to 14.5%).

To further confirm that TR2-induced cell growth inhibition requires the E2/ER signaling, antiestrogen tamoxifen was applied to see if TR2-induced cell growth inhibition was diminished. As shown in FIG. 17C, in the presence of E2, 71-72% of MCF7-TR2 cell growth was suppressed by TR2 after 6- and 8-day treatments, as compared to cells without doxycycline. Under 5 μM tamoxifen treatment, which inhibited E2/ER-mediated growth signaling in MCF7-TR2 cells, only 8-9% of cells could be further suppressed by TR2, implying that ER signaling is important for TR2-induced cell growth inhibition. In the control experiment, TR2 can further suppress the growth of all-trans retinoic acid treated cells to 29-45%. Together, FIG. 17 demonstrates that TR2 could suppress E2/ER-induced cell growth and G1/S transition via suppression of ER signaling.

In mammary glands, TR2 staining was observed to be heterogeneous in the ductal epithelia and TEBs as well as the stroma cells within the connective tissues. In contrast, the highly proliferating cap cells comprising a monolayer of stem cells forming the leading edge of TEBs were likely all TR2-negative. The expression pattern of TR2 in mammary glands observed is highly correlated to that of ER described previously (Zeps, N. et al. (1998) Differentiation 62, 221-6; Haslam, S. et al. (1992) J Steroid Biochem Mol Biol 42, 589-95). Also, the co-localization of both receptors could be seen in many luminal cells from adjacent sections and in three ER-positive breast cancer lines. These results are consistent with the biological function of TR2 being related to ER-mediated regulation in the growth and differentiation of mammary glands. ER functions as a ligand-dependent transcription factor and its activity is known to be highly regulated by the balance between coactivators and corepressors inside the cells. It has been well documented that ER expression and activity in mammary glands are dramatically altered during different developmental phases (Saji, S. et al. (2000) Proc Natl Acad Sci USA 97, 337-42; Saji, S. et al. (2001) Endocrinology 142, 3177-86; Haslam, S. et al. (1992) J Steroid Biochein Mol Biol 42, 589-95). For instance, during the lactation stage, mammary glands undergo intense differentiation, such as formation of lobuloaveolar structures for milk production, meanwhile, the ER expression levels and cell numbers are observed to be apparently elevated (Saji, S. et al. (2000) Proc Natl Acad Sci USA 97, 337-42; Saji, S. et al. (2001) Endocrinology 142, 3177-86). However, ER in lactating glands fails to respond to estrogen to elicit PR mRNA expression in contrast to tissues from other stages, suggesting that an alteration in the transcriptional regulation of ER occurs (Shyamala, G. et al. (1990) Endocrinology 126, 2882-9). In this case, it is conceivable that the transcriptional silence of ER can be due to the prominent expression of corepressors in lactating glands. It is possible that TR2, a suppressor of ER, can play such a role as disclosed herein TR2 expression was increased in mammary glands at lactation stage and that TR2 inhibited estrogen-dependent PR mRNA expression in breast cancer cells (FIG. 11C).

TR2 was originally identified as a transcriptional factor that can modulate many target genes' expression via binding to the TR2 response elements (Chang, C. et al. (1989) Biochem. Biophys. Res. Commun. 165, 735-41; Chang, C. et al. (1988) Biochem. Biophys. Res. Commun. 155, 971-7; Lin, T. et al. (1995) J. Biol. Chem. 270, 30121-8). It is dislosed herein that TR2 functions as a repressor of ER-mediated transcription via direct protein-protein interaction (FIGS. 11 and 12) since the interaction blocker, ER-#6, an ER fragment (aa 312-340) responsible for TR2 binding (FIG. 13), was able to rescue ER from suppression by TR2 (FIGS. 14 and 15). Administration of antisense TR2 that enhanced ER transcription in MCF7 cells implies that endogenous TR2 normally down-regulates ER signaling (FIG. 15). TR2 neither affects ER expression levels or the nuclear translocation nor the interaction with some coactivators, such as SRC-1a, TIF-II, and ARA70 (data not shown). Data from EMSA (FIG. 16A) disclosed herein clearly demonstrate that the suppression of ER by TR2 is due to interruption of ER binding to DNA and this dissociation is caused by disruption of ER homodimers with the formation of non-functional TR2-ER heterodimers (FIG. 15B). It has been known that ER binds ERE as a homodimer in vivo based on a stoichiometry of a 2:1 ratio of ER to ERE in the majority of studies, suggesting that the homodimeric formation of ER is important and more stable for DNA-binding (See review in (Klinge, C. et al. (2001) Nucleic Acids Res 29, 2905-19)). Similarly, the disruption of ER homodimers through the interaction with truncated estrogen receptor product-1 (TERP-1) also shows an interruption of ER-ERE binding, resulting in the suppression of ER-mediated transcription (50). By contrast, a tumor suppressor, P53, suppresses ER through interfering with the DNA binding without affecting the dimerization (Liu, G. et al. (1999) Biochem. Biophys. Res. Commun. 264, 359-64). However, the mechanism by which p53 suppresses ER remains unclear.

The TR2 binding site on ER (aa 312-340) covers the region spanning from helix I to part of helix 3 within the N-terminal of the ER ligand binding domain (LBD), where no critical amino acid is responsible for hormone binding and dimerization (Tanenbaum, D. et al. (1998) Proc Natl Acad Sci USA 95, 5998-6003). This binding region for TR2 is different from the region, known as the AF-2 domain, for most other ER coactivators, such as SRC-1 and the related p160 family, which contain the signature motif of the NR box (LXXLL) responsible for interacting with ER in the presence of ligands (Heery, D. et al. (1997) Nature 387, 733-6). The AF-2 interaction surface is composed of the critical amino acids in helix 3, 4, 5, and 12, and, upon ligand binding, forms a hydrophobic cleft where helix 12 is positioned over the ligand-binding pocket providing a surface for those coregulators binding (Brzozowski, A. et al. (1997) Nature 389, 753-8; Shiau, A. et al. (1998) Cell 95, 927-37). The different binding sites between TR2 and ligand-dependent coactivators on ER is consistent as an explanation for the disclosed results showing that the ER-TR2 interaction was ligand-independent, as demonstrated by co-immunoprecipitation and GST pull-down assay (FIGS. 12 & 13), and that TR2 did not interfere with ER interacting with those coactivators. Consistent with this phenomena, an antiestrogen, tamoxifen, did not affect their interaction as shown in FIGS. 12A & C. It is also consistent with the finding that a signature motif, LXXLL, located on the TR2 LBD (aa 547-551) is not required for ER interaction since the ER binding site is located on the TR2 DBD (FIG. 13B). This phenomenon has also been demonstrated by Tanenbaum, et al (Delage-Mourroux, R. et al. (2000) J Biol Chem 275, 35848-56). They found that REA, a repressor of ER, interacts with ER through a ligand-independent fashion without involving the LXXLL motif of REA and, on the other hand, helix 12 of ER is not necessary for this interaction. However, they showed that the integrity of the LXXLL motif is still important for REA to perform its suppressive effect on ER, although it is not required for interaction.

It has long been known that the beneficial effects of antiestrogens on the ER-positive breast tumors is probably due to blockage of E2/ER-mediated cell growth (Group, E. B. C. T. C. (1998) Lancet 351, 1451-67). However, few of ER suppressors have been identified and characterized (Montano, M. et al. (1999) Proc. Natl. Acad. Sci. USA 96, 6947-52) and the detailed suppression mechanisms also remain largely unknown. Early reports suggested several possible mechanisms including: 1) the interference of the binding capacity of ER homodimers to ERE, such as P53 (Liu, G. et al. (1999) Biochem. Biophys. Res. Commun. 264, 359-64) and TERP-1 (Resnick, E. et al. (2000) J Biol Chem 275, 7158-66); 2) competition with coactivators for binding to ER, such as SHP (58), DAX-1 (Zhang, H. et al. (2000) J Biol Chem 275, 39855-9), TERP-1 (Resnick, E. et al. (2000) J Biol Chem 275, 7158-66), and REA (Delage-Mourroux, R. et al. (2000) J Biol Chem 275, 35848-56); 3) recruitment of HDACs to ER, such as metastatic-associated protein 1 (Mazumdar, A. et al. (2001) Nat Cell Biol 3, 30-7), and SMRT (Smith, C. et al. (1997) Mol Endocrinol 11, 657-66). Disclosed herein is another family of repressors, related to TR2 and interaction with the TR2 binding domain of ER, functioning through the formation of non-functional ER-TR2 heterodimers that result in ER dissociating from ERE.

ER is known to function as a modulator to regulate the function of other nuclear receptors, such as TR, RAR, and RXR, through protein-protein interaction (Lee, S. et al. (1998) Mol. Endocrinol. 12, 1184-92). ER also interacts with and suppresses proapoptotic forkhead transcription factor transcription activity in the presence of estrogen (Schuur, E. et al. (2001) J Biol Chem 276, 33554-60). Also disclosed herein ER could suppress TR2-mediated transcription in a ligand-independent manner. This suppression was not mediated via interruption of TR2 DNA binding although the ER interaction site is located on TR2 DBD.

FIG. 17 demonstrates that TR2 can suppress E2/ER-induced GI/S transition and cause cell growth inhibition in MCF7-TR2 cells where TR2 could be induced by treatment with doxycycline. This growth suppression is suggested to mainly go through suppression of ER signal since TR2 lost its suppressive effect on cell growth in the presence of tamoxifen (FIG. 17C). TR2 could additionally mediate growth inhibition through pathways independent of ER. Earlier studies have shown that TR2 induction is involved in neuronal differentiation in mouse P19 stem cells stimulated by either retinoic acid or CNTF (Young, W. et al. (1998) J. Biol. Chem. 273, 20877-85; Lee, C. et al. (2000) Biochem Pharmacol 60, 127-36). As TR2 is located in chromosome 12q22, a known region frequently deleted in various tumors including testicular and ovarian germ cell tumors (Faulkner, S. et al. (2000) Gynecol. Oncol. 77, 283-8; Murty, V. et al. (1996) Genomics 35, 562-70), linkage of TR2 as one of the tumor suppressor candidates that can negatively regulate cell growth is expected.

### 3. Example 3

### Modulation of Estrogen Receptor-Mediated Transactivation by Orphan Receptor TR4 in MCF-7 Cells

The human testicular orphan receptor 4 (TR4) is a member of the nuclear receptor superfamily that shows a broad tissue distribution with higher expression in the nervous system and male reproductive tract. TR4 functions as a transcriptional modulator that controls various target genes via binding to the DNA hormone response elements. Disclosed herein instead of direct binding to hormone response elements for gene regulation, TR4 can also go through direct protein-protein interaction to repress estrogen receptor (ER)-mediated transactivation. Electrophoretic mobility shift and GST pull-down clearly demonstrate that the direct interaction between TR4 and ER will inhibit the homodimerization of ER and interrupt/prevent ER binding to the estrogen response element. The consequence of these events can then result in the suppression of ER target genes, such as cyclin D1 and pS2 and inhibition of ER-mediated cell proliferation in the MCF-7 cells stably transfected with TR4.

a) Materials and methods

(1) Plasmids

pCMV-TR4, pSG5-ER, pSG5-PR, pET-14b-TR4, pGEX-3×-TR4, pERE-CAT, and pMMTV-LUC were reported previously (Young, W. et al. (1997) J. Biol. Chem. 272(5) 3109-16; Lee, Y. et al. (1999) Proc. Natl. Acad Sci. USA. 96(26) 14724-9; Yeh, S. et al. (1996) Proc. Natl. Acad. Sci. USA. 93(11) 5517-21), and pBIG-2i and pCMV-mERβ (pBIG-2i, Strathdee et al., (1999), Gene 229:21-29) and pCMVmER, Tremblay et al., (1999) Molecular Cell, 3:513-19, both of which are herein incorporated by reference at least for material related to plasmids and assays, including sequence. For the GST fusion constructs, pGEX-3X-TR4-N was made by cloning the SpeI/AatII fragment from pGEM-T EASY-TR4 into the SmaI site of pGEX-3X. pGEX-3X-AC-TR4 was made by removing HindIII/SacI fragment of pGEX-3X-TR4. pGEX-3X-TR4-LBD was made by inserting the PCR-generated (aa 224-615) fragment of the human TR4 cDNA in the pGEX-3×vector. pGEX-2T-ER-LBD was made by inserting the ER cDNA fragment (aa 312-595) into pGEX-2T. The expression plasmid pCMV ΔC-TR4 was generated by deleting the PstI fragment of pCMV-TR4. The pBIG-2i-TR4 was made by inserting the SpeI/NotI fragment of pGEM-T EASY-TR4 into SpeI/NotI site of pBIG-2i vector. All plasmids were verified by restriction enzyme analysis and DNA sequencing.

(2) Cell Culture and Transfection

H1299 and MCF-7 cells were maintained in Dulbecco's modified eagle medium (DMEM) containing penicillin (25 units/ml), streptomycin (25 μg/ml), and 10% fetal calf serum (FBS). Transfections were performed using the calcium phosphate precipitation method, as described previously (Lee, Y. et al. (1999) Proc. Natl. Acad Sci. USA. 96(26) 14724-9). Briefly, 3×10⁵ cells were plated on 60-mm dishes for 24 h before transfection, and the medium was changed to DMEM with 10% charcoal/dextran-stripped FBS. H1299 cells were transfected with an ER expression plasmid (pSG5-ER or pCMV-mERβ, ERE-chloramphenicol acetyltransferase (ERE-CAT) or pSG5-PR and MMTV-Luciferase reporter plasmid, and a TR4 expression plasmid (pCMV-TR4) ERZ-CAT Bradshaw et al., (1991) J. Biol. Chem. 266:16684-16690 and pCMVB-gal and PSV-RL from promega, all of which are herein incorporated by reference at least for material related to plasmids and assays, including sequence.) MCF-7 cells were only transfected with ERE-CAT reporter and TR4 expression plasmid. For all transfection experiments, pCMVβ-gal (CAT assay) or pSV-40 RL (Luciferase assay) was used as an internal control for transfection efficiency and the total amount of transfected DNA was adjusted to 12 μg with pCMV. After 24 h transfection, the media was changed again, and the cells were treated with 10 nM 17β-estradiol (E2), progesterone, or vehicle as indicated. After another 24 h, the cells were harvested for CAT assay or Luciferase assay. The CAT activity was visualized by Phosphorlmager (Molecular Dynamics) and quantitated by IMAGEQUANT software (Molecular Dynamics). The luciferase activity was determined by the Luminometer (Turner Designs).

(3) Stable Transfection of MCF-7 Cells

The transfection plasmid was created by inserting the SpeI-NotI fragment of pGEM-T EASY-TR4 containing the full length TR4, into the multiple cloning site of autoregulated bidirectional tetracycline-responsive pBIG2i expression vector (Strathdee, C. et al. (1999) Gene 229(1-2) 21-9). MCF-7 cells were transfected with pBIG-2i-TR4 (MCF-7-TR4) or vector (MCF-7-pBIG) using the Superfect reagent (Qiagen) and the cells were maintained in DMEM-10% FBS selective media containing 200 μg/ml hygromycin (GIBCO) for two weeks. Surviving cells were seeded onto 96-well cell culture plates. Cells were grown in selective media for an additional two weeks and then individual colonies were picked and expanded in DMEM 10% FBS.

(4) Glutathione-Sepharose-Transferase (GST) Pull-down Assay

A GST pull-down assay was performed according to the methods described previously (Lee, Y. et al. (1999) Proc. Natl. Acad Sci. USA. 96(26) 14724-9).

Briefly, the GST fusion protein and GST control protein were expressed in an Escherichia coli strain BL21 (DE3) pLys bacterial culture and recovered on glutathione-Sepharose-4B beads. Equal amounts of GST fusion protein bound to glutathione-Sepharose-4B beads were incubated for 2 h at 4° C. with 5 μl of in vitro translated [³⁵S]methionine-labeled protein in a total volume of 100 pi of incubation buffer (20 mM HEPES/pH 7.9, 150 mM KCl, 5 mM MgCl₂, 0.5 mM EDTA, 0.5 mM dithiothreitol, 0.1% Nonidet P-40, 1 mg/ml bovine serum albumin, and 10% glycerol), and protease inhibitor mixture (1 mM phenylmethylsulfonyl fluoride, 1 μg/ml aprotinin, leupeptin, and pepstatin). The beads were then washed three times with wash buffer (20 mM Tris/pH 8.0, 100 mM NaCl, 1 mM EDTA, and 0.5% Nonidet P-40), boiled in 2×SDS sample buffer, loaded onto SDS-polyacrylamide gels, and visualized by autoradiography.

(5) Northern Blotting Analysis

MCF-7-TR4 or MCF-7-pBIG cells were cultured in DMEM containing 10% charcoal dextran-treated FBS for 3 days and then 3×10⁶ cells were seeded on 100-mm dishes. After 24 h, the cells were treated with 2 μg/ml doxycycline (Dox), a derivative of tetracycline, for 24 h and then treated for 48 h with 100 nM E2 or vehicle. Total RNA from cells was prepared by the ultracentrifugation method as described previously (Lee, Y. et al. (1998) J. Biol. Chem. 273(22) 13437-43).

A probe covering the N-terminal of TR4 was released by EcoRI and AatII digestion and labeled with [³²P]dCTP using a random primer DNA labeling system (Amersham Pharmacia Biotech). RNA samples (30 μg) were electrophoresed and transferred onto a nylon membrane. The blot was hybridized with the human TR4 and β-actin sequentially and analyzed by autoradiography.

(6) Western Blotting Analysis

MCF-7-TR4 or MCF-7-pBIG cells were cultured in DMEM containing 10% charcoal dextran-treated FBS for 3 days and then 1×10⁶ cells were seeded on 100-mm dishes. After 24 h, the cells were treated with 2 μg/ml Dox for 24 h and then treated with 100 nM E2 or vehicle. Cell lysates were collected after 12 h with or without E2 treatment by using RIPA buffer. The lysate protein amount was quantitated by Bradford assay (Bio-Rad, Hercules, Calif.) with bovine serum albumin as a reference standard. One hundred μg protein was loaded and after electrophoresis on 10% SDS-PAGE was transferred onto Immobulin (Milipore, Bedford, Mass.). The membrane was blotted with anti-cyclin D1 rabbit polyclonal Ab (H-295) (Santa Cruz). Alkaline phosphatase conjugated secondary Ab was used.

(7) Electrophoretic Mobility Shift Assay

Electrophoretic Mobility Shift Assay was carried out as described previously (Lee, Y. et al. (1998) J Biol. Chem. 273(22) 13437-43). Briefly, 2.5 μl of TNT-expressed ER with different amounts of TR4 was included in each reaction and TNT lysate was used to make up equal amounts of lysate. The reaction was preincubated for 15 min at room temperature in 20 μl of binding buffer (10 mM HEPES/pH 7.4, 50 mM KCl, 1 mM MgCl₂, 1 mM mercaptoethanol, 0.1 mM ZnCl₂, and 20% glycerol) containing 1 μg poly(dI-dC). [³²P]ATP end labeled consensus ERE probes were added to the samples, incubated for 30 min at room temperature, and followed by another 30 min at 4 C. For antibody supershift analyses, the reactions were incubated with 2 μl of a monoclonal anti-ER antibody (C-314) (Santa Cruz) for 15 min at room temperature prior to the addition of probe. Protein-DNA complexes were resolved on a 5% native polyacrylamide gel and analyzed by autoradiography.

(8) MCF-7 Cells Growth Assay

MCF-7-TR4 and MCF-7-pBIG cells, which were deprived of estrogen by culturing in phenol-free DMEM medium supplement with 10% charcol-stripped FBS for 4 days, were plated at 10⁴ cells/well into 24-well plate. After 24 hr Dox treatment, 10 nM E2 was added to cells. Cells were trypsinized at the specified time points and counted with a hemocytometer to determine cell density per sample.

b) Results

(1) TR4 Inhibits the Transactivation of ERs in Mammalian Cells

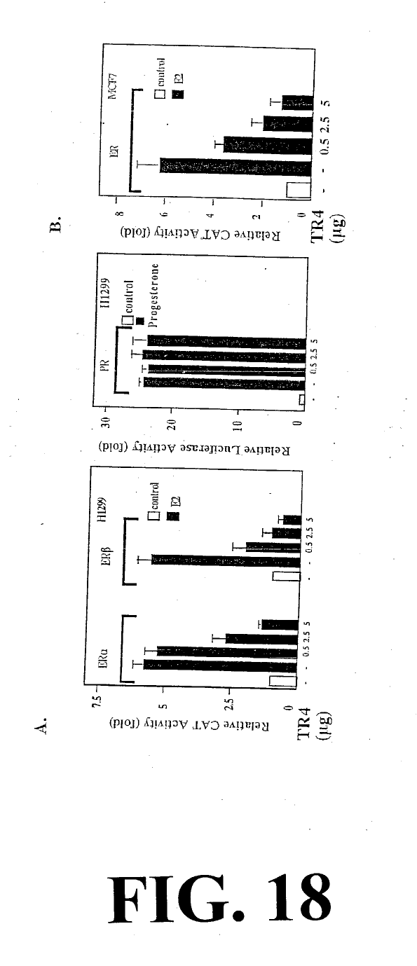

In order to determine the effect of TR4 on the modulation of ER-mediated transactivation, a human lung cancer H1299 cells were transfected with either ERα or ERβ expression plasmid and cotransfected with increasing amounts of TR4 expression plasmid to monitor the transactivation with an ERE-CAT reporter. As shown in FIG. 18A, with exogenous ERs, these cells became responsive to E2 by induction of ERE-CAT reporter activity in the presence of 10 nM E2. Co-transfection of wild type TR4 with either ERα or ERβ resulted in the suppression of the E2-induced CAT activity in a dose-dependent manner. As a control, progesterone receptor (PR), which like ER also plays important roles in the mammary gland development (Lydon, J. et al. (1995) Genes Dev. 9(18) 2266-78), was applied to demonstrate that TR4 has selective suppression with these two closely related nuclear receptors. As shown in FIG. 18A, while 10 nM progesterone can induce PR-mediated MMTV-LUC reporter activity, TR4 failed to suppress the transactivation of PR. The distinct difference in suppression of ER-mediated and PR-mediated transactivation suggests that these events are rather selective. This difference is also not an artifact due to a large amount of exogenously transfected TR4 plasmid, which can result in suppression of general gene transactivation. To further rule out the problem of potential artifacts from exogenously transfected ER, TR4's ability to repress the MCF-7 endogenous ER-mediated transactivation was also assayed. As shown in FIG. 18B, increasing the TR4 led to a gradual decrease in the endogenous ER-mediated CAT reporter activity. Together, data in FIG. 18 suggest TR4 is able to repress ER-, but not PR-mediated transactivation.

(2) Interaction between TR4 and ER

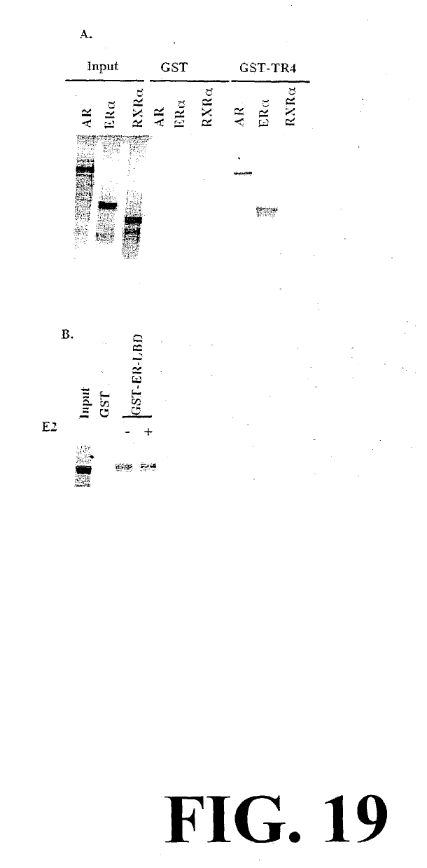

To dissect the mechanism of how TR4 can repress ER transactivation; a GST pull-down assay was used. As shown in FIG. 19A, in the absence of E2, [³⁵S]-methionine labeled ER was able to interact with the GST-TR4 fusion protein but not with GST alone. This interaction is relatively specific for ER, as TR4 was unable to interact with RXRα, a common nuclear receptor that binds to many other nuclear receptors (Mangelsdorf, D. et al. (1995) Cell 83(6) 841-50). Results from FIG. 19A are consistent with an earlier report showing TR4 fails to bind to RXR in mammalian two-hybrid assay (Yan, Z. et al. (1998) J. Biol. Chem. 273(18) 10948-57). For the positive control, FIG. 19A also demonstrates that TR4 binds well to AR. FIG. 19B further demonstrates that LBD of ER interacts well with TR4 in the presence or absence of E2. In summary, GST pull-down assays demonstrate that TR4 interacts with ER in the presence or absence of E2.

(3) TR4-LBD Domain Is Essential for TR4 Suppression Effect on ER Transactivation

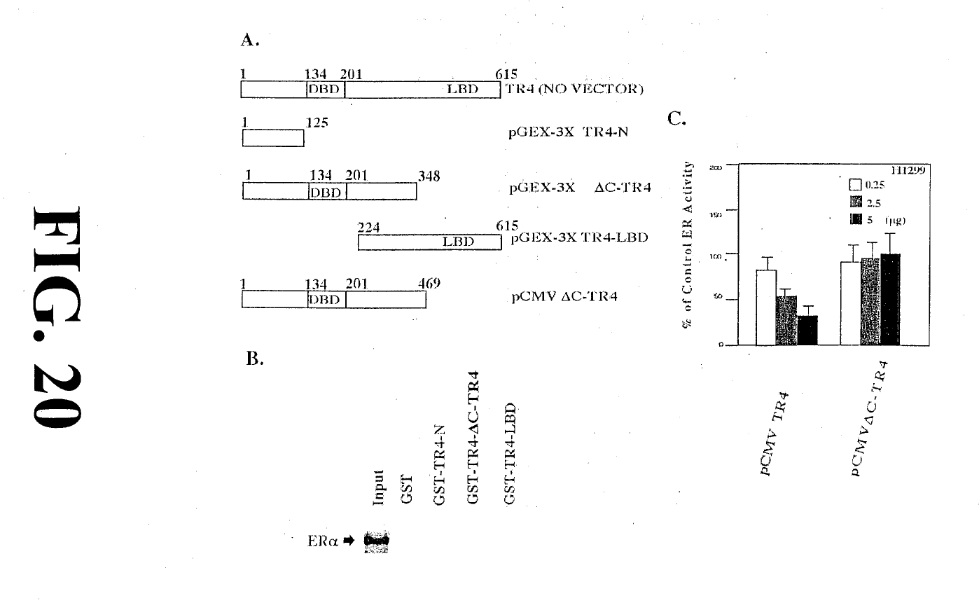

To dissect which domain within the TR4 can bind to ER, GST-TR4 N-terminal, GST-ΔC-TR4, and GST-TR4-LBD fusion proteins were generated (FIG. 20A) and their interaction with ER was tested. As shown in FIG. 20B, only GST-TR4-LBD was able to interact with [³⁵S]-ER, but not GST-TR4 N-terminal and GST-AC-TR4. An expression vector with a TR4 mutant lacking the C-terminal of LBD (PCMV ΔC-TR4) (FIG. 20A) to determine if this mutant can still repress ER transactivation was constructed. As shown in FIG. 20C, while full-length TR4 could repress ER transactivation in a dose-dependent manner, the pCMV ΔC-TR4 showed no repression on the ER transactivation. Together, data from FIG. 20 demonstrate that the suppression effect of TR4 on ER relies on the physical interaction between two proteins and the LBD within the TR4 C-terminal is an essential domain for TR4 to interact and repress ER transactivation.

(4) Interruption of ER-ERE Binding and ER homodimerization Via LBD by TR4

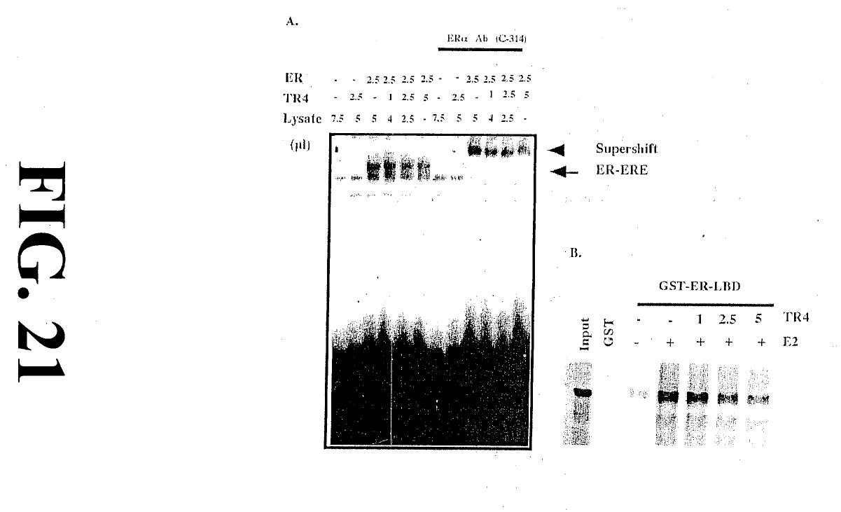

To further dissect the molecular mechanism of how TR4 can repress ER target gene expression, EMSA was used to investigate whether the inhibitory effect of TR4 is exerted at the level of ER-ERE binding. FIG. 21A, using in vitro expressed TR4 protein from rabbit reticulate lysate TNT system in gel-shift assay, demonstrates that the ER-[³²P]ERE binding band (arrow) could be reduced by adding increasing amounts of TR4. TR4 could also reduce the amount of the ER-ERE supershifted complex in the presence of an ERα antibody (arrowhead). This finding clearly demonstrates that direct interaction between TR4 and ER will lead to interruption or prevention of the ER binding to ERE. To further test other possible mechanisms, the influence of TR4 on ER binding with its coregulators or itself (homodimerization) was assayed. Results in FIG. 21B demonstrate that TR4 can also inhibit ER-ER homodimerization. In contrast, TR4 shows little influence on the binding between ER and its coregulator, RIP140 (Cavailles, V. et al. (1995) Embo. J. 14(15) 3741-51).

(5) Suppression of ER Target Gene Expression in MCF-7 cells with Tetracycline-Induced TR4

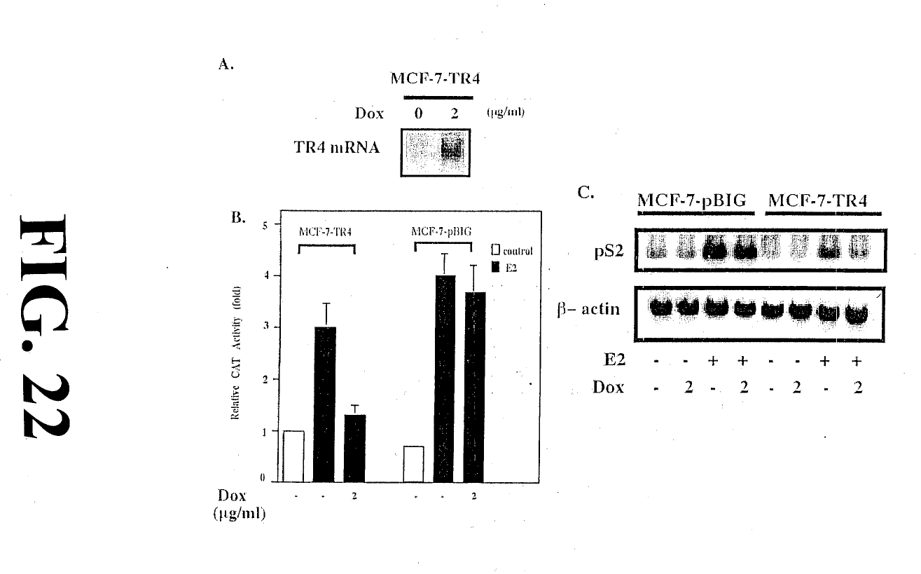

To rule out potential artifacts with these transient transfection methods, a tetracycline-induced TR4 expressed MCF-7 cell line (MCF-7-TR4) was constructed. As shown in FIG. 22A, addition of 2 μg/ml Dox induces TR4 expression in MCF-7-TR4 cells. The induction of TR4 could then repress the ERE-CAT activity. In contrast, addition of 2 μg/ml Dox in control MCF-7-pBIG cells, (cells stably transfected with parent vector pBIG), showed little or no influence on the ERE-CAT (FIG. 22B). FIG. 22C further demonstrated that Dox-induced TR4 could also repress the ER endogenous target gene pS2 (Brown, A. et al. (1984) Proc. Natl. Acad. Sci. USA. 81(20), 6344-8) expression, but had no influence on the β-actin gene expression, which serves as a negative control. Together, FIG. 22 clearly demonstrates that TR4 can repress ER target gene expression in the MCF-7 cells stably transfected with TR4.

(6) TR4 Expression in MCF-7 Inhibits the Estrogen-Stimulated Cell Growth and Cyclin D1 Expression

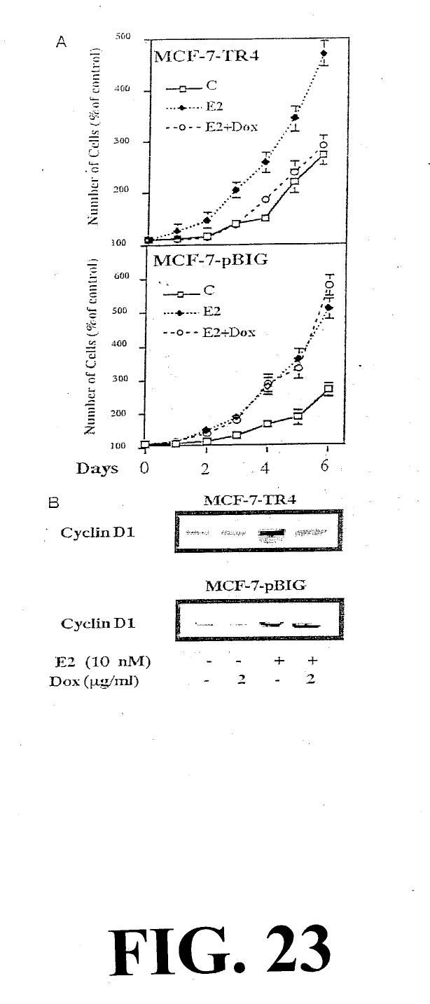

We then studied the potential physiological consequence of the TR4 suppression of ER transactivation. As it is well documented that E2/ER play pivotal roles for the breast cancer growth (Soule, H. et al. (1980) Cancer Lett. 10(2), 177-89; Henderson, B. et al. (1988) Cancer Res. 48(2), 246-53), whether TR4 can also modulate E2/ER-mediated breast cancer MCF-7 cell growth was determined. As shown in FIG. 23A, addition of 10 nM E2 stimulated cell growth in both MCF-7-TR4 and MCF-7-pBIG cells. Addition of Dox to both cells lines, however, only repressed cell growth in the MCF-7-TR4 cells, suggesting that Dox-induced TR4 could repress the E2/ER-mediated cell growth. To further dissect the potential mechanism of how TR4 repressed the E2/ER-mediated cell growth, the expression of cyclin D1, a cell cycle regulator responsible for Gl-S phase transition, which has been linked to the E2/ER-mediated cell growth (Prall, O. et al. (1998) J. Steroid Biochem. Mol. Biol. 65(1-6), 169-74) was examined.

As shown in FIG. 23B, in MCF-7-TR4 cells, addition of 10 nM E2 induced cyclin D1 expression and this induction was repressed by adding Dox. In contrast, in MCF-7-pBIG cells there was no influence on the E2-induced cyclin D1 gene expression after adding Dox. Together, FIG. 23 suggests that TR4 can be able to repress E2/ER-mediated cell growth via modulation of the ER target genes such as cyclin D1 expression.

Early studies in nuclear receptors suggested that TR, RAR, SHP, and chicken ovalbumin upstream promoter-transcriptional factor, can be able to interact with ER and modulate ER transactivation (Lee, S. et al. (1998) Mol. Endocrinol. 12(8):1184-92; Seol, W. et al. (1998) Mol. Endocrinol. 12(10) 1551-7; Johansson, L. et al. (1999) J. Biol. Chem. 274(1) 345-53; Klinge, C. et al. (1997) J. Biol. Chem. 272(50), 31465-74. 483. As these receptors, together with their ligands such as T₃ or retinoids, can play important roles in the ER-mediated cell growth (Fontana, J. A. (1987) Exp. Cell Biol. 55(3): 136-44; Fontana, J. et al. (1990) Cancer Res. 50(7), 1977-82), it is consistent that the pleiotropic effect of estrogen can require the cooperation of ER with a large network of nuclear receptors. Disclosed herein, TR4 is identified as one of the ER-interacting proteins to modulate ER functions.

This competition mechanism is, therefore, different from the TR4 suppression of ER transactivation, which is through protein-protein interaction (FIG. 19). The fact that there is no extra band in the EMSA when TR4 was added alone or along with ER and incubated with [³²P]ERE (FIG. 21A), clearly suggests that TR4 will not bind to [³²P]ERE. Instead, the TR4 will reduce ER-[³²P]ERE binding through heterodimer formation with ER, which therefore titrates out the free ER. In addition to the interruption of binding between ERE and ER, the data also demonstrated that TR4 could inhibit ER-ER homodimerization (FIG. 21B). FIG. 20 further demonstrates that TR4 can interact with ERα via its LBD. Both ER and TR4 can use their LBDs to interact with other receptors. The disclosed data show that TR4 did not influence the binding between RIP 140 and ER. Instead, TR4 modulates ER functions via interruption of ER homodimerization and ER binding to EREs.

Furthermore, the TR4 stably transfected MCF-7 cells clearly demonstrate that TR4 could inhibit the expression of estrogen-induced pS2 mRNAs (Brown, A. et al. (1984) Proc. Natl. Acad. Sci. USA. 81(20), 6344-8), which is specifically and directly induced by estrogens through ER at the transcriptional level in MCF-7 cells (FIG. 22C). Since the expression of pS² gene has been widely used as a marker to monitor the effect of estrogens, this result suggests that TR4 could suppress ER function not only occurring in cell line transient transfection experiments (FIGS. 18A, B), but also in ER target gene expression. TR4 is also able to suppress estrogen-induced cell proliferation in MCF-7 cells stably transfected with TR4 induced by Dox (FIG. 22A). This result extends the in vitro results and demonstrates that the consequence of the protein-protein inhibition can result in the suppression of E2/ER-induced cell growth.

Estrogen induced breast cancer cell proliferation has been linked well to the modulation of cyclin D1 that plays important roles in the cell cycle control to stimulate the G1-S phase progression Prall, O. et al. (1998) J. Steroid Biochem. Mol. Biol. 65(1-6), 169-74; Baldin, V. et al. (1993) Genes Dev. 7(5), 812-21).

Results from FIG. 23B also demonstrate that TR4 could suppress E2/ER-regulated cyclin D1 gene expression. This Dox-inducible cell model system to control expression of TR4 and the function of ER provides a system for studying the physiological roles of TR4 in ER target organs, such as breast and testis.

Any compounds or small peptide(s) that mimic the interaction domain between TR4 and ER could be developed for future therapeutic uses.

Tissue distribution studies indicated that ERα is expressed well in testes and epididymis (Couse, J. et al. (1997) Endocrinology 138(11), 4613-21). ERαKO mice are infertile and produce lower numbers of epididymal sperm, compared to wild-type mice at 12 weeks. Furthermore, the sperms produced in the ERαKO mice have obvious defects and are unable to fertilize wild-type oocytes (Eddy, E. et al. (1996) Endocrinology 137(11), 4796-805). These studies indicated that ERα plays important roles in the testes function and spermatogenesis.

TR4 is highly expressed in testes, and is also strongly linked to the defective spermatogenesis found in rhesus monkey in either surgery-induced cryptorchid testis (Mu, X. et al. (2000) J. Biol. Chem. 275(31), 23877-83).

### 4. Example 4

### TR2 Orphan Receptor Functions as a Negative Modulator for Androgen Receptor in Prostate Cancer Cells PC-3

Both androgen receptor (AR) and orphan receptor TR2 (TR2) belong to the steroid nuclear receptor superfamily and all express in prostate cancer tissue and cell lines. AR has been known to be involved in prostate proliferation and prostate cancer progression, and AR binds to androgen response elements and regulates target gene expression via a mechanism involving coregulators. Transient transfection and CAT reporter gene assays were employed to assess AR-mediated transactivation. AR target gene prostate specific antigen (PSA) expression level was measured by Northern blot analysis. The interaction between AR and TR2 was assessed by Glutathione-S-Transferase (GST) Pull-down assay. Orphan nuclear receptor TR2 suppressed androgen-mediated transactivation in prostate cancer PC-3 cells. Over-expression of TR2 suppressed AR target gene PSA expression. The suppression of AR mediated transactivation by TR2 is not due to competition for the limited coregulator availability by these two receptors, and is consistent with the interaction between TR2 and AR nuclear receptors. The results indicate that TR2 functions as a negative modulator in prostate cancer.

a) Materials and methods

(1) Plasmids

The plasmid MMTV-CAT, PSA-CAT, pCMV-AR, pCMV-TR2, pSG5-PR, pSG5-GR, pSG5-SRC-1, pSG5-TIFII, pSG5-ARA55, pSG5-ARA54, GST-TR2, and pCMV-β-Gel were reported previously [5-13F, 16F, and their sequences and structures are herein incorporated by reference].

(2) Transient Transfection and CAT Assay

Human prostate cancer PC-3 cells were maintained in DMEM containing penicillin (25 units/ml), streptomycin (25 g/ml), and 5% FCS. Human prostate cancer LNCaP cells were maintained in RPMI 1640 containing penicillin (25 units/ml), streptomycin (25 g/ml), and 10% FCS. PC-3 cell were transfected using the calcium phosphate precipitation method as described previously [Mu X. M. et al. (1998) Endocrine 9:27-32], and LNCaP cells were transfected by using Superfect™ according to the manufacturer's procedures (Qiagen, Chatsworth, Calif.). CAT assay was performed as described previously [Lee Y. et al. (1999) Proc Natl Acad Sci USA 96:14724-14729], and is herein incorporated by reference at least for material related to the CAT assays].

(3) Glutathione-S-Transferase (GST) Pull-Down Assay

GST control protein and GST-TR2 fusion protein were purified by glutathione-sepharose 4B beads as described by manufacture (Amersham. Biosciences). Five microliters of in vitro-translated 35S-methionine-labeled proteins was used to perform the Pull-down assay as described previously [4F].

(4) Northern Blot

Total RNA from transfected LNCaP cells was prepared using TRIZOL reagents (Life Technology) as instructed by manufacturer. The probe was obtained from the PSA gene by PCR and labeled with 32P dCTP. Northern hybridization was performed as described previously [Mu X. M. et al. (2002) J Biol Chem 275:23877-23883].

b) Results

(1) TR2 Suppresses AR Mediated Transactivation in Prostate Cancer PC-3 cells

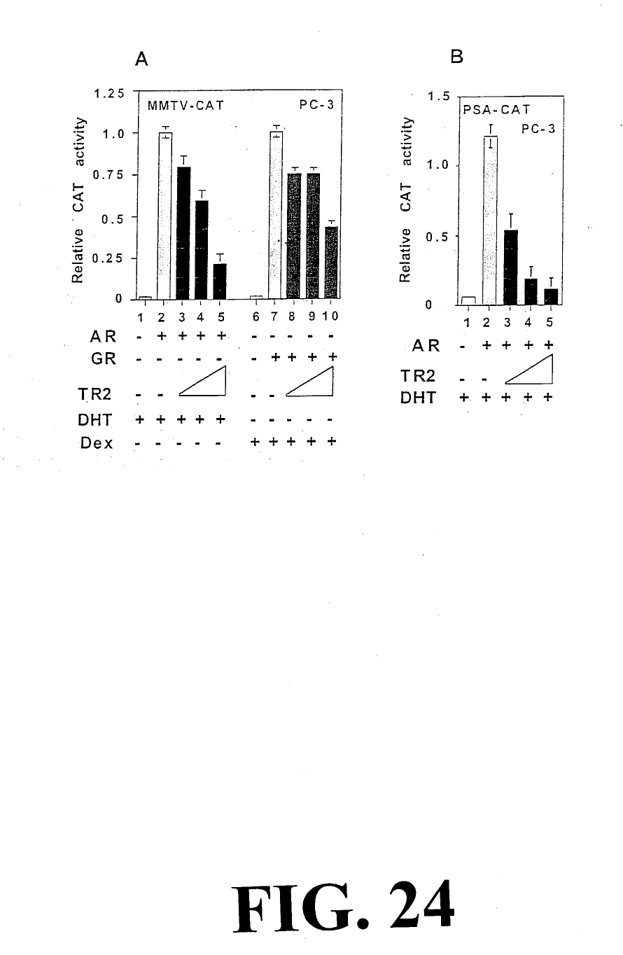

AR is known to be highly involved in prostate cancer progression, and both TR2 and AR are expressed in prostate cancer, therefore the TR2 effect on AR transactivation activity was analyzed. As shown in FIG. 24, addition of AR induced both MMTV-CAT (A) and PSA-CAT (B), two common AR target gene reporters in prostate cancer PC-3 cells, activity in the presence of 10 nM DHT. Addition of TR2 expression plasmid strongly suppresses MMTV-CAT (FIG. 24A) and PSA-CAT (FIG. 24B) activity (Lanes 3-5 vs 2) in a dosage-dependent manner. Glucocorticoid receptor (GR) can also induce MMTV reporter gene activity in the presence of 10 nM dexamethasone (Lane 7 vs 6), but transfection of TR2 has less suppression effect on the GR-mediated transactivation (Lanes 8-10 vs 7).

(2) Suppression of AR-Mediated Transactivation is Not Due to the Competition of Limited Coregulator Availability Between AR and TR2

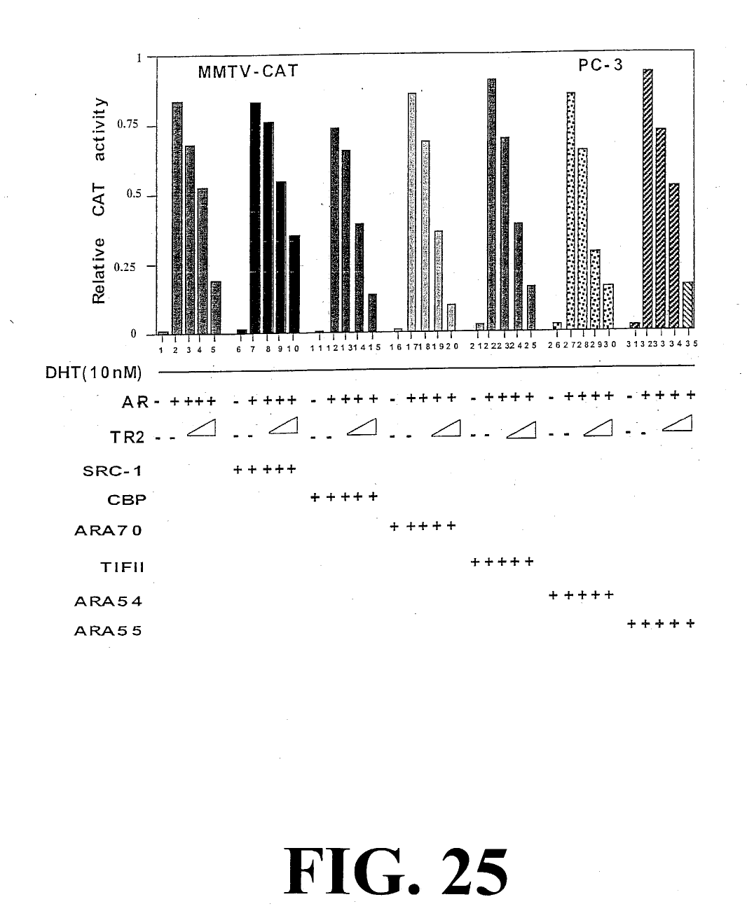

It has been known that AR transactivation activity is highly regulated by coregulators, and the amount of co-regulator is relatively limited, therefore nuclear receptors can be able to suppress each other via competing for shared coregulators. As shown in FIG. 25, to demonstrate whether the suppression of AR transactivation by TR2 is due to competing for the limited coregulators, MMTV-CAT, pCMV-AR, and increasing amount of pCMV-TR2 were transfected into human prostate PC-3 cells, and one or more of the AR coregulators SRC-1 (Lane 6-10), CBP (Lane 11-15), ARA70 (Lane 16-20), TIF II (Lane 21-25), ARA54 (Lane 26-30), and ARA55 (Lane 31-35) were also transfected. Co-transfection of these coregulators did not affect the suppression of AR mediated-transactivation by TR2. This data suggested that suppression of AR-mediated transactivation was not due to the competition of limited coregulator availability between AR and TR2 and the suppression of AR-mediated transactivation by TR2 is a specific event in PC-3 cells.

(3) TR2 Suppresses AR Target Gene PSA Expression

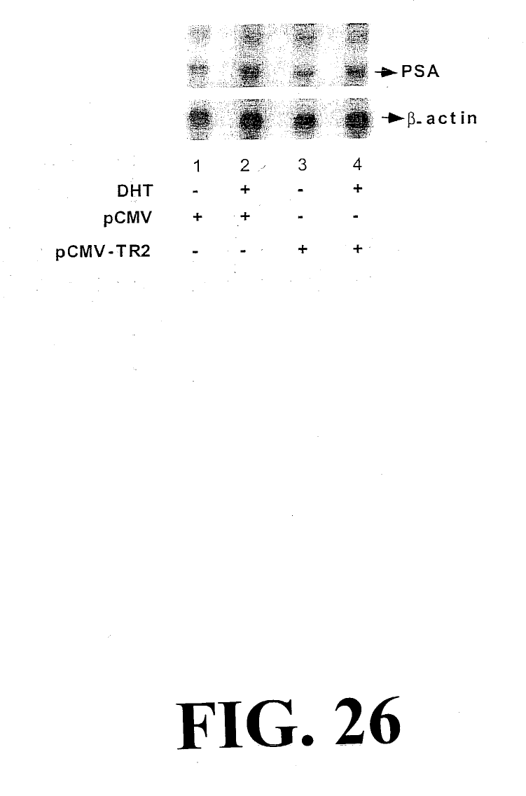

PSA is an androgen target gene, which is widely used as a marker for prostate cancer progression. Prostate cancer LNCaP cells were transfected with either vector control or TR2 expression plasmid pCMV-TR2, and cells were treated with or without DHT. Total RNA was extracted and PSA expression was measured by Northern blot. As shown in FIG. 26, addition of 10 nM DHT induces PSA mRNA expression (Lane 2 vs 1). Addition of TR2 expression plasmid suppresses endogenous PSA expression level in LNCaP cells (Lane 4 via 2). These in vivo TR2-mediated suppressive effects strongly support the data from reporter gene assays shown in FIG. 24, and demonstrate that TR2 can function as a repressor to negatively regulate PSA expression.

(4) Interaction Between TR2 and AR

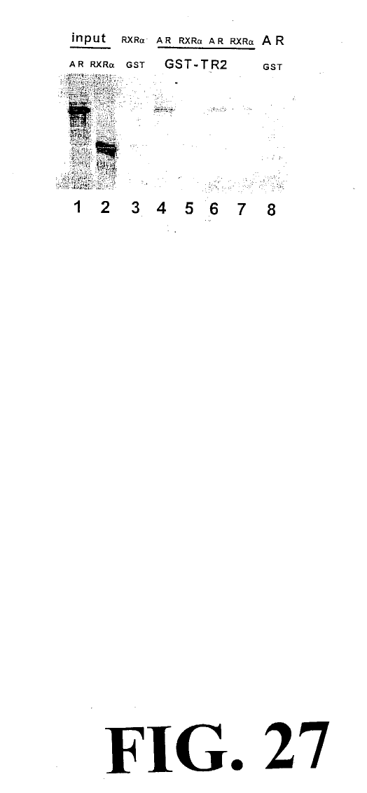

To further dissect the mechanism of how TR2 can suppress AR-mediated transactivation, a GST Pull-down assay was applied. As shown in FIG. 27, 35S methionine-labeled AR was able to interact with GST-TR2 fusion protein (Lane 4 and 6), but not GST alone (Lane 3 and 8). This interaction was relatively specific for AR, as TR2 was not able to interact with RXRα (Lane 5 and 7), a common nuclear receptor that binds to many other nuclear receptors.

Early studies showed that TR2 is a transcription factor that can regulate several target genes expression [5F-11F]. Disclosed herein, TR2 can also function as a negative modulator for AR transactivation in prostate cancer cells. Both AR and TR2 are expressed in prostate and prostate cancer. The cross-talk between these two nuclear receptors is consistent with a new regulatory mechanism in prostate cells and prostate cancer cells.

Dimerization is essential for most nuclear receptor functions. RAR T3R, vitamin D receptor, orphan receptors LXR and FXR, all form heterodimers with the common partner RXR [Mangelsdorf D. J. et al. (1995) Cell 83:841-850]. Recently the interaction and mutual suppression between AR and orphan receptor TR4, a closely related orphan receptor to TR2, have been reported [Lee Y. et al. (1999) Proc Natl Acad Sci USA 96:14724-14729]. Suppression of estrogen receptor (ER)-mediated transactivation by TR2 and TR4 has also been reported [4F, 20F]. Homodimerization of TR2 and TR4 and heterodimerization between TR2 and TR4 have also been reported [21F].

G. Sequences

| 1. SEQ ID NO:1 Human TR2 Protein Genbank accession number P13056. | |
|---|---|
| 1 | matieeiahq iieqqmgeiv teqqtgqkiq ivtaldhntq gkqfiltnhd gstpskvila |
| 61 | rqdstpgkvf lttpdaagvn qlffttpdls aqhlqlltdn spdqgpnkvf dlcvvcgdka |
| 121 | sgrhygavtc egckgffkrs irknlvyscr gskdciinkh hrnrcqycrl qrciafgmkq |
| 181 | dsvqcerkpi evsrekssnc aastekiyir kdlrspltat ptfvtdsest rstglldsgm |
| 241 | fmnihpsgvk tesavlmtsd kaescqgdls tlanvvtsla nlgktkdlsq nsnemsmies |
| 301 | lsnddtslce fqemqtngdv srafdtlaka lnpgestacq ssvagmegsv hlitgdssin |
| 361 | ytekegplls dshvafrltm pspmpeylnv hyigesasri lflsmhwals ipsfqalgqe |
| 421 | nsislvkayw nelftlglaq cwqvmnvati latfvnclhn slqqdakvia alihftrrai |
| 481 | tdl |
| 2. SEQ ID NO2 Genbank Accession no. A36738. orphan receptor TR2, splice | |
| form TR2-9-human. | |
| 1 | matieeiahq iieqqmgeiv teqqtgqkiq ivtaldhntq gkqfiltnhd gstpskvila |
| 61 | rqdstpgkvf lttpdaagvn qlffttpdls aqhlqlltdn spdqgpnkvf dlcvvcgdka |
| 121 | sgrhygavtc egckgffkrs irknlvyscr gskdciinkh hrnrcqycrl qrciafgmkq |
| 181 | dsvqcerkpi evsrekssnc aastekiyir kdlrspltat ptfvtdsest rstglldsgm |
| 241 | fmnihpsgvk tesavlmtsd kaescqgdls tlanvvtsla nlgktkdlsq nsnemsmies |
| 301 | lsnddtslce fqemqtngdv srafdtlaka lnpgestacq ssvagmegsv hlitgdssin |
| 361 | ytekegplls dshvafrltm pspmpeylnv hyigesasrl lflsmhwals ipsfqalgqe |
| 421 | nsislvkayw nelftlglaq cwqvmnvati latfvnclhn slqqaeg |
| 3. SEQ ID NO:3 Genbank Accession No. B36738 orphan receptor TR2, splice | |
| form TR2-11-human. | |
| 1 | matieeiahq iieqqmgeiv teqqtgqkiq ivtaldhntq gkqfiltnhd gstpskvila |
| 61 | rqdstpgkvf lttpdaagvn qlffttpdls aqhlqlltdn spdqgpnkvf dlcvvcgdka |
| 121 | sgrhygavtc egckgffkrs irknlvyscr gskdciinkh hrnrcqycrl qrciafgmkq |
| 181 | dsvqcerkpi evsrekssnc aastekiyir kdlrspltat ptfvtdsest rstglldsgm |
| 241 | fmnihpsgvk tesavlmtsd kaescqgdls tlanvvtsla nlgktkdlsq nsnemsmies |
| 301 | lsnddtslce fqemqtngdv srafdtlaka lnpgestacq ssvagmegsv hlitgdssin |
| 361 | ytekegplls dshvafrltm pspmpeylnv hyigesasrl lflsmhwals ipsfqalgqe |
| 421 | nsislvkayw nelftlglaq cwqvmnvati latfvnclhn slqqdkmste rrkllmehif |
| 481 | klqefcnsmv klcidgyeya ylkaivlfsp dhpslenmel iekfqekayv efqdyitkty |
| 541 | pddtyrlsrl llrlpalrlm natiteelff kglignirid sviphilkme padynsqiig |
| 601 | hsi |
| 4. SEQ ID NO:4 Genbank Accession No. A31521. orphan receptor TR2, splice | |
| form TR2-5-human. | |
| 1 | matieeiahq iieqqmgeiv teqqtgqkiq ivtaldhntq gkqfiltnhd gstpskvila |
| 61 | rqdstpgkvf lttpdaagvn qlffttpdls aqhlqlltdn spdqgpnkvf dlcvvcgdka |
| 121 | sgrhygavtc egckgffkrs irknlvyscr gskdciinkh hrnrcqycrl qrciafgmkq |
| 181 | dsvqcerkpi evsrekssnc aastekiyir kdlrspltat ptfvtdsest rstglldsgm |
| 241 | fmnihpsgvk tesavlmtsd kaescqgdls tlanvvtsla nlgktkdlsq nsnemsmies |
| 301 | lsnddtslce fqemqtngdv srafdtlaka lnpgestacq ssvagmegsv hlitgdssin |
| 361 | ytekegplls dshvafrltm pspmpeylnv hyigesasrl lflsmhwals ipsfqalgqe |
| 421 | nsislvkayw nelftlglaq cwqvmnvati latfvnclhn slqqdakvia alihftrrai |
| 481 | tdl |
| 5. SEQ ID NO:5 Genbank Accession No. 154075. gene mTR2R1 protein-mouse | |
| 1 | matnmeglvq hrvgtqqvae vprtqtswpe spgmtspspr iqiistdsav aspqriqivt |
| 61 | dqqtgqkiqi vtavdasgss kqqfiltspd gagtgkvila spetssakql ifttsdnlvp |
| 121 | griqivtdsa sverllgkad vqrpqvveyc vvcgdkasgr hygavscegc kgffkrsvrk |
| 181 | nltyscrssq dciinkhhrn rcqfcrlkkc lemgmkmesv qserkpfdvq rekpsncaas |
| 241 | tekiyirkdl rspliatptf vadkdgarqt glldpgmlvn iqqpliredg tvllaadska |
| 301 | etsqgalgtl anvvtslanl seslnngdas emqpedqsas eitrafdtla kalnttdsas |
| 361 | ppsladgida sgggsihvis rdqstpiiev egpllsdthv tfkltmpspm peylnvhyic |
| 421 | esasrllfls mhwarsipaf qalgqdcnts lvracwnelf tlglaqcaqv mslstilaai |
| 481 | vnhlqnsiqe dklsgdrikq vmehiwklqe fcnsmakldi dgyeyaylka ivlfspdhpg |
| 541 | ltgtsqiekf qekaqmelqd yvqktysedt yrlarilvrl palrlmssni teelfftgli |
| 601 | gnvsidsiip yilkmetaey ngqitgasl |
| 6. :SEQ ID NO:6 Genbank Accession No. M29959. Human steroid receptor | |
| (TR2-9) Protein. | |
| MATIEEIAHQIIEQQMGEIVTEQQTGQKIQIVTALDHNTQGKQF | |
| ILTNHDGSTPSKVILARQDSTPGKVFLTTPDAAGVNQLFFTTPDLSAQHLQLLTDNSP | |
| DQGPNKVFDLCVVCGDKASGRHYGAVTCEGCKGFFKRSIRKNLVYSCRGSKDCIINKH | |
| HRNRCQYCRLQRCIAFGMKQDSVQCERKPIEVSREKSSNCAASTEKIYIRKDLRSPLT | |
| ATPTFVTDSESTRSTGLLDSGMFMNIHPSGVKTESAVLMTSDKAESCQGDLSTLANVV | |
| TSLANLGKTKDLSQNSNEMSMIESLSNDDTSLCEFQEMQTNGDVSRAFDTLAKALNPG | |
| ESTACQSSVAGMEGSVHLITGDSSINYTEKEGPLLSDSHVAFRLTMPSPMPEYLNVHY | |
| IGESASRLLFLSMHWALSIPSFQALGQENSISLVKAYWNELFTLGLAQCWQVMNVATI | |
| LATFVNCLHNSLQQAEG″ | |
| 7. SEQ ID NO:7 Genbank Accession No. M29959. Human steroid receptor | |
| (TR2-9) mRNA, complete cds. Encodes protein designated M29959 herein. | |
| 1 | gaattcgggc ccgtcggctt tcttcaaccc tctcttcccg gagcgccccc aatccacgag |
| 61 | tggcagccgc gggactgtcg cgtcggcgcc cgacgcggag tcagcagggg cgaaaagcgg |
| 121 | tagatcatgg caaccataga agaaattgca catcaaatta ttgaacaaca gatgggagag |
| 181 | attgttacag agcagcaaac tgggcagaaa atccagattg tgacagcact tgatcataat |
| 241 | acccaaggca agcagttcat tctgacaaat cacgacggct ctactccaag caaagtcatt |
| 301 | ctggccaggc aagattccac tccgggaaaa gttttcctta caactccaga tgcagcaggt |
| 361 | gtcaaccagt tattttttac cactcctgat ctgtctgcac aacacctgca gctcctaaca |
| 421 | gataattctc cagaccaagg accaaataag gtttttgatc tttgcgtagt atgtggagac |
| 481 | aaagcatcag gacgtcatta tggagcagta acttgtgaag gctgcaaagg attttttaaa |
| 541 | agaagcatcc gaaaaaattt agtatattca tgtcgaggat caaaggattg tattattaat |
| 601 | aagcaccacc gaaaccgctg tcaatactgc aggttacaga gatgtattgc gtttggaatg |
| 661 | aagcaagact ctgtccaatg tgaaagaaaa cccattgaag tatcacgaga aaaatcttcc |
| 721 | aactgtgccg cttcaacaga aaaaatctat atccgaaagg accttcgtag cccattaact |
| 781 | gcaactccaa cttttgtaac agatagtgaa agtacaaggt caacaggact gttagattca |
| 841 | ggaatgttca tgaatattca tccatctgga gtaaaaactg agtcagctgt gctgatgaca |
| 901 | tcagataagg ctgaatcatg tcagggagat ttaagtacat tggccaatgt ggttacatca |
| 961 | ttagcgaatc ttggaaaaac taaagatctt tctcaaaata gtaatgaaat gtctatgatt |
| 1021 | gaaagcttaa gcaatgatga tacctctttg tgtgaatttc aagaaatgca gaccaacggt |
| 1081 | gatgtttcaa gggcatttga cactcttgca aaagcattga atcctggaga gagcacagcc |
| 1141 | tgccagagct cagtagcggg catggaagga agtgtacacc taatcactgg agattcaagc |
| 1201 | ataaattaca ccgaaaaaga ggggccactt ctcagcgatt cacatgtagc tttcaggctc |
| 1261 | accatgcctt ctcctatgcc tgagtacctg aatgtgcact acattgggga gtctgcctcc |
| 1321 | agactgctgt tcttatcaat gcactgggca ctttcgattc cttctttcca ggctctaggg |
| 1381 | caagaaaaca gcatatcact ggtgaaagct tactggaatg aactttttac tcttggtctt |
| 1441 | gcccagtgct ggcaagtgat gaatgtagca actatattag caacatttgt caattgtctt |
| 1501 | cacaatagtc ttcaacaagc agaggggtaa tcaccttaaa atgtcatcaa aaatagatct |
| 1561 | actagaaggc agcatcacat tcccatctta cttatggact cctacccctg gttcatgtct |
| 1621 | tatatgcctg taatggttat aaagcctacc ttcaggaaag ctatggttga ctaattacta |
| 1681 | atggatgggt tttaaacatg tccctctaca ataaattaaa atctttcaat gtttgaatat |
| 1741 | aatgtggagg tgtttacctg agggcctctc tatctccccg aattc |
| 8. SEQ ID NO:8 Genbank Accession No. M29960. Human steroid receptor | |
| (TR2-11) protein | |
| MATIEEIAHQIIEQQMGEIVTEQQTGQKIQIVTALDHNTQGKQF | |
| ILTNHDGSTPSKVILARQDSTPGKVFLTTPDAAGVNQLFFTTPDLSAQHLQLLTDNSP | |
| DQGPNKVFDLCVVCGDKASGRHYGAVTCEGCKGFFKRSIRKNLVYSCRGSKDCIINKH | |
| HRNRCQYCRLQRCIAFGMKQDSVQCERKPIEVSREKSSNCAASTEKIYIRKDLRSPLT | |
| ATPTFVTDSESTRSTGLLDSGMFMNIHPSGVKTESAVLMTSDKAESCQGDLSTLANVV | |
| TSLANLGKTKDLSQNSNEMSMIESLSNDDTSLCEFQEMQTNGDVSRAFDTLAKALNPG | |
| ESTACQSSVAGMEGSVHLITGDSSINYTEKEGPLLSDSHVAFRLTMPSPMPEYLNVHY | |
| IGESASRLLFLSMHWALSIPSFQALGQENSISLVKAYWNELFTLGLAQCWQVMNVATI | |
| LATFVNCLHNSLQQDKMSTERRKLLMEHIFKLQEFCNSMVKLCIDGYEYAYLKAIVLF | |
| SPDHPSLENMELIEKFQEKAYVEFQDYITKTYPDDTYRLSRLLLRLPALRLMNATTTE | |
| ELFFKGLIGNIRIDSVIPHILKMEPADYNSQIIGHSI″ | |
| 9. SEQ ID NO:9 Genbank Accession No. M29960. Human steroid receptor | |
| (TR2-11) mRNA, complete cds encoding protein M299960 disclosed herein | |
| 1 | gggactgtcg cgtcggcgcc cgacgcggag tcagcagggg cgaaaagcgg tagatcatgg |
| 61 | caaccataga agaaattgca catcaaatta ttgaacaaca gatgggagag attgttacag |
| 121 | agcagcaaac tgggcagaaa atccagattg tgacagcact tgatcataat acccaaggca |
| 181 | agcagttcat tctgacaaat cacgacggct ctactccaag caaagtcatt ctggccaggc |
| 241 | aagattccac tccgggaaaa gttttcctta caactccaga tgcagcaggt gtcaaccagt |
| 301 | tattttttac cactcctgat ctgtctgcac aacacctgca gctcctaaca gataattctc |
| 361 | cagaccaagg accaaataag gtttttgatc tttgcgtagt atgtggagac aaagcatcag |
| 421 | gacgtcatta tggagcagta acttgtgaag gctgcaaagg attttttaaa agaagcatcc |
| 481 | gaaaaaattt agtatattca tgtcgaggat caaaggattg tattattaat aagcaccacc |
| 541 | gaaaccgctg tcaatactgc aggttacaga gatgtattgc gtttggaatg aagcaagact |
| 601 | ctgtccaatg tgaaagaaaa cccattgaag tatcacgaga aaaatcttcc aactgtgccg |
| 661 | cttcaacaga aaaaatctat atccgaaagg accttcgtag cccattaact gcaactccaa |
| 721 | cttttgtaac agatagtgaa agtacaaggt caacaggact gttagattca ggaatgttca |
| 781 | tgaatattca tccatctgga gtaaaaactg agtcagctgt gctgatgaca tcagataagg |
| 841 | ctgaatcatg tcagggagat ttaagtacat tggccaatgt ggttacatca ttagcgaatc |
| 901 | ttggaaaaac taaagatctt tctcaaaata gtaatgaaat gtctatgatt gaaagcttaa |
| 961 | gcaatgatga tacctctttg tgtgaatttc aagaaatgca gaccaacggt gatgtttcaa |
| 1021 | gggcatttga cactcttgca aaagcattga atcctggaga gagcacagcc tgccagagct |
| 1081 | cagtagcggg catggaagga agtgtacacc taatcactgg agattcaagc ataaattaca |
| 1141 | ccgaaaaaga ggggccactt ctcagcgatt cacatgtagc tttcaggctc accatgcctt |
| 1201 | ctcctatgcc tgagtacctg aatgtgcact acattgggga gtctgcctcc agactgctgt |
| 1261 | tcttatcaat gcactgggca ctttcgattc cttctttcca ggctctaggg caagaaaaca |
| 1321 | gcatatcact ggtgaaagct tactggaatg aactttttac tcttggtctt gcccagtgct |
| 1381 | ggcaagtgat gaatgtagca actatattag caacatttgt caattgtctt cacaatagtc |
| 1441 | ttcaacaaga taaaatgtca acagaaagaa gaaaattatt gatggagcac atcttcaaac |
| 1501 | tacaggagtt ttgtaacagc atggttaaac tctgcattga tggatacgaa tatgcctacc |
| 1561 | tgaaggcaat agtactcttc agtccagatc atccaagcct agaaaacatg gaactgatag |
| 1621 | agaaatttca ggaaaaggct tatgtggaat tccaagatta tataaccaaa acatatccag |
| 1681 | atgacaccta caggttatcc agactactac tcagattgcc agctttaaga ctgatgaatg |
| 1741 | ctaccatcac tgaagaattg tttttcaaag gtctcattgg caatatacga attgacagtg |
| 1801 | ttatcccaca tattttgaaa atggagcctg cagattataa ctctcaaata attggtcaca |
| 1861 | gcatttgaaa actgtgactg cagtgctgta aacttaactg ttctttgcca gaacacaaga |
| 1921 | caccaaattg aactcactgc ttttgaggca tctggaaatt tttactttaa aaagtaacca |
| 1981 | gaatccaagg tatttttatt ttagcttccc ttaagaattt ttgaagtgac tgggcaggca |
| 2041 | gcagaaatta aatgaatttt tcttcctgat tcctttaaat gaatatgaaa cactacaaat |
| 2101 | ttattcttgg tgaagatgat acctgaagct gtcacctctt gattatctaa actaagcgct |
| 2161 | cattctattt tataaaacaa ataaattagt ctcttttttc tg |
| 10. SEQ ID NO:10 Genbank Accession No. M21985. Human steroid receptor | |
| TR2 protein. | |
| MATIEEIAHQIIEQQMGEIVTEQQTGQKIQIVTALDHNTQGKQF | |
| ILTNHDGSTPSKVILARQDSTPGKVFLTTPDAAGVNQLFFTTPDLSAQHLQLLTDNSP | |
| DQGPNKVFDLCVVCGDKASGRHYGAVTCEGCKGFFKRSIRKNLVYSCRGSKDCIINKH | |
| HRNRCQYCRLQRCIAFGMKQDSVQCERKPIEVSREKSSNCAASTEKIYIRKDLRSPLT | |
| ATPTFVTDSESTRSTGLLDSGMFMNIHPSGVKTESAVLMTSDKAESCQGDLSTLANVV | |
| TSLANLGKTKDLSQNSNEMSMIESLSNDDTSLCEFQEMQTNGDVSRAFDTLAKALNPG | |
| ESTACQSSVAGMEGSVHLITGDSSINYTEKEGPLLSDSHVAFRLTMPSPMPEYLNVHY | |
| IGESASRLLFLSMHWALSIPSFQALGQENSISLVKAYWNELFTLGLAQCWQVMNVATI | |
| LATFVNCLHNSLQQDAKVIAALIHFTRRAITDL″ | |
| 11. SEQ ID NO:11 Genbank Accession No. M21985. Human steroid receptor | |
| TR2 mRNA, complete cds encodes protein disclosed herein as M21985 | |
| 1 | gaattcgggc ccgtcggctt tcttcaaccc tctcttcccg gagcgccccc aatccacgag |
| 61 | tggcagccgc gggactgtcg cgtcggcgcc cgacgcggag tcagcagggg cgaaaagcgg |
| 121 | tagatcatgg caaccataga agaaattgca catcaaatta ttgaacaaca gatgggagag |
| 181 | attgttacag agcagcaaac tgggcagaaa atccagattg tgacagcact tgatcataat |
| 241 | acccaaggca agcagttcat tctgacaaat cacgacggct ctactccaag caaagtcatt |
| 301 | ctggccaggc aagattccac tccgggaaaa gttttcctta caactccaga tgcagcaggt |
| 361 | gtcaaccagt tattttttac cactcctgat ctgtctgcac aacacctgca gctcctaaca |
| 421 | gataattctc cagaccaagg accaaataag gtttttgatc tttgcgtagt atgtggagac |
| 481 | aaagcatcag gacgtcatta tggagcagta acttgtgaag gctgcaaagg attttttaaa |
| 541 | agaagcatcc gaaaaaattt agtatattca tgtcgaggat caaaggattg tattattaat |
| 601 | aagcaccacc gaaaccgctg tcaatactgc aggttacaga gatgtattgc gtttggaatg |
| 661 | aagcaagact ctgtccaatg tgaaagaaaa cccattgaag tatcacgaga aaaatcttcc |
| 721 | aactgtgccg cttcaacaga aaaaatctat atccgaaagg accttcgtag cccattaact |
| 781 | gcaactccaa cttttgtaac agatagtgaa agtacaaggt caacaggact gttagattca |
| 841 | ggaatgttca tgaatattca tccatctgga gtaaaaactg agtcagctgt gctgatgaca |
| 901 | tcagataagg ctgaatcatg tcagggagat ttaagtacat tggccaatgt ggttacatca |
| 961 | ttagcgaatc ttggaaaaac taaagatctt tctcaaaata gtaatgaaat gtctatgatt |
| 1021 | gaaagcttaa gcaatgatga tacctctttg tgtgaatttc aagaaatgca gaccaacggt |
| 1081 | gatgtttcaa gggcatttga cactcttgca aaagcattga atcctggaga gagcacagcc |
| 1141 | tgccagagct cagtagcggg catggaagga agtgtacacc taatcactgg agattcaagc |
| 1201 | ataaattaca ccgaaaaaga ggggccactt ctcagcgatt cacatgtagc tttcaggctc |
| 1261 | accatgcctt ctcctatgcc tgagtacctg aatgtgcact acattgggga gtctgcctcc |
| 1321 | agactgctgt tcttatcaat gcactgggca ctttcgattc cttctttcca ggctctaggg |
| 1381 | caagaaaaca gcatatcact ggtgaaagct tactggaatg aactttttac tcttggtctt |
| 1441 | gcccagtgct ggcaagtgat gaatgtagca actatattag caacatttgt caattgtctt |
| 1501 | cacaatagtc ttcaacaaga tgccaaggta attgcagccc tcattcattt cacaagacga |
| 1561 | gcaatcactg atttataaat gcttaactat agaatggctt atgactaccc aaaacagtgc |
| 1621 | cccatcaaca aatggggaaa attgcctttt gagctcagga ataatttata aattggggac |
| 1681 | taccttttag ttctttagca tattctattt cttattgttt tatataattt ttaaatcatt |
| 1741 | tgcttcctcc ttatgtttaa cagcagaggg gtaatcacct taaaatgtca tcaaaaatag |
| 1801 | atctactaga aggcagcatc acattcccat cttacttatg gactcctacc cctggttcat |
| 1861 | gtcttatatg cctgtaatgg ttataaagcc taccttcagg aaagctatgg ttgactaatt |
| 1921 | actaatggat gggttttaaa catgtccctc tacaataaat taaaatcttt caatgtttga |
| 1981 | atataatgtg gaggtgttta cctgagggcc tctctatctc cccgaattc |
| 12. SEQ ID NO:12 Genbank Accession No P49116. Human Orphan nuclear | |
| receptor TR4 (Orphan nuclear receptor TAK1) | |
| 1 | mtspspriqi istdsavasp qriqivtdqq tgqkiqivta vdasgspkqq filtspdgag |
| 61 | tgkvilaspe tssakqlift tsdnlvpgri qivtdsasve rllgktdvqr pqvveycvvc |
| 121 | gdkasgrhyg avscegckgf fkrsvrknlt yscrsnqdci inkhhrnrcq fcrlkkclem |
| 181 | gmkmesvqse rkpfdvqrek psncaastek iyirkdlrsp liatptfvad kdgarqtgll |
| 241 | dpgmlvniqq pliredgtvl latdskaets qgalgtlanv vtslanlses lnngdtseiq |
| 301 | pedqsaseit rafdtlakal nttdssssps ladgidtsgg gsihvisrdq stpiievegp |
| 361 | llsdthvtfk ltmpspmpey lnvhyicesa srllflsmhw arsipafqal gqdcntslvr |
| 421 | acwnelftlg laqcaqvmsl stilaaivnh lqnsiqedkl sgdrikqvme hiwklqefcn |
| 481 | smakididgy eyaylkaivl fspdhpglts tsqiekfqek aqmelqdyvq ktysedtyrl |
| 541 | arilvrlpal rlmssnitee lfftglignv sidsiipyil kmetaeyngq itgasl |
| 13. SEQ ID NO:13 Genbank Accession No. NP_059019. Rat TR4 orphan | |
| receptor; orphan receptor, TR4 [Rattus norvegicus] | |
| 1 | mtspspriqi istdsavrsp qriqivtdqq tgqklqivta vdasgsskqq filtspdgag |
| 61 | tgkvilaspe tssakqlift tsdnlvpgri qivtdsasve rllgkadvqr pqvveycvvc |
| 121 | gdkasgrhyg avscegckgf fkrsvrknlt yscrssqdci inkhhrnrcq fcrlkkclem |
| 181 | gmkmesvqse rkpfdvqrek psncaastek iyirkdlrsp liatptfvad kdgsrqtgll |
| 241 | dpgmlvniqq pliredgtvl latdskaets qgalgtlanv vtslanlses lnngdasemq |
| 301 | pedqsaseit rafdtlakal nttdsaspps ladgidasgg gsihvisrdq stpiievegp |
| 361 | llsdthvtfk ltmpspmpey lnvhyicesa srllflsmhw arsipafqal gqdcntslvr |
| 421 | acwnelftlg laqcaqvmsl stilaaivnh lqnsiqedkl sgdrikqvme hiwklqefcn |
| 481 | smakldidgh eyaylkaivl fspdhpgltg tsqiekfqek aqmelqdyvq ktysedtyrl |
| 541 | arilvrlpal rlmssnitee lfftglignv sidsiipyil kmetaeyngq itgasl |
| 14. SEQ ID NO:14 180177 TR4 orphan receptor-Rat | |
| 1 | mtspspriqi istdsavrsp qriqivtdqq tgqklqivta vdasgsskqq filtspdgag |
| 61 | tgkvilaspe tssakqlift tsdnlvpgri qivtdsasve rllgkadvqr pqvveycvvc |
| 121 | gdkasgrhyg avscegckgf fkrsvrknlt yscrssqdci inkhhrnrcq fcrlkkclem |
| 181 | gmkmesvqse rkpfdvqrek psncaastek iyirkdlrsp liatptfvad kdgsrqtgll |
| 241 | dpgmlvniqq pliredgtvl latdskaets qgalgtlanv vtslanlses lnngdasemq |
| 301 | pedqsaseit rafdtlakal nttdsaspps ladgidasgg gsihvisrdq stpiievegp |
| 361 | llsdthvtfk ltmpspmpey lnvhyicesa srllflsmhw arsipafqal gqdcntslvr |
| 421 | acwnelftlg Iaqcaqvmsl stilaaivnh lqnsiqedkl sgdrikqvme hiwklqefcn |
| 481 | smakldidgh eyaylkaivl fspdhpgltg tsqiekfqek aqmelqdyvq ktysedtyrl |
| 541 | arilvrlpal rlmssnitee lfftglignv sidsiipyil kmetaeyngq itgasl |
| 15. SEQ ID NO:15 159309 TR4 orphan receptor-human | |
| 1 | mtspspriqi istdsavasp qriqgsepas gplsvftsln kekivtdqqt gqkiqivtav |
| 61 | dasgspkqqf iltspdgagt gkvilaspet ssakqliftt sdnlvpgriq ivtdsasver |
| 121 | llgktdvqrp qvveycvvcg dkasgrhyga vscegckgff krsvrknlty scrssqdcii |
| 181 | nkhhrnrcqf crlkkclemg mkmesvqser kpfdvqrekp sncaasteki yirkdlrspl |
| 241 | iatptfvadk dgarqtglld pgmlvniqqp liredgtvll atdskaetsq galgtlanvv |
| 301 | tslanlsesl nngdtseiqp edqsaseitr afdtlakaln ttdsssspsl adgidtsggg |
| 361 | sihvisrdqs tpiievegpl lsdthvtfkl tmpspmpeyl nvhyicesas rllflsmhra |
| 421 | rsipafqglg qdcntslvra cwnelftlgl aqcaqvmsls tilaaivnhl qnsiqedkls |
| 481 | gdrikqvmeh iwklqefcns manwdidgye yaylkaivlf spdhpgltst sqiekfqeka |
| 541 | qmelqdyvqk tysedtyrla rilvrlpalr lmssniteel fftglignvs idsiipyilk |
| 601 | metaeyngqi tgvsl |
| 16. SEQ ID NO:16 TR4 nucleic acid Genbank Accession No: L27586. Human | |
| TR4 orphan receptor protein | |
| MTSPSPRIQIISTDSAVASPQRIQGSEPASGPLSVFTSLNKEKI | |
| VTDQQTGQKIQIVTAVDASGSPKQQFILTSPDGAGTGKVILASPETSSAKQLIFTTSD | |
| NLVPGRIQIVTDSASVERLLGKTDVQRPQVVEYCVVCGDKASGRHYGAVSCEGCKGFF | |
| KRSVRKNLTYSCRSSQDCIINKHHRNRCQFCRLKKCLEMGMKMESVQSERKPFDVQRE | |
| KPSNCAASTEKIYIRKDLRSPLIATPTFVADKDGARQTGLLDPGMLVNIQQPLIREDG | |
| TVLLATDSKAETSQGALGTLANVVTSLANLSESLNNGDTSEIQPEDQSASEITRAFDT | |
| LAKALNTTDSSSSPSLADGIDTSGGGSIHVISRDQSTPIIEVEGPLLSDTHVTFKLTM | |
| PSPMPEYLNVHYICESASRLLFLSMHRARSIPAFQGLGQDCNTSLVRACWNELFTLGL | |
| AQCAQVMSLSTILAAIVNHLQNSIQEDKLSGDRIKQVMEHIWKLQEFCNSMANWDIDG | |
| YEYAYLKAIVLFSPDHPGLTSTSQIEKFQEKAQMELQDYVQKTYSEDTYRLARILVRL | |
| PALRLMSSNITEELFFTGLIGNVSIDSIIPYILKMETAEYNGQITGVSL | |
| 17. SEQ ID NO:17 TR4 nucleic acid Genbank Accession No: L27586. Human | |
| TR4 orphan receptor mRNA, complete cds encoding TR4 protein L27586 | |
| 1 | gaattcgccg ctcccacctc ggcgtctcgt ctctcgcccg ctgccccgcg agcccgcggc |
| 61 | ccccggctcc cgccatccgc cgacaccggg agcccgggct ccccgcgccc tgccctccgc |
| 121 | gccgggggcc gcccgccgca gacacgggac ccgcttcgag gccgctttgg cgcaaatcct |
| 181 | gaggtaacac gtacacagac ctctcggccg gaatctccag ggatgaccag cccctcccca |
| 241 | cgcatccaga taatctccac cgactctgct gtagcctcac ctcagcgcat tcagggctct |
| 301 | gaacctgcct ctggcccatt gagtgttttc acatctttga acaaagagaa gattgtcaca |
| 361 | gaccagcaga caggacagaa aatccagata gtcaccgcag tggacgcctc cggatccccc |
| 421 | aaacagcagt tcatcctgac cagcccagat ggagctggaa ctgggaaggt gatcctggct |
| 481 | tccccagaga catccagcgc caagcaactc atattcacca cctcagacaa cctcgtccct |
| 541 | ggcaggatcc agattgtcac ggattctgcc tctgtggagc gtttactggg gaagacggac |
| 601 | gtccagcggc cccaggtggt agagtactgt gtggtctgtg gcgacaaagc ctccggccgt |
| 661 | cactatgggg ctgtcagttg tgaaggttgc aaaggtttct tcaaaaggag tgtgaggaaa |
| 721 | aatttgacct acagctgccg gagcagccaa gactgcatca tcaataaaca tcaccggaac |
| 781 | cgctgtcagt tttgccggct gaaaaaatgc ttagagatgg gcatgaaaat ggaatctgtg |
| 841 | cagagtgaac ggaagccctt cgatgtgcaa cgggagaaac caagcaattg tgctgcttca |
| 901 | actgagaaaa tctatatccg gaaagacctg agaagtcccc tgatagctac tcccacgttt |
| 961 | gtggcagaca aagatggagc aagacaaaca ggtcttcttg atccagggat gcttgtgaac |
| 1021 | atccagcagc ctttgatacg tgaggatggt acagttctcc tggccacgga ttctaaggct |
| 1081 | gaaacaagcc agggagctct gggcacactg gcaaatgtag tgacctccct tgccaaccta |
| 1141 | agtgaatctt tgaacaacgg tgacacttca gaaatccagc cagaggacca gtctgcaagt |
| 1201 | gagataactc gggcatttga taccttagct aaagcactta ataccacaga cagctcctct |
| 1261 | tctccaagct tggcagatgg gatagacacc agtggaggag ggagcatcca cgtcatcagc |
| 1321 | agagaccagt cgacacccat cattgaggtt gaaggccccc tcctttcaga cacacacgtc |
| 1381 | acatttaagc taacaatgcc cagtccaatg ccagagtacc tcaacgtgca ctacatctgt |
| 1441 | gagtctgcat cccgtctgct tttcctctca atgcaccggg caaggtcaat cccagccttt |
| 1501 | cagggacttg gacaggactg caacaccagc cttgtgcggg cctgctggaa tgagctcttc |
| 1561 | accctcggcc tggcccagtg tgcccaggtc atgagtctct ccaccatcct ggctgccatt |
| 1621 | gtcaaccacc tgcagaacag catccaggaa gataaacttt ctggtgaccg gataaagcaa |
| 1681 | gtcatggagc acatctggaa gctgcaggag ttctgtaaca gcatggcgaa ctgggatata |
| 1741 | gatggctatg agtatgcata ccttaaagct atagttctct ttagccccga tcatccaggt |
| 1801 | ttgaccagca caagccagat tgaaaaattc caagaaaagg cacagatgga gttgcaggac |
| 1861 | tatgttcaga aaacctactc agaagacacc taccgattgg cccggatcct cgttcgcctg |
| 1921 | ccggcactca ggctgatgag ctccaacata acagaagaac ttttttttac tggtctcatt |
| 1981 | ggcaatgttt cgatagacag cataatcccc tacatcctca agatggagac agcagagtat |
| 2041 | aatggccaga tcaccggagt cagtctatag cgcaaaccac acacctgcca aggagcaaca |
| 2101 | gaatccttcc aggaccgttc acatacaaag aaaagtagtg gtattttggt atgtgcaaat |
| 2161 | atttccatat gttagccatt tcctgtctgg tttctcctta tctgttaatc ccagacaata |
| 2221 | gcaattaaaa gactagtagg gatcctttcc tgac |
| 18. SEQ ID NO:18 TR4 nucleic acid Genbank Accession No: L27586. Human | |
| TR4 orphan receptor mRNA, degenerate sequence encoding TR4 protein | |
| L27586, position 228 C to T | |
| 1 | gaattcgccg ctcccacctc ggcgtctcgt ctctcgcccg ctgccccgcg agcccgcggc |
| 61 | ccccggctcc cgccatccgc cgacaccggg agcccgggct ccccgcgccc tgccctccgc |
| 121 | gccgggggcc gcccgccgca gacacgggac ccgcttcgag gccgctttgg cgcaaatcct |
| 181 | gaggtaacac gtacacagac ctctcggccg gaatctccag ggatgacTag cccctcccca |
| 241 | cgcatccaga taatctccac cgactctgct gtagcctcac ctcagcgcat tcagggctct |
| 301 | gaacctgcct ctggcccatt gagtgttttc acatctttga acaaagagaa gattgtcaca |
| 361 | gaccagcaga caggacagaa aatccagata gtcaccgcag tggacgcctc cggatccccc |
| 421 | aaacagcagt tcatcctgac cagcccagat ggagctggaa ctgggaaggt gatcctggct |
| 481 | tccccagaga catccagcgc caagcaactc atattcacca cctcagacaa cctcgtccct |
| 541 | ggcaggatcc agattgtcac ggattctgcc tctgtggagc gtttactggg gaagacggac |
| 601 | gtccagcggc cccaggtggt agagtactgt gtggtctgtg gcgacaaagc ctccggccgt |
| 661 | cactatgggg ctgtcagttg tgaaggttgc aaaggtttct tcaaaaggag tgtgaggaaa |
| 721 | aatttgacct acagctgccg gagcagccaa gactgcatca tcaataaaca tcaccggaac |
| 781 | cgctgtcagt tttgccggct gaaaaaatgc ttagagatgg gcatgaaaat ggaatctgtg |
| 841 | cagagtgaac ggaagccctt cgatgtgcaa cgggagaaac caagcaattg tgctgcttca |
| 901 | actgagaaaa tctatatccg gaaagacctg agaagtcccc tgatagctac tcccacgttt |
| 961 | gtggcagaca aagatggagc aagacaaaca ggtcttcttg atccagggat gcttgtgaac |
| 1021 | atccagcagc ctttgatacg tgaggatggt acagttctcc tggccacgga ttctaaggct |
| 1081 | gaaacaagcc agggagctct gggcacactg gcaaatgtag tgacctccct tgccaaccta |
| 1141 | agtgaatctt tgaacaacgg tgacacttca gaaatccagc cagaggacca gtctgcaagt |
| 1201 | gagataactc gggcatttga taccttagct aaagcactta ataccacaga cagctcctct |
| 1261 | tctccaagct tggcagatgg gatagacacc agtggaggag ggagcatcca cgtcatcagc |
| 1321 | agagaccagt cgacacccat cattgaggtt gaaggccccc tcctttcaga cacacacgtc |
| 1381 | acatttaagc taacaatgcc cagtccaatg ccagagtacc tcaacgtgca ctacatctgt |
| 1441 | gagtctgcat cccgtctgct tttcctctca atgcaccggg caaggtcaat cccagccttt |
| 1501 | cagggacttg gacaggactg caacaccagc cttgtgcggg cctgctggaa tgagctcttc |
| 1561 | accctcggcc tggcccagtg tgcccaggtc atgagtctct ccaccatcct ggctgccatt |
| 1621 | gtcaaccacc tgcagaacag catccaggaa gataaacttt ctggtgaccg gataaagcaa |
| 1681 | gtcatggagc acatctggaa gctgcaggag ttctgtaaca gcatggcgaa ctgggatata |
| 1741 | gatggctatg agtatgcata ccttaaagct atagttctct ttagccccga tcatccaggt |
| 1801 | ttgaccagca caagccagat tgaaaaattc caagaaaagg cacagatgga gttgcaggac |
| 1861 | tatgttcaga aaacctactc agaagacacc taccgattgg cccggatcct cgttcgcctg |
| 1921 | ccggcactca ggctgatgag ctccaacata acagaagaac ttttttttac tggtctcatt |
| 1981 | ggcaatgttt cgatagacag cataatcccc tacatcctca agatggagac agcagagtat |
| 2041 | aatggccaga tcaccggagt cagtctatag cgcaaaccac acacctgcca aggagcaaca |
| 2101 | gaatccttcc aggaccgttc acatacaaag aaaagtagtg gtattttggt atgtgcaaat |
| 2161 | atttccatat gttagccatt tcctgtctgg tttctcctta tctgttaatc ccagacaata |
| 2221 | gcaattaaaa gactagtagg gatcctttcc tgac |
| 19. SEQ ID NO:19 TR4 nucleic acid Genbank Accession No: L27586. Human | |
| TR4 orphan receptor protein Conserved variant I position 8 to a V | |
| MTSPSPRvQIISTDSAVASPQRIQGSEPASGPLSVFTSLNKEKI | |
| VTDQQTGQKIQIVTAVDASGSPKQQFILTSPDGAGTGKVILASPETSSAKQLIFTTSD | |
| NLVPGRIQIVTDSASVERLLGKTDVQRPQVVEYCVVCGDKASGRHYGAVSCEGCKGFF | |
| KRSVRKNLTYSCRSSQDCIINKHHRNRCQFCRLKKCLEMGMKMESVQSERKPFDVQRE | |
| KPSNCAASTEKIYIRKDLRSPLIATPTFVADKDGARQTGLLDPGMLVNIQQPLIREDG | |
| TVLLATDSKAETSQGALGTLANVVTSLANLSESLNNGDTSEIQPEDQSASEITRAFDT | |
| LAKALNTTDSSSSPSLADGIDTSGGGSIHVISRDQSTPIIEVEGPLLSDTHVTFKLTM | |
| PSPMPEYLNVHYICESASRLLFLSMHRARSIPAFQGLGQDCNTSLVRACWNELFTLGL | |
| AQCAQVMSLSTILAAIVNHLQNSIQEDKLSGDRIKQVMEHIWKLQEFCNSMANWDIDG | |
| YEYAYLKAIVLFSPDHPGLTSTSQIEKFQEKAQMELQDYVQKTYSEDTYRLARILVRL | |
| PALRLMSSNITEELFFTGLIGNVSIDSIIPYILKMETAEYNGQITGVSL | |
| 20. SEQ ID NO:20 TR4 nucleic acid encoding SEQ ID NO:19. I to V mutant | |
| has c to T at 228 and A to G change at position 244. | |
| 1 | gaattcgccg ctcccacctc ggcgtctcgt ctctcgcccg ctgccccgcg agcccgcggc |
| 61 | ccccggctcc cgccatccgc cgacaccggg agcccgggct ccccgcgccc tgccctccgc |
| 121 | gccgggggcc gcccgccgca gacacgggac ccgcttcgag gccgctttgg cgcaaatcct |
| 181 | gaggtaacac gtacacagac ctctcggccg gaatctccag ggatgacTag cccctcccca |
| 241 | cgcGtccaga taatctccac cgactctgct gtagcctcac ctcagcgcat tcagggctct |
| 301 | gaacctgcct ctggcccatt gagtgttttc acatctttga acaaagagaa gattgtcaca |
| 361 | gaccagcaga caggacagaa aatccagata gtcaccgcag tggacgcctc cggatccccc |
| 421 | aaacagcagt tcatcctgac cagcccagat ggagctggaa ctgggaaggt gatcctggct |
| 481 | tccccagaga catccagcgc caagcaactc atattcacca cctcagacaa cctcgtccct |
| 541 | ggcaggatcc agattgtcac ggattctgcc tctgtggagc gtttactggg gaagacggac |
| 601 | gtccagcggc cccaggtggt agagtactgt gtggtctgtg gcgacaaagc ctccggccgt |
| 661 | cactatgggg ctgtcagttg tgaaggttgc aaaggtttct tcaaaaggag tgtgaggaaa |
| 721 | aatttgacct acagctgccg gagcagccaa gactgcatca tcaataaaca tcaccggaac |
| 781 | cgctgtcagt tttgccggct gaaaaaatgc ttagagatgg gcatgaaaat ggaatctgtg |
| 841 | cagagtgaac ggaagccctt cgatgtgcaa cgggagaaac caagcaattg tgctgcttca |
| 901 | actgagaaaa tctatatccg gaaagacctg agaagtcccc tgatagctac tcccacgttt |
| 961 | gtggcagaca aagatggagc aagacaaaca ggtcttcttg atccagggat gcttgtgaac |
| 1021 | atccagcagc ctttgatacg tgaggatggt acagttctcc tggccacgga ttctaaggct |
| 1081 | gaaacaagcc agggagctct gggcacactg gcaaatgtag tgacctccct tgccaaccta |
| 1141 | agtgaatctt tgaacaacgg tgacacttca gaaatccagc cagaggacca gtctgcaagt |
| 1201 | gagataactc gggcatttga taccttagct aaagcactta ataccacaga cagctcctct |
| 1261 | tctccaagct tggcagatgg gatagacacc agtggaggag ggagcatcca cgtcatcagc |
| 1321 | agagaccagt cgacacccat cattgaggtt gaaggccccc tcctttcaga cacacacgtc |
| 1381 | acatttaagc taacaatgcc cagtccaatg ccagagtacc tcaacgtgca ctacatctgt |
| 1441 | gagtctgcat cccgtctgct tttcctctca atgcaccggg caaggtcaat cccagccttt |
| 1501 | cagggacttg gacaggactg caacaccagc cttgtgcggg cctgctggaa tgagctcttc |
| 1561 | accctcggcc tggcccagtg tgcccaggtc atgagtctct ccaccatcct ggctgccatt |
| 1621 | gtcaaccacc tgcagaacag catccaggaa gataaacttt ctggtgaccg gataaagcaa |
| 1681 | gtcatggagc acatctggaa gctgcaggag ttctgtaaca gcatggcgaa ctgggatata |
| 1741 | gatggctatg agtatgcata ccttaaagct atagttctct ttagccccga tcatccaggt |
| 1801 | ttgaccagca caagccagat tgaaaaattc caagaaaagg cacagatgga gttgcaggac |
| 1861 | tatgttcaga aaacctactc agaagacacc taccgattgg cccggatcct cgttcgcctg |
| 1921 | ccggcactca ggctgatgag ctccaacata acagaagaac ttttttttac tggtctcatt |
| 1981 | ggcaatgttt cgatagacag cataatcccc tacatcctca agatggagac agcagagtat |
| 2041 | aatggccaga tcaccggagt cagtctatag cgcaaaccac acacctgcca aggagcaaca |
| 2101 | gaatccttcc aggaccgttc acatacaaag aaaagtagtg gtattttggt atgtgcaaat |
| 2161 | atttccatat gttagccatt tcctgtctgg tttctcctta tctgttaatc ccagacaata |
| 2221 | gcaattaaaa gactagtagg gatcctttcc tgac |
| 21. SEQ ID NO:21 Genbank Accession No. X80172. M.musculus gene for | |
| androgen-receptor 5′ untranslated region. | |
| 1 | ctgcagcttg ttctttaatg tcaggagact ctcccttctg cttgtcctgg tgggccctgg |
| 61 | ggggagcggg gagggaatac ctaagagcaa ttggtagctg gtacttctaa tgcctcttcc |
| 121 | tcctccaacc tccaagagtc tgttttggga ttgggttcag gaatgaaatt ctgcctgtgc |
| 181 | taacctcctg gggagccggt agacttgtct gttaaaaatc gcttctgctt ttggagccta |
| 241 | aagcccggtt ccgaaaaaca agtggtattt aggggaaaga ggggtcttca aaggctacag |
| 301 | tgagtcattc cagccttcaa ccatactacg ccagcactac gttctctaaa gccactctgc |
| 361 | gctagcttgc ggtgagggga ggggagaaaa ggaaagggga ggggagggga ggggagggag |
| 421 | aaaggaggtg ggaaggcaga gaggccggct gcgggggcgg gaccgactca caaactgttc |
| 481 | gatttcgttt ccacctccca gcgccccctc ggagatccct aggagccagc ctgctgggag |
| 541 | aaccagaggg tccggagcaa acctggaggc tgagagggca tcagagggga aaagactgag |
| 601 | ctagccactc cagtgccata cagaagctta agggacgcac cacgccagcc ccagcccagc |
| 661 | gacagccaac gcctgttgca gagcggcggc ttcgaagccg ccgcccagga gctgcccttt |
| 721 | cctcttcggt gaagtttcta aaagctgcgg gagactcaga ggaagcaagg aaagtgtccg |
| 781 | gtaggactac ggctgccttt gtcctcttcc cctctaccct taccccctcc tgggtcccct |
| 841 | ctccaggagc tgactaggca ggctttctgg ccaaccctct cccctacacc cccagctctg |
| 901 | ccagccagtt tgcacagagg taaactccct ttggctgaga gtaggggagc ttgttgcaca |
| 961 | ttgcaaggaa ggcttttggg agcccagaga ctgaggagca acagcacgcc caggagagtc |
| 1021 | cctggttcca ggttctcgcc cctgcacctc ctcctgcccg cccctcaccc tgtgtgtggt |
| 1081 | gttagaaatg aaaagatgaa aaggcagcta gggtttcagt agtcgaaagc aaaacaaaag |
| 1141 | ctaaaagaaa acaaaaagaa aatagcccag ttcttatttg cacctgcttc agtggacttt |
| 1201 | gaatttggaa ggcagaggat ttcccctttt ccctcccgtc aaggtttgag catcttttaa |
| 1261 | tctgttcttc aagtatttag agacaaactg tgtaagtagc agggcagatc ctgtcttgcg |
| 1321 | cgtgccttcc tttactggag actttgaggt tatctgggca ctccccccac ccaccccccc |
| 1381 | tcctgcaagt tttcttcccc ggagcttccc gcaggtgggc agctagctgc agatactaca |
| 1441 | tcatcagtca ggagaactct tcagagcaag agacgaggag gcaggataag ggaattc |
| 22. SEQ ID NO:22 Genbank Accession No. X59591. Mouse gene for androgen | |
| receptor promoter region. | |
| 1 | ctgcagcttg ttctttaatg tcaggagact ctcccttctg cttgtcctgg tgggccctgg |
| 61 | ggggagcggg gagggaatac ctaagagcaa ttggtagctg gtacttctaa tgcctcttcc |
| 121 | tcctccaacc tccaagagtc tgttttggga ttgggttcag gaatgaaatt ctgcctgtgc |
| 181 | taacctcctg gggagccggt agacttgtct gttaaaaatc gcttctgctt ttggagccta |
| 241 | aagcccggtt ccgaaaaaca agtggtattt aggggaaaga ggggtcttca aaggctacag |
| 301 | tgagtcattc cagccttcaa ccatactacg ccagcactac gttctctaaa gccactctgc |
| 361 | gctagcttgc ggtgagggga ggggagaaaa ggaaagggga ggggagggga ggggagggag |
| 421 | aaaggaggtg ggaaggcaga gaggccggct gcgggggcgg gaccgactca caaactgttc |
| 481 | gatttcgttt ccacctccca gcgccccctc ggagatccct aggagccagc ctgctgggag |
| 541 | aaccagaggg tccggagcaa acctggaggc tgagagggca tcagagggga aaagactgag |
| 23. SEQ ID NO:23 Genbank Accession No. X59590. Mouse gene for androgen | |
| receptor, 3′ UTR. | |
| 1 | cccaagcgct agtgttctgt tctctttttg taatcttgga atcttttgtt gctctaaata |
| 61 | caattaaaaa tggcagaaac ttgtttgttg gaatacatgt gtgactcttg gtttgtctct |
| 121 | gcgtctggct ttagaaatgt catccattgt gtaaaatact ggcttgttgg tctgccagct |
| 181 | aaaacttgcc acagcccctg ttgtgactgc aggctcaagt tattgttaac aaagagcccc |
| 241 | aagaaaagct gctaatgtcc tcttatcacc attgttaatt tgttaaaaca taaaacaatc |
| 301 | taaaatttca gatgaatgtc atcagagttc ttttcattag ctctttttat tggctgtct |
| 24. SEQ ID NO:24 Genbank Accession No. X59592. Mouse protein for | |
| androgen receptor. | |
| MEVQLGLGRVYPRPPSKTYRGAFQNLFQSVREAIQNPGPRHPEA | |
| ANIAPPGACLQQRQETSPRRRRRQQHTEDGSPQAHIRGPTGYLALEEEQQPSQQQAAS | |
| EGHPESSCLPEPGAATAPGKGLPQQPPAPPDQDDSAAPSTLSLLGPTFPGLSSCSADI | |
| KDILNEAGTMQLLQQQQQQQQHQQQHQQHQQQQEVISEGSSARAREATGAPSSSKDSY | |
| LGGNSTISDSAKELCKAVSVSMGLGVEALEHLSPGEQLRGDCMYASLLGGPPAVRPTP | |
| CAPLPECKGLPLDEGPGKSTEETAEYSSFKGGYAKGLEGESLGCSGSSEAGSSGTLEI | |
| PSSLSLYKSGALDEAAAYQNRDYYNFPLALSGPPHPPPPTHPHARIKLENPLDYGSAW | |
| AAAAAQCRYGDLGSLHGGSVAGPSTGSPPATTSSSWHTLFTAEEGQLYGPGGGGGSSS | |
| PSDAGPVAPYGYTRPPQGLTSQESDYSASEVWYPGGVVNRVPYPSPNCVKSEMGPWME | |
| NYSGPYGDMRLDSTRDHVLPIDYYFPPQKTCLICGDEASGCHYGALTCGSCKVFFKRA | |
| AEGKQKYLCASRNDCTIDKFRRKNCPSCRLRKCYEAGMTLGARKLKKLGNLKLQEEGE | |
| NSNAGSPTEDPSQKMTVSHIEGYECQPIFLNVLEAIEPGVVCAGHDNNQPDSFAALLS | |
| SLNELGERQLVHVVKWAKALPGFRNLHVDDQMAVIQYSWMGLMVFAMGWRSFTNVNSR | |
| MLYFAPDLVFNEYRMHKSRMYSQCVRMRHLSQEFGWLQITPQEFLCMKALLLFSIIPV | |
| DGLKNQKFFDELRMNYIKELDRIIACKRKNPTSCSRRFYQLTKLLDSVQPIARELHQF | |
| TFDLLIKSHMVSVDFPEMMAEIISVQVPKILSGKVKPIYFHTQ″ | |
| 25. SEQ ID NO:25 Genbank Accession No. X59592. Mouse mRNA for androgen | |
| receptor | |
| 1 | gcttcccgca ggtgggcagc tagctgcaga tactacatca tcagtcagga gaactcttca |
| 61 | gagcaagaga cgaggaggca ggataaggga attcggtgga agctacagac aagctcaagg |
| 121 | atggaggtgc agttagggct gggaagggtc tacccacggc ccccatccaa gacctatcga |
| 181 | ggagcgttcc agaatctgtt ccagagcgtg cgcgaagcga tccagaaccc gggccccagg |
| 241 | caccctgagg ccgctaacat agcacctccc ggcgcctgtt tacagcagag gcaggagact |
| 301 | agcccccggc ggcggcggcg gcagcagcac actgaggatg gttctcctca agcccacatc |
| 361 | agaggcccca caggctacct ggccctggag gaggaacagc agccttcaca gcagcaggca |
| 421 | gcctccgagg gccaccctga gagcagctgc ctccccgagc ctggggcggc caccgctcct |
| 481 | ggcaaggggc tgccgcagca gccaccagct cctccagatc aggatgactc agctgcccca |
| 541 | tccacgttgt ccctgctggg ccccactttc ccaggcttaa gcagctgctc cgccgacatt |
| 601 | aaagacattt tgaacgaggc cggcaccatg caacttcttc agcagcagca acaacagcag |
| 661 | cagcaccaac agcagcacca acagcaccaa cagcagcagg aggtaatctc cgaaggcagc |
| 721 | agcgcaagag ccagggaggc cacgggggct ccctcttcct ccaaggatag ttacctaggg |
| 781 | ggcaattcaa ccatatctga cagtgccaag gagttgtgta aagcagtgtc tgtgtccatg |
| 841 | ggattgggtg tggaagcatt ggaacatctg agtccagggg aacagcttcg gggagactgc |
| 901 | atgtacgcgt cgctcctggg aggtccaccc gcggtgcgtc ccactccttg tgcgccgctg |
| 961 | cccgaatgca aaggtcttcc cctggacgaa ggcccaggca aaagcactga agagactgct |
| 1021 | gagtattcct ctttcaaggg aggttacgcc aaaggattgg aaggtgagag cttggggtgc |
| 1081 | tctggcagca gtgaagcagg tagctctggg acacttgaga tcccgtcctc tctgtctctg |
| 1141 | tataaatctg gagcactaga cgaggcagca gcataccaga atcgcgacta ctacaacttt |
| 1201 | ccgctggctc tgtccgggcc gccgcacccc ccgcccccta cccatccaca cgcccgtatc |
| 1261 | aagctggaga acccattgga ctacggcagc gcctgggctg cggcggcagc gcaatgccgc |
| 1321 | tatggggact tgggtagtct acatggaggg agtgtagccg ggcccagcac tggatcgccc |
| 1381 | ccagccacca cctcttcttc ctggcatact ctcttcacag ctgaagaagg ccaattatat |
| 1441 | gggccaggag gcgggggcgg cagcagcagc ccaagcgatg ccgggcctgt agccccctat |
| 1501 | ggctacactc ggccccctca ggggctgaca agccaggaga gtgactactc tgcctccgaa |
| 1561 | gtgtggtatc ctggtggagt tgtgaacaga gtaccctatc ccagtcccaa ttgtgtcaaa |
| 1621 | agtgaaatgg gaccttggat ggagaactac tccggacctt atggggacat gcgtttggac |
| 1681 | agtaccaggg accatgtttt acccatcgac tattactttc caccccagaa gacctgcctg |
| 1741 | atctgtggag atgaagcttc tggctgtcac tacggagctc tcacttgtgg cagctgcaag |
| 1801 | gtcttcttca aaagagccgc tgaagggaaa cagaagtatc tatgtgccag cagaaacgat |
| 1861 | tgtaccattg ataaatttcg gaggaaaaat tgcccatctt gtcgtctccg gaaatgttat |
| 1921 | gaagcaggga tgactctggg agctcgtaag ctgaagaaac ttggaaatct aaaactacag |
| 1981 | gaggaaggag aaaactccaa tgctggcagc cccactgagg acccatccca gaagatgact |
| 2041 | gtatcacaca ttgaaggcta tgaatgtcag cctatctttc ttaacgtcct ggaagccatt |
| 2101 | gagccaggag tggtgtgtgc cggacatgac aacaaccaac cagattcctt tgctgccttg |
| 2161 | ttatctagcc tcaatgagct tggagagagg cagcttgtgc atgtggtcaa gtgggccaag |
| 2221 | gccttgcctg gcttccgcaa cttgcatgtg gatgaccaga tggcggtcat tcagtattcc |
| 2281 | tggatgggac tgatggtatt tgccatgggt tggcggtcct tcactaatgt caactccagg |
| 2341 | atgctctact ttgcacctga cttggttttc aatgagtacc gcatgcacaa gtctcggatg |
| 2401 | tacagccagt gtgtgaggat gaggcacctg tctcaagagt ttggatggct ccaaataacc |
| 2461 | ccccaggaat tcctgtgcat gaaagcactg ctgctcttca gcattattcc agtggatggg |
| 2521 | ctgaaaaatc aaaaattctt tgatgaactt cgaatgaact acatcaagga actcgatcgc |
| 2581 | atcattgcat gcaaaagaaa gaatcccaca tcctgctcaa ggcgcttcta ccagctcacc |
| 2641 | aagctcctgg attctgtgca gcctattgca agagagctgc atcagttcac ttttgacctg |
| 2701 | ctaatcaagt cccatatggt gagcgtggac tttcctgaaa tgatggcaga gatcatctct |
| 2761 | gtgcaagtgc ccaagatcct ttctgggaaa gtcaagccca tctatttcca cacacagtga |
| 2821 | agatttggaa accctaatac ccaaaaccca ccttgttccc tttccagatg tcttctgcct |
| 2881 | gttatataac tctgcactac ttctctgcag tgccttgggg gaaattcctc tactgatgta |
| 2941 | cagtcagacg tgaacaggtt cctcagttct atttcctggg cttctcct |
| 26. SEQ ID NO:26 Genbank Accession No. X59592. Mouse protein for | |
| androgen receptor | |
| MEVQLGLGRVYPRPPSKTYRGAFQNLFQSVREAIQNPGPRHPEA | |
| ANIAPPGACLQQRQETSPRRRRRQQHTEDGSPQAHIRGPTGYLALEEEQQPSQQQAAS | |
| EGHPESSCLPEPGAATAPGKGLPQQPPAPPDQDDSAAPSTLSLLGPTFPGLSSCSADI | |
| KDILNEAGTMQLLQQQQQQQQHQQQHQQHQQQQEVISEGSSARAREATGAPSSSKDSY | |
| LGGNSTISDSAKELCKAVSVSMGLGVEALEHLSPGEQLRGDCMYASLLGGPPAVRPTP | |
| CAPLPECKGLPLDEGPGKSTEETAEYSSFKGGYAKGLEGESLGCSGSSEAGSSGTLEI | |
| PSSLSLYKSGALDEAAAYQNRDYYNFPLALSGPPHPPPPTHPHARIKLENPLDYGSAW | |
| AAAAAQCRYGDLGSLHGGSVAGPSTGSPPATTSSSWHTLFTAEEGQLYGPGGGGGSSS | |
| PSDAGPVAPYGYTRPPQGLTSQESDYSASEVWYPGGVVNRVPYPSPNCVKSEMGPWME | |
| NYSGPYGDMRLDSTRDHVLPIDYYFPPQKTCLICGDEASGCHYGALTCGSCKVFFKRA | |
| AEGKQKYLCASRNDCTIDKFRRKNCPSCRLRKCYEAGMTLGARKLKKLGNLKLQEEGE | |
| NSNAGSPTEDPSQKMTVSHIEGYECQPIFLNVLEAIEPGVVCAGHDNNQPDSFAALLS | |
| SLNELGERQLVHVVKWAKALPGFRNLHVDDQMAVIQYSWMGLMVFAMGWRSFTNVNSR | |
| MLYFAPDLVFNEYRMHKSRMYSQCVRMRHLSQEFGWLQITPQEFLCMKALLLFSIIPV | |
| DGLKNQKFFDELRMNYIKELDRIIACKRKNPTSCSRRFYQLTKLLDSVQPIARELHQF | |
| TFDLLIKSHMVSVDFPEMMAEIISVQVPKILSGKVKPIYFHTQ″ | |
| 27. SEQ ID NO:27 Genbank Accession No. X59592. Mouse mRNA for androgen | |
| receptor. | |
| 1 | gcttcccgca ggtgggcagc tagctgcaga tactacatca tcagtcagga gaactcttca |
| 61 | gagcaagaga cgaggaggca ggataaggga attcggtgga agctacagac aagctcaagg |
| 121 | atggaggtgc agttagggct gggaagggtc tacccacggc ccccatccaa gacctatcga |
| 181 | ggagcgttcc agaatctgtt ccagagcgtg cgcgaagcga tccagaaccc gggccccagg |
| 241 | caccctgagg ccgctaacat agcacctccc ggcgcctgtt tacagcagag gcaggagact |
| 301 | agcccccggc ggcggcggcg gcagcagcac actgaggatg gttctcctca agcccacatc |
| 361 | agaggcccca caggctacct ggccctggag gaggaacagc agccttcaca gcagcaggca |
| 421 | gcctccgagg gccaccctga gagcagctgc ctccccgagc ctggggcggc caccgctcct |
| 481 | ggcaaggggc tgccgcagca gccaccagct cctccagatc aggatgactc agctgcccca |
| 541 | tccacgttgt ccctgctggg ccccactttc ccaggcttaa gcagctgctc cgccgacatt |
| 601 | aaagacattt tgaacgaggc cggcaccatg caacttcttc agcagcagca acaacagcag |
| 661 | cagcaccaac agcagcacca acagcaccaa cagcagcagg aggtaatctc cgaaggcagc |
| 721 | agcgcaagag ccagggaggc cacgggggct ccctcttcct ccaaggatag ttacctaggg |
| 781 | ggcaattcaa ccatatctga cagtgccaag gagttgtgta aagcagtgtc tgtgtccatg |
| 841 | ggattgggtg tggaagcatt ggaacatctg agtccagggg aacagcttcg gggagactgc |
| 901 | atgtacgcgt cgctcctggg aggtccaccc gcggtgcgtc ccactccttg tgcgccgctg |
| 961 | cccgaatgca aaggtcttcc cctggacgaa ggcccaggca aaagcactga agagactgct |
| 1021 | gagtattcct ctttcaaggg aggttacgcc aaaggattgg aaggtgagag cttggggtgc |
| 1081 | tctggcagca gtgaagcagg tagctctggg acacttgaga tcccgtcctc tctgtctctg |
| 1141 | tataaatctg gagcactaga cgaggcagca gcataccaga atcgcgacta ctacaacttt |
| 1201 | ccgctggctc tgtccgggcc gccgcacccc ccgcccccta cccatccaca cgcccgtatc |
| 1261 | aagctggaga acccattgga ctacggcagc gcctgggctg cggcggcagc gcaatgccgc |
| 1321 | tatggggact tgggtagtct acatggaggg agtgtagccg ggcccagcac tggatcgccc |
| 1381 | ccagccacca cctcttcttc ctggcatact ctcttcacag ctgaagaagg ccaattatat |
| 1441 | gggccaggag gcgggggcgg cagcagcagc ccaagcgatg ccgggcctgt agccccctat |
| 1501 | ggctacactc ggccccctca ggggctgaca agccaggaga gtgactactc tgcctccgaa |
| 1561 | gtgtggtatc ctggtggagt tgtgaacaga gtaccctatc ccagtcccaa ttgtgtcaaa |
| 1621 | agtgaaatgg gaccttggat ggagaactac tccggacctt atggggacat gcgtttggac |
| 1681 | agtaccaggg accatgtttt acccatcgac tattactttc caccccagaa gacctgcctg |
| 1741 | atctgtggag atgaagcttc tggctgtcac tacggagctc tcacttgtgg cagctgcaag |
| 1801 | gtcttcttca aaagagccgc tgaagggaaa cagaagtatc tatgtgccag cagaaacgat |
| 1861 | tgtaccattg ataaatttcg gaggaaaaat tgcccatctt gtcgtctccg gaaatgttat |
| 1921 | gaagcaggga tgactctggg agctcgtaag ctgaagaaac ttggaaatct aaaactacag |
| 1981 | gaggaaggag aaaactccaa tgctggcagc cccactgagg acccatccca gaagatgact |
| 2041 | gtatcacaca ttgaaggcta tgaatgtcag cctatctttc ttaacgtcct ggaagccatt |
| 2101 | gagccaggag tggtgtgtgc cggacatgac aacaaccaac cagattcctt tgctgccttg |
| 2161 | ttatctagcc tcaatgagct tggagagagg cagcttgtgc atgtggtcaa gtgggccaag |
| 2221 | gccttgcctg gcttccgcaa cttgcatgtg gatgaccaga tggcggtcat tcagtattcc |
| 2281 | tggatgggac tgatggtatt tgccatgggt tggcggtcct tcactaatgt caactccagg |
| 2341 | atgctctact ttgcacctga cttggttttc aatgagtacc gcatgcacaa gtctcggatg |
| 2401 | tacagccagt gtgtgaggat gaggcacctg tctcaagagt ttggatggct ccaaataacc |
| 2461 | ccccaggaat tcctgtgcat gaaagcactg ctgctcttca gcattattcc agtggatggg |
| 2521 | ctgaaaaatc aaaaattctt tgatgaactt cgaatgaact acatcaagga actcgatcgc |
| 2581 | atcattgcat gcaaaagaaa gaatcccaca tcctgctcaa ggcgcttcta ccagctcacc |
| 2641 | aagctcctgg attctgtgca gcctattgca agagagctgc atcagttcac ttttgacctg |
| 2701 | ctaatcaagt cccatatggt gagcgtggac tttcctgaaa tgatggcaga gatcatctct |
| 2761 | gtgcaagtgc ccaagatcct ttctgggaaa gtcaagccca tctatttcca cacacagtga |
| 2821 | agatttggaa accctaatac ccaaaaccca ccttgttccc tttccagatg tcttctgcct |
| 2881 | gttatataac tctgcactac ttctctgcag tgccttgggg gaaattcctc tactgatgta |
| 2941 | cagtcagacg tgaacaggtt cctcagttct atttcctggg cttctcct |
| 28. SEQ ID NO:28 Genbank Accession No. M37890. Mouse androgen receptor | |
| protein, complete cds. | |
| MEVQLGLGRVYPRPPSKTYRGAFQNLFQSVREAIQNPGPRHPEA | |
| ANIAPPGACLQQRQETSPRRRRRQQHTEDGSPQAHIRGPTGYLALEEEQQPSQQQAAS | |
| EGHPESSCLPEPGAATAPGKGLPQQPPAPPDQDDSAAPSTLSLLGPTFPGLSSCSADI | |
| KDILNEAGTMQLLQQQQQQQQHQQQHQQHQQQQEVISEGSSARAREATGAPSSSKDSY | |
| LGGNSTISDSAKELCKAVSVSMGLGVEALEHLSPGEQLRGDCMYASLLGGPPAVRPTP | |
| CAPLPECKGLPLDEGPGKSTEETAEYSSFKGGYAKGLEGESLGCSGSSEAGSSGTLEI | |
| PSSLSLYKSGALDEAAAYQNRDYYNFPLALSGPPHPPPPTHPHARIKLENPLDYGSAW | |
| AAAAAQCRYGDLGSLHGGSVAGPSTGSPPATTSSSWHTLFTAEEGQLYGPGGGGGSSS | |
| PSDAGPVAPYGYTRPPQGLTSQESDYSASEVWYPGGVVNRVPYPSPNCVKSEMGPWME | |
| NYSGPYGDMRLDSTRDHVLPIDYYFPPQKTCLICGDEASGCHYGALTCGSCKVFFKRA | |
| AEGKQKYLCASRNDCTIDKFRRKNCPSCRLRKCYEAGMTLGARKLKKLGNLKLQEEGE | |
| NSNAGSPTEDPSQKMTVSHIEGYECQPIFLNVLEAIEPGVVCAGHDNNQPDSFAALLS | |
| SLNELGERQLVHVVKWAKALPGFRNLHVDDQMAVIQYSWMGLMVFAMGWRSFTNVNSR | |
| MLYFAPDLVFNEYRMHKSRMYSQCVRMRHLSQEFGWLQITPQEFLCMKALLLFSIIPV | |
| DGLKNQKFFDELRMNYIKELDRIIACKRKNPTSCSRRFYQLTKLLDSVQPIARELHQF | |
| TFDLLIKSHMVSVDFPEMMAEIISVQVPKILSGKVKPIYFHTQ | |
| 29. SEQ ID NO:29 Genbank Accession No. M37890. Mouse androgen receptor | |
| mRNA, complete cds | |
| 1 | atggaggtgc agttagggct gggaagggtc tacccacggc ccccatccaa gacctatcga |
| 61 | ggagcgttcc agaatctgtt ccagagcgtg cgcgaagcga tccagaaccc gggccccagg |
| 121 | caccctgagg ccgctaacat agcacctccc ggcgcctgtt tacagcagag gcaggagact |
| 181 | agcccccggc ggcggcggcg gcagcagcac actgaggatg gttctcctca agcccacatc |
| 241 | agaggcccca caggctacct ggccctggag gaggaacagc agccttcaca gcagcaggca |
| 301 | gcctccgagg gccaccctga gagcagctgc ctccccgagc ctggggcggc caccgctcct |
| 361 | ggcaaggggc tgccgcagca gccaccagct cctccagatc aggatgactc agctgcccca |
| 421 | tccacgttgt ccctgctggg ccccactttc ccaggcttaa gcagctgctc cgccgacatt |
| 481 | aaagacattt tgaacgaggc cggcaccatg caacttcttc agcagcagca acaacagcag |
| 541 | cagcaccaac agcagcacca acagcaccaa cagcagcagg aggtaatctc cgaaggcagc |
| 601 | agcgcaagag ccagggaggc cacgggggct ccctcttcct ccaaggatag ttacctaggg |
| 661 | ggcaattcaa ccatatctga cagtgccaag gagttgtgta aagcagtgtc tgtgtccatg |
| 721 | ggattgggtg tggaagcatt ggaacatctg agtccagggg aacagcttcg gggagactgc |
| 781 | atgtacgcgt cgctcctggg aggtccaccc gcggtgcgtc ccactccttg tgcgccgctg |
| 841 | cccgaatgca aaggtcttcc cctggacgaa ggcccaggca aaagcactga agagactgct |
| 901 | gagtattcct ctttcaaggg aggttacgcc aaaggattgg aaggtgagag cttggggtgc |
| 961 | tctggcagca gtgaagcagg tagctctggg acacttgaga tcccgtcctc tctgtctctg |
| 1021 | tataaatctg gagcactaga cgaggcagca gcataccaga atcgcgacta ctacaacttt |
| 1081 | ccgctggctc tgtccgggcc gccgcacccc ccgcccccta cccatccaca cgcccgtatc |
| 1141 | aagctggaga acccattgga ctacggcagc gcctgggctg cggcggcagc gcaatgccgc |
| 1201 | tatggggact tgggtagtct acatggaggg agtgtagccg ggcccagcac tggatcgccc |
| 1261 | ccagccacca cctcttcttc ctggcatact ctcttcacag ctgaagaagg ccaattatat |
| 1321 | gggccaggag gcgggggcgg cagcagcagc ccaagcgatg ccgggcctgt agccccctat |
| 1381 | ggctacactc ggccccctca ggggctgaca agccaggaga gtgactactc tgcctccgaa |
| 1441 | gtgtggtacc ctggtggagt tgtgaacaga gtaccctatc ccagtcccaa ttgtgtcaaa |
| 1501 | agtgaaatgg gaccttggat ggagaactac tccggacctt atggggacat gcgtttggac |
| 1561 | agtaccaggg accatgtttt acccatcgac tattactttc caccccagaa gacctgcctg |
| 1621 | atctgtggag atgaagcttc tggctgtcac tacggagctc tcacttgtgg cagctgcaag |
| 1681 | gtcttcttca aaagagccgc tgaagggaaa cagaagtatc tatgtgccag cagaaacgat |
| 1741 | tgtaccattg ataaatttcg gaggaaaaat tgcccatctt gtcgtctccg gaaatgttat |
| 1801 | gaagcaggga tgactctggg agctcgtaag ctgaagaaac ttggaaatct aaaactacag |
| 1861 | gaggaaggag aaaactccaa tgctggcagc cccactgagg acccatccca gaagatgact |
| 1921 | gtatcacaca ttgaaggcta tgaatgtcag cctatctttc ttaacgtcct ggaagccatt |
| 1981 | gagccaggag tggtgtgtgc cggacatgac aacaaccaac cagattcctt tgctgccttg |
| 2041 | ttatctagcc tcaatgagct tggagagagg cagcttgtgc atgtggtcaa gtgggccaag |
| 2101 | gccttgcctg gcttccgcaa cttgcatgtg gatgaccaga tggcggtcat tcagtattcc |
| 2161 | tggatgggac tgatggtatt tgccatgggt tggcggtcct tcactaatgt caactccagg |
| 2221 | atgctctact ttgcacctga cttggttttc aatgagtacc gcatgcacaa gtctcggatg |
| 2281 | tacagccagt gtgtgaggat gaggcacctg tctcaagagt ttggatggct ccaaataacc |
| 2341 | ccccaggaat tcctgtgcat gaaagcactg ctgctcttca gcattattcc agtggatggg |
| 2401 | ctgaaaaatc aaaaattctt tgatgaactt cgaatgaact acatcaagga actcgatcgc |
| 2461 | atcattgcat gcaaaagaaa gaatcccaca tcctgctcaa ggcgcttcta ccagctcacc |
| 2521 | aagctcctgg attctgtgca gcctattgca agagagctgc atcagttcac ttttgacctg |
| 2581 | ctaatcaagt cccatatggt gagcgtggac tttcctgaaa tgatggcaga gatcatctct |
| 2641 | gtgcaagtgc ccaagatcct ttctgggaaa gtcaagccca tctatttcca cacacagtga |
| 30. SEQ ID NO:30 Genbank Accession No. NM_000044 Human AR mRNA | |
| 1 | cgagatcccg gggagccagc ttgctgggag agcgggacgg tccggagcaa gcccacaggc |
| 61 | agaggaggcg acagagggaa aaagggccga gctagccgct ccagtgctgt acaggagccg |
| 121 | aagggacgca ccacgccagc cccagcccgg ctccagcgac agccaacgcc tcttgcagcg |
| 181 | cggcggcttc gaagccgccg cccggagctg ccctttcctc ttcggtgaag tttttaaaag |
| 241 | ctgctaaaga ctcggaggaa gcaaggaaag tgcctggtag gactgacggc tgcctttgtc |
| 301 | ctcctcctct ccaccccgcc tccccccacc ctgccttccc cccctccccc gtcttctctc |
| 361 | ccgcagctgc ctcagtcggc tactctcagc caacccccct caccaccctt ctccccaccc |
| 421 | gcccccccgc ccccgtcggc ccagcgctgc cagcccgagt ttgcagagag gtaactccct |
| 481 | ttggctgcga gcgggcgagc tagctgcaca ttgcaaagaa ggctcttagg agccaggcga |
| 541 | ctggggagcg gcttcagcac tgcagccacg acccgcctgg ttagaattcc ggcggagaga |
| 601 | accctctgtt ttcccccact ctctctccac ctcctcctgc cttccccacc ccgagtgcgg |
| 661 | agcagagatc aaaagatgaa aaggcagtca ggtcttcagt agccaaaaaa caaaacaaac |
| 721 | aaaaacaaaa aagccgaaat aaaagaaaaa gataataact cagttcttat ttgcacctac |
| 781 | ttcagtggac actgaatttg gaaggtggag gattttgttt ttttctttta agatctgggc |
| 841 | atcttttgaa tctacccttc aagtattaag agacagactg tgagcctagc agggcagatc |
| 901 | ttgtccaccg tgtgtcttct tctgcacgag actttgaggc tgtcagagcg ctttttgcgt |
| 961 | ggttgctccc gcaagtttcc ttctctggag cttcccgcag gtgggcagct agctgcagcg |
| 1021 | actaccgcat catcacagcc tgttgaactc ttctgagcaa gagaagggga ggcggggtaa |
| 1081 | gggaagtagg tggaagattc agccaagctc aaggatggaa gtgcagttag ggctgggaag |
| 1141 | ggtctaccct cggccgccgt ccaagaccta ccgaggagct ttccagaatc tgttccagag |
| 1201 | cgtgcgcgaa gtgatccaga acccgggccc caggcaccca gaggccgcga gcgcagcacc |
| 1261 | tcccggcgcc agtttgctgc tgctgcagca gcagcagcag cagcagcagc agcagcagca |
| 1321 | gcagcagcag cagcagcagc agcagcaaga gactagcccc aggcagcagc agcagcagca |
| 1381 | gggtgaggat ggttctcccc aagcccatcg tagaggcccc acaggctacc tggtcctgga |
| 1441 | tgaggaacag caaccttcac agccgcagtc ggccctggag tgccaccccg agagaggttg |
| 1501 | cgtcccagag cctggagccg ccgtggccgc cagcaagggg ctgccgcagc agctgccagc |
| 1561 | acctccggac gaggatgact cagctgcccc atccacgttg tccctgctgg gccccacttt |
| 1621 | ccccggctta agcagctgct ccgctgacct taaagacatc ctgagcgagg ccagcaccat |
| 1681 | gcaactcctt cagcaacagc agcaggaagc agtatccgaa ggcagcagca gcgggagagc |
| 1741 | gagggaggcc tcgggggctc ccacttcctc caaggacaat tacttagggg gcacttcgac |
| 1801 | catttctgac aacgccaagg agttgtgtaa ggcagtgtcg gtgtccatgg gcctgggtgt |
| 1861 | ggaggcgttg gagcatctga gtccagggga acagcttcgg ggggattgca tgtacgcccc |
| 1921 | acttttggga gttccacccg ctgtgcgtcc cactccttgt gccccattgg ccgaatgcaa |
| 1981 | aggttctctg ctagacgaca gcgcaggcaa gagcactgaa gatactgctg agtattcccc |
| 2041 | tttcaaggga ggttacacca aagggctaga aggcgagagc ctaggctgct ctggcagcgc |
| 2101 | tgcagcaggg agctccggga cacttgaact gccgtctacc ctgtctctct acaagtccgg |
| 2161 | agcactggac gaggcagctg cgtaccagag tcgcgactac tacaactttc cactggctct |
| 2221 | ggccggaccg ccgccccctc cgccgcctcc ccatccccac gctcgcatca agctggagaa |
| 2281 | cccgctggac tacggcagcg cctgggcggc tgcggcggcg cagtgccgct atggggacct |
| 2341 | ggcgagcctg catggcgcgg gtgcagcggg acccggttct gggtcaccct cagccgccgc |
| 2401 | ttcctcatcc tggcacactc tcttcacagc cgaagaaggc cagttgtatg gaccgtgtgg |
| 2461 | tggtggtggg ggtggtggcg gcggcggcgg cggcggcggc ggcggcggcg gcggcggcgg |
| 2521 | cggcggcggc gaggcgggag ctgtagcccc ctacggctac actcggcccc ctcaggggct |
| 2581 | ggcgggccag gaaagcgact tcaccgcacc tgatgtgtgg taccctggcg gcatggtgag |
| 2641 | cagagtgccc tatcccagtc ccacttgtgt caaaagcgaa atgggcccct ggatggatag |
| 2701 | ctactccgga ccttacgggg acatgcgttt ggagactgcc agggaccatg ttttgcccat |
| 2761 | tgactattac tttccacccc agaagacctg cctgatctgt ggagatgaag cttctgggtg |
| 2821 | tcactatgga gctctcacat gtggaagctg caaggtcttc ttcaaaagag ccgctgaagg |
| 2881 | gaaacagaag tacctgtgcg ccagcagaaa tgattgcact attgataaat tccgaaggaa |
| 2941 | aaattgtcca tcttgtcgtc ttcggaaatg ttatgaagca gggatgactc tgggagcccg |
| 3001 | gaagctgaag aaacttggta atctgaaact acaggaggaa ggagaggctt ccagcaccac |
| 3061 | cagccccact gaggagacaa cccagaagct gacagtgtca cacattgaag gctatgaatg |
| 3121 | tcagcccatc tttctgaatg tcctggaagc cattgagcca ggtgtagtgt gtgctggaca |
| 3181 | cgacaacaac cagcccgact cctttgcagc cttgctctct agcctcaatg aactgggaga |
| 3241 | gagacagctt gtacacgtgg tcaagtgggc caaggccttg cctggcttcc gcaacttaca |
| 3301 | cgtggacgac cagatggctg tcattcagta ctcctggatg gggctcatgg tgtttgccat |
| 3361 | gggctggcga tccttcacca atgtcaactc caggatgctc tacttcgccc ctgatctggt |
| 3421 | tttcaatgag taccgcatgc acaagtcccg gatgtacagc cagtgtgtcc gaatgaggca |
| 3481 | cctctctcaa gagtttggat ggctccaaat caccccccag gaattcctgt gcatgaaagc |
| 3541 | actgctactc ttcagcatta ttccagtgga tgggctgaaa aatcaaaaat tctttgatga |
| 3601 | acttcgaatg aactacatca aggaactcga tcgtatcatt gcatgcaaaa gaaaaaatcc |
| 3661 | cacatcctgc tcaagacgct tctaccagct caccaagctc ctggactccg tgcagcctat |
| 3721 | tgcgagagag ctgcatcagt tcacttttga cctgctaatc aagtcacaca tggtgagcgt |
| 3781 | ggactttccg gaaatgatgg cagagatcat ctctgtgcaa gtgcccaaga tcctttctgg |
| 3841 | gaaagtcaag cccatctatt tccacaccca gtgaagcatt ggaaacccta tttccccacc |
| 3901 | ccagctcatg ccccctttca gatgtcttct gcctgttata actctgcact actcctctgc |
| 3961 | agtgccttgg ggaatttcct ctattgatgt acagtctgtc atgaacatgt tcctgaattc |
| 4021 | tatttgctgg gctttttttt tctctttctc tcctttcttt ttcttcttcc ctccctatct |
| 4081 | aaccctccca tggcaccttc agactttgct tcccattgtg gctcctatct gtgttttgaa |
| 4141 | tggtgttgta tgcctttaaa tctgtgatga tcctcatatg gcccagtgtc aagttgtgct |
| 4201 | tgtttacagc actactctgt gccagccaca caaacgttta cttatcttat gccacgggaa |
| 4261 | gtttagagag ctaagattat ctggggaaat caaaacaaaa aacaagcaaa caaaaaaaaa |
| 4321 | a |
| 31. SEQ ID NO:31 Genbank Accession No. NM_000044 Human AR protein | |
| sequence | |
| MEVQLGLGRVYPRPPSKTYRGAFQNLFQSVREVIQNPGPRHPEA | |
| ASAAPPGASLLLLQQQQQQQQQQQQQQQQQQQQQETSPRQQQQQQGEDGSPQAHRRGP | |
| TGYLVLDEEQQPSQPQSALECHPERGCVPEPGAAVAASKGLPQQLPAPPDEDDSAAPS | |
| TLSLLGPTFPGLSSCSADLKDILSEASTMQLLQQQQQEAVSEGSSSGRAREASGAPTS | |
| SKDNYLGGTSTISDNAKELCKAVSVSMGLGVEALEHLSPGEQLRGDCMYAPLLGVPPA | |
| VRPTPCAPLAECKGSLLDDSAGKSTEDTAEYSPFKGGYTKGLEGESLGCSGSAAAGSS | |
| GTLELPSTLSLYKSGALDEAAAYQSRDYYNFPLALAGPPPPPPPPHPHARIKLENPLD | |
| YGSAWAAAAAQCRYGDLASLHGAGAAGPGSGSPSAAASSSWHTLFTAEEGQLYGPCGG | |
| GGGGGGGGGGGGGGGGGGGGGGEAGAVAPYGYTRPPQGLAGQESDFTAPDVWYPGGMV | |
| SRVPYPSPTCVKSEMGPWMDSYSGPYGDMRLETARDHVLPIDYYFPPQKTCLICGDEA | |
| SGCHYGALTCGSCKVFFKRAAEGKQKYLCASRNDCTIDKFRRKNCPSCRLRKCYEAGM | |
| TLGARKLKKLGNLKLQEEGEASSTTSPTEETTQKLTVSHIEGYECQPIFLNVLEAIEP | |
| GVVCAGHDNNQPDSFAALLSSLNELGERQLVHVVKWAKALPGFRNLHVDDQMAVIQYS | |
| WMGLMVFAMGWRSFTNVNSRMLYFAPDLVFNEYRMHKSRMYSQCVRMRHLSQEFGWLQ | |
| ITPQEFLCMKALLLFSIIPVDGLKNQKFFDELRMNYIKELDRIIACKRKNPTSCSRRF | |
| YQLTKLLDSVQPIARELHQFTFDLLIKSHMVSVDFPEMMAEIISVQVPKILSGKVKPI | |
| YFHTQ | |
| 32. SEQ ID NO:32 Genbank accession number X03635. for Human protein | |
| sequence of an estrogen receptor | |
| MTMTLHTKASGMALLHQIQGNELEPLNRPQLKIPLERPLGEVYLDSSKPAVYNYPEGAAYEFNAAA | |
| AANAQVYGQTGLPYGPGSEAAAFGSNGLGGFPPLNSVSPSPLMLLHPPPQLSPFLQPHGQQVPYYLE | |
| NEPSGYTVREAGPPAFYRPNSDNRRQGGRERLASTNDKGSMAMESAKETRYCAVCNDYASGYHY | |
| GVWSCEGCKAFFKRSIQGHNDYMCPATNQCTIDKNRRKSCQACRLRKCYEVGMMKGGIRKDRRG | |
| GRMLKHKRQRDDGEGRGEVGSAGDMRAANLWPSPLMIKRSKKNSLALSLTADQMVSALLDAEPPI | |
| LYSEYDPTRPFSEASMMGLLTNLADRELVHMINWAKRVPGFVDLTLHDQVHLLECAWLEILMIGLV | |
| WRSMEHPVKLLFAPNLLLDRNQGKCVEGMVEIFDMLLATSSRFRMMNLQGEEFVCLKSIILLNSGV | |
| YTFLSSTLKSLEEKDHIHRVLDKITDTLIHLMAKAGLTLQQQHQRLAQLLLILSHIRHMSNKGMEHL | |
| YSMKCKNVVPLYDLLLEMLDAHRLHAPTSRGGASVEETDQSHLATAGSTSSHSLQKYYITGEAEGF | |
| PATV | |
| 33. SEQ ID NO:33 Genbank accession number X03635. for Human mRNA | |
| sequence of an estrogen receptor | |
| 1 | gagttgtgcc tggagtgatg tttaagccaa tgtcagggca aggcaacagt ccctggccgt |
| 61 | cctccagcac ctttgtaatg catatgagct cgggagacca gtacttaaag ttggaggccc |
| 121 | gggagcccag gagctggcgg agggcgttcg tcctgggagc tgcacttgct ccgtcgggtc |
| 181 | gccggcttca ccggaccgca ggctcccggg gcagggccgg ggccagagct cgcgtgtcgg |
| 241 | cgggacatgc gctgcgtcgc ctctaacctc gggctgtgct ctttttccag gtggcccgcc |
| 301 | ggtttctgag ccttctgccc tgcggggaca cggtctgcac cctgcccgcg gccacggacc |
| 361 | atgaccatga ccctccacac caaagcatct gggatggccc tactgcatca gatccaaggg |
| 421 | aacgagctgg agcccctgaa ccgtccgcag ctcaagatcc ccctggagcg gcccctgggc |
| 481 | gaggtgtacc tggacagcag caagcccgcc gtgtacaact accccgaggg cgccgcctac |
| 541 | gagttcaacg ccgcggccgc cgccaacgcg caggtctacg gtcagaccgg cctcccctac |
| 601 | ggccccgggt ctgaggctgc ggcgttcggc tccaacggcc tggggggttt ccccccactc |
| 661 | aacagcgtgt ctccgagccc gctgatgcta ctgcacccgc cgccgcagct gtcgcctttc |
| 721 | ctgcagcccc acggccagca ggtgccctac tacctggaga acgagcccag cggctacacg |
| 781 | gtgcgcgagg ccggcccgcc ggcattctac aggccaaatt cagataatcg acgccagggt |
| 841 | ggcagagaaa gattggccag taccaatgac aagggaagta tggctatgga atctgccaag |
| 901 | gagactcgct actgtgcagt gtgcaatgac tatgcttcag gctaccatta tggagtctgg |
| 961 | tcctgtgagg gctgcaaggc cttcttcaag agaagtattc aaggacataa cgactatatg |
| 1021 | tgtccagcca ccaaccagtg caccattgat aaaaacagga ggaagagctg ccaggcctgc |
| 1081 | cggctccgca aatgctacga agtgggaatg atgaaaggtg ggatacgaaa agaccgaaga |
| 1141 | ggagggagaa tgttgaaaca caagcgccag agagatgatg gggagggcag gggtgaagtg |
| 1201 | gggtctgctg gagacatgag agctgccaac ctttggccaa gcccgctcat gatcaaacgc |
| 1261 | tctaagaaga acagcctggc cttgtccctg acggccgacc agatggtcag tgccttgttg |
| 1321 | gatgctgagc cccccatact ctattccgag tatgatccta ccagaccctt cagtgaagct |
| 1381 | tcgatgatgg gcttactgac caacctggca gacagggagc tggttcacat gatcaactgg |
| 1441 | gcgaagaggg tgccaggctt tgtggatttg accctccatg atcaggtcca ccttctagaa |
| 1501 | tgtgcctggc tagagatcct gatgattggt ctcgtctggc gctccatgga gcacccagtg |
| 1561 | aagctactgt ttgctcctaa cttgctcttg gacaggaacc agggaaaatg tgtagagggc |
| 1621 | atggtggaga tcttcgacat gctgctggct acatcatctc ggttccgcat gatgaatctg |
| 1681 | cagggagagg agtttgtgtg cctcaaatct attattttgc ttaattctgg agtgtacaca |
| 1741 | tttctgtcca gcaccctgaa gtctctggaa gagaaggacc atatccaccg agtcctggac |
| 1801 | aagatcacag acactttgat ccacctgatg gccaaggcag gcctgaccct gcagcagcag |
| 1861 | caccagcggc tggcccagct cctcctcatc ctctcccaca tcaggcacat gagtaacaaa |
| 1921 | ggcatggagc atctgtacag catgaagtgc aagaacgtgg tgcccctcta tgacctgctg |
| 1981 | ctggagatgc tggacgccca ccgcctacat gcgcccacta gccgtggagg ggcatccgtg |
| 2041 | gaggagacgg accaaagcca cttggccact gcgggctcta cttcatcgca ttccttgcaa |
| 2101 | aagtattaca tcacggggga ggcagagggt ttccctgcca cagtctgaga gctccctggc |
| 2161 | tcccacacgg ttcagataat ccctgctgca ttttaccctc atcatgcacc actttagcca |
| 2221 | aattctgtct cctgcataca ctccggcatg catccaacac caatggcttt ctagatgagt |
| 2281 | ggccattcat ttgcttgctc agttcttagt ggcacatctt ctgtcttctg ttgggaacag |
| 2341 | ccaaagggat tccaaggcta aatctttgta acagctctct ttcccccttg ctatgttact |
| 2401 | aagcgtgagg attcccgtag ctcttcacag ctgaactcag tctatgggtt ggggctcaga |
| 2461 | taactctgtg catttaagct acttgtagag acccaggcct ggagagtaga cattttgcct |
| 2521 | ctgataagca ctttttaaat ggctctaaga ataagccaca gcaaagaatt taaagtggct |
| 2581 | cctttaattg gtgacttgga gaaagctagg tcaagggttt attatagcac cctcttgtat |
| 2641 | tcctatggca atgcatcctt ttatgaaagt ggtacacctt aaagctttta tatgactgta |
| 2701 | gcagagtatc tggtgattgt caattcactt ccccctatag gaatacaagg ggccacacag |
| 2761 | ggaaggcaga tcccctagtt ggccaagact tattttaact tgatacactg cagattcaga |
| 2821 | gtgtcctgaa gctctgcctc tggctttccg gtcatgggtt ccagttaatt catgcctccc |
| 2881 | atggacctat ggagagcaac aagttgatct tagttaagtc tccctatatg agggataagt |
| 2941 | tcctgatttt tgtttttatt tttgtgttac aaaagaaagc cctccctccc tgaacttgca |
| 3001 | gtaaggtcag cttcaggacc tgttccagtg ggcactgtac ttggatcttc ccggcgtgtg |
| 3061 | tgtgccttac acaggggtga actgttcact gtggtgatgc atgatgaggg taaatggtag |
| 3121 | ttgaaaggag caggggccct ggtgttgcat ttagccctgg ggcatggagc tgaacagtac |
| 3181 | ttgtgcagga ttgttgtggc tactagagaa caagagggaa agtagggcag aaactggata |
| 3241 | cagttctgag cacagccaga cttgctcagg tggccctgca caggctgcag ctacctagga |
| 3301 | acattccttg cagaccccgc attgcctttg ggggtgccct gggatccctg gggtagtcca |
| 3361 | gctcttattc atttcccagc gtggccctgg ttggaagaag cagctgtcaa gttgtagaca |
| 3421 | gctgtgttcc tacaattggc ccagcaccct ggggcacggg agaagggtgg ggaccgttgc |
| 3481 | tgtcactact caggctgact ggggcctggt cagattacgt atgcccttgg tggtttagag |
| 3541 | ataatccaaa atcagggttt ggtttgggga agaaaatcct cccccttcct cccccgcccc |
| 3601 | gttccctacc gcctccactc ctgccagctc atttccttca atttcctttg acctataggc |
| 3661 | taaaaaagaa aggctcattc cagccacagg gcagccttcc ctgggccttt gcttctctag |
| 3721 | cacaattatg ggttacttcc tttttcttaa caaaaaagaa tgtttgattt cctctgggtg |
| 3781 | accttattgt ctgtaattga aaccctattg agaggtgatg tctgtgttag ccaatgaccc |
| 3841 | aggtagctgc tcgggcttct cttggtatgt cttgtttgga aaagtggatt tcattcattt |
| 3901 | ctgattgtcc agttaagtga tcaccaaagg actgagaatc tgggagggca aaaaaaaaaa |
| 3961 | aaaaagtttt tatgtgcact taaatttggg gacaatttta tgtatctgtg ttaaggatat |
| 4021 | gcttaagaac ataattcttt tgttgctgtt tgtttaagaa gcaccttagt ttgtttaaga |
| 4081 | agcaccttat atagtataat atatattttt ttgaaattac attgcttgtt tatcagacaa |
| 4141 | ttgaatgtag taattctgtt ctggatttaa tttgactggg ttaacatgca aaaaccaagg |
| 4201 | aaaaatattt agtttttttt tttttttttg tatacttttc aagctacctt gtcatgtata |
| 4261 | cagtcattta tgcctaaagc ctggtgatta ttcatttaaa tgaagatcac atttcatatc |
| 4321 | aacttttgta tccacagtag acaaaatagc actaatccag atgcctattg ttggatattg |
| 4381 | aatgacagac aatcttatgt agcaaagatt atgcctgaaa aggaaaatta ttcagggcag |
| 4441 | ctaattttgc ttttaccaaa atatcagtag taatattttt ggacagtagc taatgggtca |
| 4501 | gtgggttctt tttaatgttt atacttagat tttcttttaa aaaaattaaa ataaaacaaa |
| 4561 | aaaaatttct aggactagac gatgtaatac cagctaaagc caaacaatta tacagtggaa |
| 4621 | ggttttacat tattcatcca atgtgtttct attcatgtta agatactact acatttgaag |
| 4681 | tgggcagaga acatcagatg attgaaatgt tcgcccaggg gtctccagca actttggaaa |
| 4741 | tctctttgta tttttacttg aagtgccact aatggacagc agatattttc tggctgatgt |
| 4801 | tggtattggg tgtaggaaca tgatttaaaa aaaaaactct tgcctctgct ttcccccact |
| 4861 | ctgaggcaag ttaaaatgta aaagatgtga tttatctggg gggctcaggt atggtgggga |
| 4921 | agtggattca ggaatctggg gaatggcaaa tatattaaga agagtattga aagtatttgg |
| 4981 | aggaaaatgg ttaattctgg gtgtgcacca aggttcagta gagtccactt ctgccctgga |
| 5041 | gaccacaaat caactagctc catttacagc catttctaaa atggcagctt cagttctaga |
| 5101 | gaagaaagaa caacatcagc agtaaagtcc atggaatagc tagtggtctg tgtttctttt |
| 5161 | cgccattgcc tagcttgccg taatgattct ataatgccat catgcagcaa ttatgagagg |
| 5221 | ctaggtcatc caaagagaag accctatcaa tgtaggttgc aaaatctaac ccctaaggaa |
| 5281 | gtgcagtctt tgatttgatt tccctagtaa ccttgcagat atgtttaacc aagccatagc |
| 5341 | ccatgccttt tgagggctga acaaataagg gacttactga taatttactt ttgatcacat |
| 5401 | taaggtgttc tcaccttgaa atcttataca ctgaaatggc cattgattta ggccactggc |
| 5461 | ttagagtact ccttcccctg catgacactg attacaaata ctttcctatt catactttcc |
| 5521 | aattatgaga tggactgtgg gtactgggag tgatcactaa caccatagta atgtctaata |
| 5581 | ttcacaggca gatctgcttg gggaagctag ttatgtgaaa ggcaaataaa gtcatacagt |
| 5641 | agctcaaaag gcaaccataa ttctctttgg tgcaagtctt gggagcgtga tctagattac |
| 5701 | actgcaccat tcccaagtta atcccctgaa aacttactct caactggagc aaatgaactt |
| 5761 | tggtcccaaa tatccatctt ttcagtagcg ttaattatgc tctgtttcca actgcatttc |
| 5821 | ctttccaatt gaattaaagt gtggcctcgt ttttagtcat ttaaaattgt tttctaagta |
| 5881 | attgctgcct ctattatggc acttcaattt tgcactgtct tttgagattc aagaaaaatt |
| 5941 | tctattcatt tttttgcatc caattgtgcc tgaactttta aaatatgtaa atgctgccat |
| 6001 | gttccaaacc catcgtcagt gtgtgtgttt agagctgtgc accctagaaa caacatactt |
| 6061 | gtcccatgag caggtgcctg agacacagac ccctttgcat tcacagagag gtcattggtt |
| 6121 | atagagactt gaattaataa gtgacattat gccagtttct gttctctcac aggtgataaa |
| 6181 | caatgctttt tgtgcactac atactcttca gtgtagagct cttgttttat gggaaaaggc |
| 6241 | tcaaatgcca aattgtgttt gatggattaa tatgcccttt tgccgatgca tactattact |
| 6301 | gatgtgactc ggttttgtcg cagctttgct ttgtttaatg aaacacactt gtaaacctct |
| 6361 | tttgcacttt gaaaaagaat ccagcgggat gctcgagcac ctgtaaacaa ttttctcaac |
| 6421 | ctatttgatg ttcaaataaa gaattaaact |

What is claimed is:

1. A composition comprising a fragment of TR2, wherein the composition interacts with ER, such that ER transcriptional activity is decreased relative to transcriptional activity in the absence of the composition.

2. A composition comprising a fragment of TR2, wherein the composition interacts with ER, such that ER transcriptional activity is decreased relative to transcriptional activity in the absence of the composition, wherein the fragment of TR2 has at least 80%, 85%, 90%, or 95% identity to amino acids 88-196 of SEQ ID NO:10.

3. The composition of claim 2, wherein any variation between the TR2 and the sequence set forth in SEQ ID NO: 10 is a considered a conserved variation.

4. The composition of claim 2, wherein the composition reduces the transcription activity of ER.

5. The composition of claims 2, wherein the composition reduces the transcription activity of ER by 10%, 25%, 50%, or 90%.

6. The composition of claim 2, wherein the composition reduces the G1/S transition of the cell cycle.

7. The composition of claims 2, wherein the composition reduces the G1/S transition of the cell cycle by 10%, 25%, 50%, or 90%.

8. A method of inhibiting transcription activity of ER comprising administering the composition of claim 1.

9. The method of claim 8, wherein the composition reduces the transcription activity of ER.

10. The method of claim 8, wherein the composition reduces the transcription activity of ER by 10%, 25%,50%, or 90%.

11. The method of claim 8, wherein the composition reduces the GI/S transition of the cell cycle.

12. The method of claim 8, wherein the composition reduces the G1/S transition of the cell cycle by 10%, 25%, 50%, or 90%.

13. A method of inhibiting TR2 transcription activity comprising administering a composition that binds TR2, wherein the composition is ER, ER fragment, ER variant, a molecule that competitively competes with TR2 for ER binding, or combination thereof.

14. A method of identifying an inhibitor of an interaction between ER and TR2, comprising incubating a library of molecules with an ER, ER fragment, ER variant or combination, forming a mixture, and identifying the molecules that disrupt the interaction between the ER, ER fragment, ER variant, or combination and TR2, wherein the interaction disrupted comprises an interaction between the ER, ER fragment, ER variant, or combination and TR2 binding site.

15. The method of claim 14, wherein the step of isolating comprises incubating the mixture with a molecule comprising TR2, TR2 fragment, TR2 variant, or combination.

16. A method of identifying an inhibitor of an interaction between ER and TR2 comprising incubating a library of molecules with TR2, TR2 fragment, TR2 variant or combination forming a mixture, and identifying the molecules that disrupt the interaction between ER and the TR2, TR2 fragment, TR2 variant or combination, wherein the interaction disrupted comprises an interaction between the ER and the TR, TR2 fragment, TR2 variant or combination binding site.

17. The method of claim 16, wherein the step of isolating comprises incubating the mixture with molecule comprising ER, ER fragment, ER variant, or combination.

18. A composition comprising a fragment of ER, wherein the composition interacts with TR2, such that TR2 transcriptional activity is decreased relative to transcriptional activity in the absence of the composition, wherein the fragment comprises a polypeptide having at least 80%, 85%, 90%, or 95% identity to the sequence set forth in SEQ ID NO:32, the fragment comprises a polypeptide having at least 80%, 85%, 90%, or 95% identity to amino acids 312-340, set forth in SEQ ID NO:32, the fragment comprises a polypeptide having at least 80%, 85%, 90%, or 95% identity to amino acids 123-340, set forth in SEQ ID NO:32, or the fragment comprises a polypeptide having at least 80%, 85%, 90%, or 95% identity to amino acids 312-595, set forth in SEQ ID NO:32.

19. The composition of claim 18, wherein any variation between the ER and the sequence set forth in SEQ ID NO: 32 is a conserved variation.

20. The composition of claim 18, wherein the fragment comprises the sequence set forth in SEQ ID NO:32, or is a conserved variant thereof.

21. The composition of claims 18, wherein the composition reduces the transcription activity of TR4.

22. The composition of claims 18, wherein the composition reduces the transcription activity of TR4 by 10%, 25%, 50%, or 90%.

23. A method of inhibiting transcription activity of TR2 comprising administering the composition of claims 18.

24. The composition of claims 23, wherein the composition reduces the transcription activity of TR4.

25. The composition of claims 23, wherein the composition reduces the transcription activity of TR4 by 10%, 25%, 50%, or 90%.

26. A method of identifying inhibitors of ER transcription activity comprising mixing a compound with ER, ER fragment, ER variant, or combination and identifying compounds which compete with TR2 binding with ER, ER fragment, ER variant, or combination.

27. A method of identifying inhibitors of ER transcription activity comprising mixing a set of compounds with TR2, TR2 fragment, TR variant, or combination and identifying compounds which compete with ER binding with TR2, TR2 fragment, TR variant, or combination.

28. The method of claims 15, wherein the TR comprises amino acids 88-196 of SEQ ID NO:10.

29. The method of claims 28, wherein the TR2 has a sequence of at least 80%, 85%, 90%, or 95% identity to amino acids 88-196 of SEQ ID NO:10.

30. The method of claims 29, wherein any variation between the TR2 and the sequence set forth in SEQ ID NO: 10 is considered a conserved variation.

31. The method of claims 14, wherein the identified compound binds ER with a k_{d} less than or equal to 10⁻⁵ M, 10⁻⁶ M, 10⁻⁷ M, 10⁻⁸ M, 10⁻⁹ M, or 10⁻¹⁰ M, 10⁻¹¹ M, or 10⁻¹² M.

32. The method of claims 14, wherein the ER comprises a polypeptide having at least 80%, 85%, 90%, or 95% identity to the sequence set forth in SEQ ID NO:32.

33. The method of claims 14, wherein the fragment comprises a polypeptide having at least 80%, 85%, 90%, or 95% identity to amino acids 312-340, set forth in SEQ ID NO:32, the fragment comprises a polypeptide having at least 80%, 85%, 90%, or 95% identity to amino acids 123-340, set forth in SEQ ID NO:32, or the fragment comprises a polypeptide having at least 80%, 85%, 90%, or 95% identity to amino acids 312-595, set forth in SEQ ID NO:32.

34. The method of claim 33, wherein any variation between the ER and the sequence set forth in SEQ ID NO: 32 is a conserved variation.

35. The method of claim 33, wherein the ER, ER fragment, ER variant or combination comprises the sequence set forth in SEQ ID NO:32, the sequence set forth by amino acids 312-340 of the sequence set forth in SEQ ID NO:32, the sequence set forth by amino acids 123-340 of the sequence set forth in SEQ ID NO:32, the sequence set forth by amino acids 312-595 of the sequence set forth in SEQ ID NO:32, or is a conserved variant thereof, or is a fragment thereof.

36. A method of inhibiting TR4 transcription activity comprising administering a composition that binds TR4, wherein the composition is AR, AR fragment, AR variant, a molecule that competitively competes with TR4 for AR binding, or a combination thereof.

37. A method of inhibiting AR transcription activity comprising administering a composition that binds AR, wherein the composition is TR4, TR4 fragment, TR4 variant, a molecule that competitively competes with AR for TR4 binding, or combination thereof.

38. A method of identifying an inhibitor of an interaction between AR and TR4, comprising incubating a library of molecules with AR, AR fragment, or AR variant, or combination forming a mixture, and identifying the molecules that disrupt the interaction between AR, AR fragment, or AR variant, or combination and TR4, wherein the interaction disrupted comprises an interaction between the AR, AR fragment, or AR variant, or combination and TR4 binding site.

39. The method of claim 38, wherein the step of isolating comprises incubating the mixture with molecule comprising TR4, TR4 fragment, TR4 variant or combination.

40. A method of identifying an inhibitor of an interaction between AR and TR4 comprising incubating a library of molecules with TR4, TR4 fragment, or TR4 variant, or combination forming a mixture, and identifying the molecules that disrupt the interaction between AR and the TR4, TR4 fragment, or TR4 variant, or combination, wherein the interaction disrupted comprises an interaction between the AR and the TR4, TR4 fragment, or TR4 variant, or combination binding site.

41. The method of claim 42, wherein the step of isolating comprises incubating the mixture with molecule comprising AR, AR fragment, AR variant or combination.

42. The method of claim 41, wherein the AR, AR fragment, AR variant or combination comprises a polypeptide having at least 80%, 85%, 90%, or 95% identity to SEQ ID NO:31.

43. The method of 42, wherein any variation between the AR and the sequence set forth in SEQ ID NO: 31 is a conserved variation.

44. The method of claim 36, wherein the transcription activity of TR4 is reduced by 10%, 25%, 50%, or 90%.

45. The method of claim 40, wherein the TR4, TR4 fragment, TR4 variant or combination comprises a polypeptide having at least 80%, 85%, 90%, or 95% identity to SEQ ID NO:16.

46. The method of 45, wherein any variation between the TR4 and the sequence set forth in SEQ ID NO: 16 is a conserved variation.

47. The method of claim 37, wherein the transcription activity of AR is reduced by 10%, 25%, 50%, or 90%.

48. A method of inhibiting TR4 transcription activity comprising administering a composition that binds TR4, wherein the composition is ER, ER fragment, ER variant, or a molecule that competitively competes with TR4 for ER binding or combination thereof.

49. A method of inhibiting ER transcription activity comprising administering a composition that binds ER, wherein the composition is TR4, TR4 fragment, TR4 variant, a molecule that competitively competes with ER for TR4 binding, or combination thereof.

50. A method of identifying an inhibitor of an interaction between ER and TR4, comprising incubating a library of molecules with ER, ER fragment, ER variant, or combination forming a mixture, and identifying the molecules that disrupt the interaction between the ER, ER fragment, ER variant, or combination and TR4, wherein the interaction disrupted comprises an interaction between the ER, ER fragment, ER variant, or combination and TR4 binding site.

51. The method of claim 50, wherein the step of isolating comprises incubating the mixture with molecule comprising TR4, TR4 fragment, TR4 variant or combination.

52. A method of identifying an inhibitor of an interaction between ER and TR4 comprising incubating a library of molecules with TR4, TR4 fragment, TR4 variant, or combination forming a mixture, and identifying the molecules that disrupt the interaction between ER and TR4, TR4 fragment, TR4 variant, or combination, wherein the interaction disrupted comprises an interaction between the ER and TR4, TR4 fragment, TR4 variant, or combination binding site.

53. The method of claim 51, wherein the step of isolating comprises incubating the mixture with a molecule comprising an ER, ER fragment, ER variant or combination.

54. The method of claim 53, wherein the ER, ER fragment, ER variant or combination comprises a polypeptide having at least 80%, 85%, 90%, or 95% identity to SEQ ID NO:32.

55. The method of 54, wherein any variation between the ER and the sequence set forth in SEQ ID NO: 32 is a conserved variation.

56. The method of claim 48, wherein the transcription activity of TR4 is reduced by 10%, 25%, 50%, or 90%.

57. The method of claim 52, wherein the TR4, TR4 fragment, TR4 variant or combination comprises a polypeptide having at least 80%, 85%, 90%, or 95% identity to SEQ ID NO:16.

58. The method of 57, wherein any variation between the TR4 and the sequence set forth in SEQ ID NO: 16 is a conserved variation.

59. The method of claim 49, wherein the transcription activity of ER is reduced by 10%, 25%, 50%, or 90%.

60. A method of inhibiting AR transcription activity comprising administering a composition that binds AR, wherein the composition is TR2, TR2 fragment, TR2 variant, a molecule that competitively competes with AR for TR2 binding or combination thereof.

61. A method of identifying an inhibitor of AR transcription activity comprising mixing a compound with AR, AR fragment, AR variant, or combination and identifying compounds which compete with the TR2 interaction with AR, AR fragment, AR variant, or combination.

62. A method of identifying inhibitors of AR transcription activity comprising mixing a set of compounds with TR2, TR2 fragment, TR2 variant or combination thereof and identifying compounds which compete with TR2, TR2 fragment, TR2 variant or combination interaction with AR.

63. A method of identifying an inhibitor of an interaction between AR and TR2, comprising incubating a library of molecules with AR, AR fragment, AR variant, or combination forming a mixture, and identifying the molecules that disrupt the interaction between AR, AR fragment, AR variant, or combination and TR2, wherein the interaction disrupted comprises an interaction between the AR, AR fragment, AR variant, or combination and TR2 binding site.

64. The method of claim 62, wherein the step of isolating comprises incubating the mixture with molecule comprising TR2, TR2 fragment, TR2 variant or combination.

65. A method of identifying an inhibitor of an interaction between AR and TR2 comprising incubating a library of molecules with TR2, TR2 fragment, TR2 variant, or combination forming a mixture, and identifying the molecules that disrupt the interaction between AR and the TR2, TR2 fragment, TR2 variant, or combination, wherein the interaction disrupted comprises an interaction between the AR and the TR2, TR2 fragment, TR2 variant, or combination binding site.

66. The method of claim 65, wherein the step of isolating comprises incubating the mixture with molecule comprising AR, AR fragment, AR variant or combination.

67. The method of claim 63, wherein the AR, AR fragment, AR variant or combination comprises a polypeptide having at least 80%, 85%, 90%, or 95% identity to SEQ ID NO:31.

68. The method of 67, wherein any variation between the AR and the sequence set forth in SEQ ID NO:31 is a conserved variation.

69. The method of claim 68, wherein the transcription activity of TR2 is reduced by 10%, 25%, 50%, or 90%.

70. The method of claim 65, wherein the TR2, TR2 fragment, TR2 variant or combination comprises a polypeptide having at least 80%, 85%, 90%, or 95% identity to SEQ ID NO:16.

71. The method of 70, wherein any variation between the TR2 and the sequence set forth in SEQ ID NO:16 is a conserved variation.

72. The method of claim 60, wherein the transcription activity of AR is reduced by 10%, 25%, 50%, or 90%.

73. An isolated composition comprising TR2 and ER or variants or fragments of either, TR2 and AR or variants or fragments of either, TR4 and ER or variants or fragments of either, or TR4 and AR or variants or fragments of either.


SEQUENCE LISTING (8 sequences)

SEQ ID NO: 1 - DNA, 12 bp, Artificial Sequence
  Description of Artificial Sequence/note = synthetic construct
aggtcaaggtca

SEQ ID NO: 2 - DNA, 13 bp, Artificial Sequence
  Description of Artificial Sequence/note = synthetic construct
aggtcanaggtca

SEQ ID NO: 3 - DNA, 14 bp, Artificial Sequence
  Description of Artificial Sequence/note = synthetic construct
aggtcannaggtca

SEQ ID NO: 4 - DNA, 15 bp, Artificial Sequence
  Description of Artificial Sequence/note = synthetic construct
aggtcannnaggtca

SEQ ID NO: 5 - DNA, 16 bp, Artificial Sequence
  Description of Artificial Sequence/note = synthetic construct
aggtcannnnaggtca

SEQ ID NO: 6 - DNA, 17 bp, Artificial Sequence
  Description of Artificial Sequence/note = synthetic construct
aggtcannnnnaggtca

SEQ ID NO: 7 - DNA, 18 bp, Artificial Sequence
  Description of Artificial Sequence/note = synthetic construct
aggtcannnnnnaggtca

SEQ ID NO: 8 - DNA, 15 bp, Artificial Sequence
  Description of Artificial Sequence/note = synthetic construct
ggwacannntgttct